(19) United States
(12) Patent Application Publication
(10) Pub. No.: US 2023/0193392 A1
(43) Pub. Date: Jun. 22, 2023

(54) Methods Of Identifying And Evaluating Liver Inflammation And Liver Fibrosis In A Subject By Determining A Stratified Score Based On Gene Expression

(71) Applicant: Regeneron Pharmaceuticals, Inc., Tarrytown, NY (US)

(72) Inventors: Allen Lin, Tarrytown, NY (US); Gabor Halasz, Tarrytown, NY (US); Xiping Cheng, Tarrytown, NY (US); Matthew Wipperman, Tarrytown, NY (US); Satyajit Karnik, Tarrytown, NY (US); Michael Edward Burczynski, Tarrytown, NY (US)

(21) Appl. No.: 18/067,982

(22) Filed: Dec. 19, 2022

Related U.S. Application Data
(60) Provisional application No. 63/291,575, filed on Dec. 20, 2021.
(60) Provisional application No. 63/292,000, filed on Dec. 21, 2021.

(51) Int. Cl. C12Q 1/6883

(52) U.S. Cl. CPC C12Q 1/6883; C12Q 2600/158; C12Q 1/6869

(57) ABSTRACT

The present disclosure provides methods of identifying and/or evaluating liver inflammation and liver fibrosis in a subject, and methods of tracking progression or remission of liver inflammation and/or liver fibrosis in a subject.

### FIELD

The present disclosure relates generally to methods of identifying and/or evaluating liver inflammation and liver fibrosis in a subject, and methods of treating subjects having liver inflammation or liver fibrosis comprising determining or having determined a subject's Transcriptome Score (TS) and administering an agent that treats or inhibits liver inflammation and/or liver fibrosis.

### BACKGROUND

Nonalcoholic steatohepatitis (NASH) is an advanced form of non-alcoholic fatty liver disease (NAFLD). NAFLD is caused by buildup of fat in the liver. When this buildup causes inflammation and damage, it is known as NASH. The current standard for assessing the presence and severity of NASH is histopathological assessment of core needle liver biopsy. The development of NASH is currently assessed by determining an NAFLD activity score (NAS) for steatosis, inflammation, and ballooning as well as an F score, an Ishak score, or a METAVIR score for fibrosis. For example, the NAS inflammation score is typically scored as: no foci, 0; <2 foci/200×, 1; 2-4 foci/200×, 2; and >4 foci/200×, 3, while fibrosis is typically scored as: no fibrosis, 0; portal fibrosis without septa, 1; portal fibrosis with septa, 2; bridging fibrosis, 3; cirrhosis, 4. However, these scores rely on subjective interpretation by histopathologists and can yield discordant results. Furthermore, the current scoring criteria generate highly granular, discrete data that lack nuance. Accordingly, there is a long felt but unmet need for an assessment of liver inflammation and fibrosis that is more continuous in terms of output and that lacks the discordant results that subjective interpretations can yield.

### SUMMARY

The present disclosure provides methods of identifying and/or evaluating liver inflammation or liver fibrosis in a subject, the method comprising: determining or having determined a subject's Transcriptome Score (TS), wherein the TS comprises a value determined from RNA expression of genes in a liver sample from the subject, and when the subject's TS is greater than a threshold TS determined from a reference population of subjects without liver inflammation and/or without liver fibrosis, administering an agent that treats or inhibits liver inflammation and/or liver fibrosis and/or conducting a surgery on the subject.

The present disclosure provides methods of identifying and/or evaluating liver inflammation or liver fibrosis in a subject, the method comprising: determining or having determined a subject's TS, wherein the TS comprises a value determined from changes in RNA expression of genes of longitudinal liver samples from the subject, and administering an agent that treats or inhibits liver inflammation and/or liver fibrosis and/or conducting a surgery on the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying figures, which are incorporated in and constitute a part of this specification, illustrate several features of the present disclosure.

FIG. 1 (Panel A) shows a flow diagram of a representative gene panel selection, using n cohorts of patients exhibiting a trait of interest. Genes with median disease-stage transcript per million (TPM)s greater than a threshold TPM in at least m of these n cohorts are selected. For each of the cohorts, magnitudes of fold change and classification metrics (such as area under the curve or significance of fold change) of each gene for the desired disease comparison are computed. This results in 2n lists. Genes in each list are ranked by their value (e.g., descending order for magnitude of fold change, descending for area under the curve, ascending for p-value significance). The top-x genes from each list are selected. Genes that (1) appear in at least y out of the total 2n top-x lists and (2) are significant for fold change in at least z cohorts are selected for the gene panel. Genes in the gene panel are first ranked by the number of top-x lists they appear in (out of 2n) and then by their median rank across the 2n lists.

FIG. 1 (Panel B) shows a flow diagram of a representative gene panel evaluation. Within each cohort, each subject's TS for the enrichment of genes in the gene panel compared to those not in the gene panel is computed. Classification metrics are then computed reflecting the ability of the TSs to predict disease class. Subsets of the gene panel are also evaluated to understand the contribution of individual genes in the gene panel towards disease class classification.

FIG. 2 (Panel A) shows a representative distribution of the number of times a given gene appeared across the six top-200 gene fibrosis ranked lists, the expected distribution of the number of times a gene appears across six randomly selected lists of 200 genes, and the distribution of the number of times each gene in the selected fibrosis gene panel appeared across the six top-200 gene fibrosis ranked lists. The six fibrosis ranked lists are one list of fold change and one of precision-recall area under the curve, comparing fibrosis stage F3 and higher versus fibrosis stage F0 and F1, from each of the three GHS, REGN, and Govaere cohorts. Genes that appeared in at least three out of the six top-200 gene fibrosis ranked lists, as indicated by the horizontal black threshold line, and were statistically significant for fold change in at least two of the cohorts were selected to be in the fibrosis gene panel.

FIG. 2 (Panel B) shows a representative distribution of the number of times a given gene appeared across the four top-200 gene inflammation ranked lists, the expected distribution of the number of times a gene appears across four randomly selected lists of 200 genes, and the distribution of the number of times each gene in the selected inflammation gene panel appeared across the four top-200 gene inflammation ranked lists. The four inflammation ranked lists are one list of fold change and one of precision-recall area under the curve, comparing inflammation stage 2 and higher versus inflammation stage 0, from each of the two GHS and REGN cohorts. Genes that appeared in at least two out of the four top-200 gene inflammation ranked lists, as indicated by the horizontal black threshold line, and were statistically significant for fold change in both GHS and REGN cohorts were selected to be in the inflammation gene panel.

FIG. 2 (Panel C) shows a representative distribution of the number of times a given gene appeared across the six top-200 gene inflammation ranked lists, the expected distribution of the number of times a gene appears across six randomly selected lists of 200 genes, and the distribution of the number of times each gene in the selected inflammation gene panel appeared across the six top-200 gene inflammation ranked lists. The six inflammation ranked lists are one list of fold change and one of precision-recall area under the curve, comparing inflammation stage 2 and higher versus inflammation stage 0, from each of the three GHS, REGN, and Govaere cohorts.

FIG. 3 (Panels A, B, and C) shows a representative variation of fibrosis TS with fibrosis stage across the GHS, REGN, and Govaere cohorts, respectively. For each of Panels A, B, and C: i) the abscissa represents the fibrosis stage determined by histopathology; ii) the ordinate represents the fibrosis TS determined from assessing the expression of 153 genes; and iii) the N values are the number of subjects. Each dot represents the fibrosis TS for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 3 (Panels D and E) shows, within each cohort, representative values of either the precision-recall area-under-the-curve (PRAUC) or the receiver operator characteristic area-under-the-curve (ROCAUC) classification metric, respectively, from using fibrosis TSs to classify fibrosis stage F3 and higher (“positive” subjects) versus fibrosis stage F0 and F1 (“negative” subjects) (ordinate) with increasing number of genes in the gene panel studied (abscissa). From left to right along the abscissa, the gene panel is incrementally increased by one gene next in rank order of the gene panel, from a single gene to the full panel size of 153 genes. Error bars represent the 95% confidence interval of AUC calculated from stratified bootstrapped resampling with replacement. In Panel D, horizontal dotted lines represent the baseline PRAUC of the null random model for each cohort, which is the fraction of “positive” subjects out of total subjects. In Panel E, the baseline ROCAUC of the null random model is 0.5. For all three cohorts for each gene panel size, fibrosis TSs classify fibrosis stage significantly better than the null random model (p<0.001) in both PRAUC and ROCAUC.

FIG. 4 (Panels A, B, and C) shows a representative variation of inflammation TS with inflammation stage across the GHS, REGN, and Govaere cohorts, respectively. For each of Panels A, B, and C: i) the abscissa represents the inflammation stage determined by histopathology; ii) the ordinate represents the inflammation TS determined from assessing the expression of 159 genes; and iii) the N values are the number of subjects. Each dot represents the inflammation TS for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 4 (Panels D and E) shows, within each cohort, representative values of either the PRAUC or the ROCAUC classification metric, respectively, from using inflammation TSs to classify inflammation stage 2 and 3 (“positive” subjects) versus inflammation stage 0 (“negative” subjects) (ordinate) with increasing number of genes in the gene panel studied (abscissa). From left to right along the abscissa, the gene panel is incrementally increased by one gene next in rank order of the gene panel, from a single gene to the full panel size of 159 genes. Error bars represent the 95% confidence interval of AUC calculated from stratified bootstrapped resampling with replacement. In Panel D, horizontal dotted lines represent the baseline PRAUC of the null random model for each cohort, which is the fraction of “positive” subjects out of total subjects. In Panel E, the baseline ROCAUC of the null random model is 0.5. For all three cohorts for each gene panel size, inflammation transcriptomic scores classify inflammation stage significantly better than the null random model (p<0.001) in both PRAUC and ROCAUC.

FIG. 5 (Panels A and B) shows two representative methods for testing the robustness of the gene panel selection methodology by training the gene panel from a subset of the available subjects and testing the gene panel on the other subjects. Panel A shows a representative methodology of using n−1 of the available n cohorts to select the gene panel and evaluating the performance of the gene panel on the held-out cohort. Panel B shows a representative methodology of splitting each cohort into a training split and a testing split, using the training splits from each cohort to select the gene panel, and evaluating the performance of the gene panel on the testing split from each cohort.

FIG. 6 (Panels A, B, and C) shows representative variation of fibrosis TS, calculated using a gene panel derived from only the GHS and REGN cohorts, with fibrosis stage across the GHS, REGN, and Govaere cohorts, respectively. For each of Panels A, B, and C: i) the abscissa represents the fibrosis stage determined by histopathology; ii) the ordinate represents the fibrosis TS determined from assessing the expression of 195 genes; and iii) the N values are the number of subjects. Each dot represents the fibrosis TS for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 6 (Panels D and E) shows, within each cohort, representative values of either the PRAUC or the ROCAUC classification metric, respectively, from using fibrosis TSs to classify fibrosis stage F3 and higher (“positive” subjects) versus fibrosis stage F0 and F1 (“negative” subjects) (ordinate) with increasing number of genes in the gene panel studied (abscissa). From left to right along the abscissa, the gene panel is incrementally increased by one gene next in rank order of the gene panel, from a single gene to the full panel size of 195 genes. Error bars represent the 95% confidence interval of AUC calculated from stratified bootstrapped resampling with replacement. In Panel D, horizontal dotted lines represent the baseline PRAUC of the null random model for each cohort, which is the fraction of “positive” subjects out of total subjects. In Panel E, the baseline ROCAUC of the null random model is 0.5. For all three cohorts for each gene panel size, fibrosis TSs classify fibrosis stage significantly better than the null random model (p<0.001) in both PRAUC and ROCAUC.

FIG. 7 (Panels A, B, and C) shows representative variation of fibrosis TS, calculated using a gene panel derived from only the GHS and Govaere cohorts, with fibrosis stage across the GHS, REGN, and Govaere cohorts, respectively. For each of Panels A, B, and C: i) the abscissa represents the fibrosis stage determined by histopathology; ii) the ordinate represents the fibrosis TS determined from assessing the expression of 191 genes; and iii) the N values are the number of subjects. Each dot represents the fibrosis TS for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 7 (Panel D and E) shows, within each cohort, representative values of either the PRAUC or the ROCAUC classification metric, respectively, from using fibrosis TSs to classify fibrosis stage F3 and higher (“positive” subjects) versus fibrosis stage F0 and F1 (“negative” subjects) (ordinate) with increasing number of genes in the gene panel studied (abscissa). From left to right along the abscissa, the gene panel is incrementally increased by one gene next in rank order of the gene panel, from a single gene to the full panel size of 191 genes. Error bars represent the 95% confidence interval of AUC calculated from stratified bootstrapped resampling with replacement. In Panel D, horizontal dotted lines represent the baseline PRAUC of the null random model for each cohort, which is the fraction of “positive” subjects out of total subjects. In Panel E, the baseline ROCAUC of the null random model is 0.5. For all three cohorts for each gene panel size, fibrosis TSs classify fibrosis stage significantly better than the null random model (p<0.001) in both PRAUC and ROCAUC.

FIG. 8 (Panels A, B, and C) shows representative variation of fibrosis TS, calculated using a gene panel derived from only the REGN and Govaere cohorts, with fibrosis stage across the GHS, REGN, and Govaere cohorts, respectively. For each of Panels A, B, and C: i) the abscissa represents the fibrosis stage determined by histopathology; ii) the ordinate represents the fibrosis TS determined from assessing the expression of 202 genes; and iii) the N values are the number of subjects. Each dot represents the fibrosis transcriptomic score for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 8 (Panels D and E) shows, within each cohort, representative values of either the PRAUC or the ROCAUC classification metric, respectively, from using fibrosis TSs to classify fibrosis stage F3 and higher (“positive” subjects) versus fibrosis stage F0 and F1 (“negative” subjects) (ordinate) with increasing number of genes in the gene panel studied (abscissa). From left to right along the abscissa, the gene panel is incrementally increased by one gene next in rank order of the gene panel, from a single gene to the full panel size of 202 genes. Error bars represent the 95% confidence interval of AUC calculated from stratified bootstrapped resampling with replacement. In Panel D, horizontal dotted lines represent the baseline PRAUC of the null random model for each cohort, which is the fraction of “positive” subjects out of total subjects. In Panel E, the baseline ROCAUC of the null random model is 0.5. For all three cohorts for each gene panel size, fibrosis TSs classify fibrosis stage significantly better than the null random model (p<0.001) in both PRAUC and ROCAUC.

FIG. 9 (Panels A, B, C, D, E, and F) shows representative variation of fibrosis TS, calculated using a gene panel derived from only the training splits from the GHS, REGN, and Govaere cohorts, with fibrosis stage across each training and testing split from the GHS, REGN, and Govaere cohorts, respectively. For each of Panels A, B, C, D, E, and F: i) the abscissa represents the fibrosis stage determined by histopathology; ii) the ordinate represents the fibrosis TS determined from assessing the expression of 172 genes; and iii) the N values are the number of subjects. Each dot represents the fibrosis TS for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 10 (Panels A, B, C, D, and E) shows representative variation of inflammation TS, calculated using a gene panel derived from only the training splits from the GHS and REGN cohorts, with inflammation stage across each training and testing split from the GHS and REGN cohorts and the entire Govaere cohort, respectively. For each of Panels A, B, C, D, and E: i) the abscissa represents the inflammation stage determined by histopathology; ii) the ordinate represents the inflammation TS determined from assessing the expression of 150 genes; and iii) the N values are the number of subjects. Each dot represents the inflammation TS for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 11 shows a representative variation of fibrosis TS, calculated using the fibrosis gene panel, with fibrosis stage across an external cohort of 28 participants. In this figure: i) the abscissa represents the fibrosis stage determined by histopathology; ii) the ordinate represents the fibrosis TS determined from assessing the expression of 153 genes; and iii) the N values are the number of subjects. Each dot represents the fibrosis transcriptomic score for a single subject. In each box, the middle thick horizontal line represents the median, the hinges represent the first and third quartiles (of whose difference is called the inter-quartile range or IQR), the upper whisker is drawn to the largest value no larger than 1.5 times the IQR from the upper hinge, and the lower whisker is drawn to the smallest value no smaller than 1.5 times the IQR below the lower hinge.

FIG. 12 (Panels A, B, C, D, E, F, G, and H) shows representative variation of clinical biomarkers and of fibrosis TS, calculated using the fibrosis gene panel, with fibrosis stage across an external cohort of 28 participants. For each of Panels A, B, C, D, E, F, G, and H, the abscissa represents the fibrosis stage determined by histopathology. For Panel A, the ordinate represents serum measurements of alanine aminotransferase (ALT) in units of U/L. For Panel B, the ordinate represents scores from non-invasive liver transient elastography FibroScan® in units of kPa. For Panel C, the ordinate represents scores from the Enhanced Liver Fibrosis (ELF)™ algorithm. For Panel D, the ordinate represents scores from the FIB-4 algorithm. For Panel E, the ordinate represents scores from the FibroTest™ algorithm. For Panel F, the ordinate represents serum measurements of caspase-cleaved cytokeratin 18 (M30) in units of U/L. For Panel G, the ordinate represents serum measurements of total cytokeratin 18 (M65) in units of U/L. For Panel H, the ordinate represents the fibrosis TS determined from assessing the expression of 153 genes, and this panel displays the same values as in FIG. 11. For each of Panels A, B, C, D, E, F, G, and H: i) Each dot represents the measurement or score for a single subject, ii) the N values are the number of subjects, iii) the Spearman's rank correlation coefficient (rho) of each biomarker against fibrosis histopathology is indicated at the bottom of each panel, and iv) the rho value is bolded and underlined if its p value is less than 0.05. The participant with the highest fibrosis transcriptomic score within the F1 fibrosis histopathology category is indicated with a box around the dot, and measurements across the clinical biomarkers from this same participant are similarly indicated with a box around the dot across the panels.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician or a veterinarian.

As used herein, a list comprising A, B, “and/or” C provides: (i) A alone; (ii) B alone; (iii) C alone; (iv) A and B; (v) A and C; (vi) B and C; and (viii) A, B, and C. Thus, a list comprising A, B, C, . . . , and/or N has n constituents, where n is a positive integer provides all possible combinations of A, B, C, . . . N up to and including a combination of all n constituents.

The present disclosure provides methods of identifying and/or evaluating liver inflammation and/or liver fibrosis in a subject. In some embodiments, the methods comprise identifying and/or evaluating liver inflammation in a subject. In some embodiments, the methods comprise identifying and/or evaluating liver fibrosis in a subject. The methods comprise determining or having determined a subject's Transcriptome Score (TS). The TS comprises a value determined from RNA expression in a biological sample from the subject. When the subject's TS is greater than a threshold TS determined from a reference population of subjects without liver inflammation and/or without liver fibrosis in a subject, then the subject is identified as having liver inflammation and/or liver fibrosis. In some embodiments, when the subject's TS is greater than a threshold TS determined from a reference population of subjects without liver inflammation, then the subject is identified as having liver inflammation. In some embodiments, when the subject's TS is greater than a threshold TS determined from a reference population of subjects without liver fibrosis, then the subject is identified as having liver fibrosis.

In some embodiments, when the subject's TS is greater than a threshold TS determined from a reference population of subjects with liver inflammation and/or with liver fibrosis in a subject, then the subject is identified as having liver inflammation and/or liver fibrosis. In some embodiments, when the subject's TS is greater than a threshold TS determined from a reference population of subjects with liver inflammation, then the subject is identified as having liver inflammation. In some embodiments, when the subject's TS is greater than a threshold TS determined from a reference population of subjects with liver fibrosis, then the subject is identified as having liver fibrosis. In this manner, the methods can be used to distinguish the sickest subjects from a generally sick population. Thus, these methods can be used to rank disease severity.

The magnitude of any particular TS can also help characterize the degree of liver inflammation and/or liver fibrosis. For example, subject A having a TS that is twice the value of the TS from subject B may have a level of liver inflammation, for example, that is greater than the level of liver inflammation in subject B.

The present disclosure provides methods of identifying and/or evaluating liver inflammation and/or liver fibrosis in a subject. In some embodiments, the methods comprise identifying and/or evaluating liver inflammation in a subject. In some embodiments, the methods comprise identifying and/or evaluating liver fibrosis in a subject. The methods comprise determining or having determined a subject's Transcriptome Score (TS). The TS comprises a value determined from changes in RNA expression of genes of longitudinal liver samples from the subject. In some embodiments, TS comprises a value determined from changes in RNA expression across multiple biological samples from the subject. The larger a subject's TS, the larger the change in liver inflammation and/or liver fibrosis in a subject. In some embodiments, a subject's TS represents a decrease in liver inflammation and/or liver fibrosis in a subject. In some embodiments, a subject's TS represents an increase in liver inflammation and/or liver fibrosis in a subject. In this manner, the methods described herein can be used to track the progression or remission of liver inflammation and/or liver fibrosis in a subject. For example, a subject having a TS that doubles in magnitude between an earlier time point and a later time point can be considered to have a progression of, for example, liver inflammation. In contrast, a subject having a TS at a later time point that is half the TS the subject had at an earlier time point can be considered to have a remission of, for example, liver inflammation.

In some embodiments, there are two ways of calculating TS when pre-treatment and post-treatment RNA measurements are available. First, a single TS score can be obtained incorporating the pre-treatment and post-treatment measurements. Second, two different TS scores can be obtained, one derived from the pre-treatment RNA measurement, and one from post-treatment RNA measurement.

In some embodiments, the methods further comprise a therapy. When the subject's TS is greater than a threshold TS determined from a reference population of subjects without liver inflammation and/or without liver fibrosis, then the subject is administered a therapeutic agent that treats or inhibits liver inflammation and/or liver fibrosis and/or a surgery is performed on the subject. In some embodiments, when the subject's TS is greater than a threshold TS determined from a reference population of subjects without liver inflammation, then the subject is administered a therapeutic agent that treats or inhibits liver inflammation and/or a surgery is performed on the subject. In some embodiments, when the subject's TS is greater than a threshold TS determined from a reference population of subjects without liver fibrosis, then the subject is administered a therapeutic agent that treats or inhibits liver fibrosis and/or a surgery is performed on the subject.

In some embodiments, the methods further comprise a therapy. When the subject's TS at a later time point is greater than the subject's TS at an earlier time point, then the subject is administered an increased amount of a therapeutic agent that treats or inhibits liver inflammation and/or liver fibrosis and/or a surgery is performed on the subject, and/or a change in therapeutic agents is undertaken. When the subject's TS at a later time point is less than the subject's TS at an earlier time point, then the subject is administered the same amount or a decreased amount of a therapeutic agent that treats or inhibits liver inflammation and/or liver fibrosis.

In some embodiments, the methods described herein can be used to evaluate drug efficacy. The TS can either be calculated from a single timepoint from a particular subject or calculated from the difference in RNA expression from two timepoints from the same subject. For example, when the TS is calculated from two timepoints for the same subject, when a subject's TS at a later time point is greater than the subject's TS at an earlier time point, wherein the subject is receiving treatment between the two time periods with a therapeutic agent, such therapeutic agent may be evaluated for its efficacy, in this case less than desired. When a subject's TS at a later time point is less than the subject's TS at an earlier time point, wherein the subject is receiving treatment between the two time periods with a therapeutic agent, such therapeutic agent may be evaluated for its efficacy, in this case producing a desired result. The efficacy of a particular therapeutic agent can be tracked at multiple time points with a subject. For example, when the TS is calculated from the difference in RNA expression from two timepoints, wherein the subject is receiving treatment between the two time periods with a therapeutic agent, such therapeutic agent may be evaluated for its efficacy based on the TS.

In various embodiments, the therapeutic agent for treating liver inflammation and/or liver fibrosis comprises any agent effective in treating liver inflammation and/or liver fibrosis. In some embodiments, the therapeutic agent for treating liver inflammation and/or liver fibrosis comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). In some embodiments, the therapeutic agent for treating liver inflammation and/or liver fibrosis comprises an antisense RNA. In some embodiments, the therapeutic agent for treating liver inflammation and/or liver fibrosis comprises an siRNA. In some embodiments, the therapeutic agent for treating liver inflammation and/or liver fibrosis comprises an shRNA. In some embodiments, the therapeutic agent for treating liver inflammation and/or liver fibrosis comprises an inhibitory nucleic acid molecule directed to HSD17B13. In some embodiments, the therapeutic agent for treating liver inflammation and/or liver fibrosis comprises an HSD117B13 inhibitor. In some embodiments, the therapeutic agent for treating liver inflammation and/or liver fibrosis comprises an inhibitory nucleic acid molecule directed to PNPLA3. In some embodiments, the therapeutic agent for treating liver inflammation and/or liver fibrosis comprises an PNPLA3 inhibitor, including, but not limited to momelotinib. In some embodiments, the therapeutic agent for treating liver inflammation and/or liver fibrosis comprises an inhibitory nucleic acid molecule directed to CIDEB. In some embodiments, the therapeutic agent for treating liver inflammation and/or liver fibrosis comprises an CIDEB inhibitor.

In some embodiments, the HSD17B13 inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of a HSD17B13 mRNA. In some embodiments, the antisense RNA, siRNA, or shRNA hybridizes to a sequence within a HSD17B13 genomic nucleic acid molecule or mRNA molecule and decreases expression of the HSD17B13 polypeptide in a cell in the subject. In some embodiments, the HSD17B13 inhibitor comprises an antisense RNA that hybridizes to a HSD17B13 genomic nucleic acid molecule or mRNA molecule and decreases expression of the HSD17B13 polypeptide in a cell in the subject. In some embodiments, the HSD17B13 inhibitor comprises an siRNA that hybridizes to a HSD17B13 genomic nucleic acid molecule or mRNA molecule and decreases expression of the HSD17B13 polypeptide in a cell in the subject. In some embodiments, the HSD17B13 inhibitor comprises an shRNA that hybridizes to a HSD17B13 genomic nucleic acid molecule or mRNA molecule and decreases expression of the HSD17B13 polypeptide in a cell in the subject.

The inhibitory nucleic acid molecules described herein can be targeted to various HSD17B13 transcripts. For example, the inhibitory nucleic acid molecules described herein can be targeted to the HSD17B13 transcripts (derived from chromosome 4; Ensembl Gene ID=ENSG00000170509.8; hgnc symbol=HSD17B13).

In some embodiments, the HSD17B13 inhibitor is a small molecule. Numerous HSD17B13 inhibitors are described in, for example, PCT Publications WO2019/183329, WO2019/183164, and WO2020/061177. In some embodiments, the HSD17B13 inhibitor is an antibody. In some embodiments, the HSD17B13 inhibitor comprises an inhibitory nucleic acid molecule, such as, for example an antisense nucleic acid molecule, an siRNA, or an shRNA. Additional examples of HSD17B13 inhibitors include, but are not limited to ARO-HSD or ALN-HSD.

In some embodiments, the PNPLA3 inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of a PNPLA3 mRNA. In some embodiments, the antisense RNA, siRNA, or shRNA hybridizes to a sequence within a PNPLA3 genomic nucleic acid molecule or mRNA molecule and decreases expression of the PNPLA3 polypeptide in a cell in the subject. In some embodiments, the PNPLA3 inhibitor comprises an antisense RNA that hybridizes to a PNPLA3 genomic nucleic acid molecule or mRNA molecule and decreases expression of the PNPLA3 polypeptide in a cell in the subject. In some embodiments, the PNPLA3 inhibitor comprises an siRNA that hybridizes to a PNPLA3 genomic nucleic acid molecule or mRNA molecule and decreases expression of the PNPLA3 polypeptide in a cell in the subject. In some embodiments, the PNPLA3 inhibitor comprises an shRNA that hybridizes to a PNPLA3 genomic nucleic acid molecule or mRNA molecule and decreases expression of the PNPLA3 polypeptide in a cell in the subject.

The inhibitory nucleic acid molecules described herein can be targeted to various PNPLA3 transcripts. For example, the inhibitory nucleic acid molecules described herein can be targeted to the PNPLA3 transcripts (derived from chromosome 22; Ensembl Gene ID=ENSG00000100344.11; hgnc symbol=PNPLA3).

In some embodiments, the PNPLA3 inhibitor is a small molecule. In some embodiments, the PNPLA3 inhibitor is an antibody. In some embodiments, the PNPLA3 inhibitor comprises an inhibitory nucleic acid molecule, such as, for example an antisense nucleic acid molecule, an siRNA, or an shRNA. An exemplary PNPLA3 inhibitor is AZD2693.

In some embodiments, the CIDEB inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a CIDEB mRNA. In some embodiments, the antisense RNA, siRNA, or shRNA hybridizes to a sequence within a CIDEB genomic nucleic acid molecule or mRNA molecule and decreases expression of the CIDEB polypeptide in a cell in the subject. In some embodiments, the CIDEB inhibitor comprises an antisense RNA that hybridizes to a CIDEB genomic nucleic acid molecule or mRNA molecule and decreases expression of the CIDEB polypeptide in a cell in the subject. In some embodiments, the CIDEB inhibitor comprises an siRNA that hybridizes to a CIDEB genomic nucleic acid molecule or mRNA molecule and decreases expression of the CIDEB polypeptide in a cell in the subject. In some embodiments, the CIDEB inhibitor comprises an shRNA that hybridizes to a CIDEB genomic nucleic acid molecule or mRNA molecule and decreases expression of the CIDEB polypeptide in a cell in the subject. Specific antisense and siRNA molecules are disclosed in, for example, PCT Publication WO 2022/140624.

The inhibitory nucleic acid molecules described herein can be targeted to various CIDEB transcripts. For example, the inhibitory nucleic acid molecules described herein can be targeted to the CIDEB transcripts (derived from chromosome 14; Ensembl Gene ID=ENSG00000136305; hgnc symbol=CIDEB; from top to bottom=Transcript A, Transcript B, Transcript C, Transcript D, Transcript E, and Transcript F) in the Table below.

| Ensembl | Transcript | Transcript | | | |
|---|---|---|---|---|---|
| Transcript id | Start | End | Name | Coordinates | Length |
| ENST00000258807 | 24305187 | 24311422 | CIDEB_258807 | chr14:24311422- | 6235 |
| | | | | 24305187 | |
| ENST00000336557 | 24305187 | 24311395 | CIDEB_336557 | chr14:24311395- | 6208 |
| | | | | 24305187 | |
| ENST00000554411 | 24305096 | 24308263 | CIDEB_554411 | chr14:24308263- | 3167 |
| | | | | 24305096 | |
| ENST00000555471 | 24310087 | 24310718 | CIDEB_555471 | chr14:24310718- | 631 |
| | | | | 24310087 | |
| ENST00000555817 | 24310799 | 24311430 | CIDEB_555817 | chr14:24311430- | 631 |
| | | | | 24310799 | |
| ENST00000556756 | 24305606 | 24306461 | CIDEB_556756 | chr14:24306461- | 855 |
| | | | | 24305606 | |

Additional CIDEB transcripts include, but are not limited to those of the following Ensembl Gene IDs=ENST00000555471, ENST00000555817, ENST00000556756, ENST00000258807, ENST00000336557, and ENST00000554411.

In some embodiments, the CIDEB inhibitor is a small molecule. In some embodiments, the CIDEB inhibitor is an antibody. In some embodiments, the CIDEB inhibitor comprises an inhibitory nucleic acid molecule, such as, for example an antisense nucleic acid molecule, an siRNA, or an shRNA.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (1), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

### Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the therapeutic agent for treating liver inflammation and/or liver fibrosis includes, but is not limited to, obeticholic acid, GS-9674, simtuzumab, GS-4997, NDI-010976, GFT505/elafibranor, aramchol, cenicriviroc, GR-MD-02, TD139, SHP626, PXS4728A, and RP103-cysteamine bitartrate.

Additional examples of liver disease therapeutic agents include, but are not limited to, disulfiram, naltrexone, acamprosate, prednisone, azathioprine, penicillamine, trientine, deferoxamine, ciprofloxacin, norofloxacin, ceftriaxone, ofloxacin, amoxicillin-clavulanate, phytonadione, bumetanide, furosemide, hydrochlorothiazide, chlorothiazide, amiloride, triamterene, spironolactone, octreotide, atenolol, metoprolol, nadolol, propranolol, timolol, and carvedilol.

Additional examples of liver disease therapeutic agents (e.g., for use in chronic hepatitis C treatment) include, but are not limited to, ribavirin, paritaprevir, OLYSIO© (simeprevir), grazoprevir, ledipasvir, ombitasvir, elbasvir, DAKLINZA® (daclatasvir), dasabuvir, ritonavir, sofosbuvir, velpatasvir, voxilaprevir, glecaprevir, pibrentasvir, peginterferon alfa-2a, peginterferon alfa-2b, and interferon alfa-2b.

Additional examples of liver disease therapeutic agents (e.g., for use in nonalcoholic fatty liver disease) include, but are not limited to, weight loss inducing agents such as orlistat or sibutramine; insulin sensitizing agents such as thiazolidinediones (TZDs), metformin, and meglitinides; lipid lowering agents such as statins, fibrates, and omega-3 fatty acids; antioxidants such as, vitamin E, betaine, N-Acetyl-cysteine, lecithin, silymarin, and beta-carotene; anti TNF agents such as pentoxifylline; probiotics, such as VSL #3; and cytoprotective agents such as ursodeoxycholic acid (UDCA). Other suitable treatments include ACE inhibitors/ARBs, oligofructose, and Incretin analogs.

Additional examples of liver disease therapeutic agents (e.g., for use in NASH) include, but are not limited to, OCALIVA® (obeticholic acid), Selonsertib, Elafibranor, Cenicriviroc, GR_MD_02, MGL_3196, IMM124E, ARAMCHOL™ (arachidyl amido cholanoic acid), GS0976, Emricasan, Volixibat, NGM282, GS9674, Tropifexor, MN_001, LMB763, BI_1467335, MSDC_0602, PF_05221304, DF102, Saroglitazar, BMS986036, Lanifibranor, Semaglutide, Nitazoxanide, GRI_0621, EYP001, VK2809, Nalmefene, LIK066, MT_3995, Elobixibat, Namodenoson, Foralumab, SAR425899, Sotagliflozin, EDP_305, Isosabutate, Gemcabene, TERN_101, KBP_042, PF_06865571, DUR928, PF_06835919, NGM313, BMS_986171, Namacizumab, CER_209, ND_L02_s0201, RTU_1096, DRX_065, IONIS_DGAT2Rx, INT_767, NC_001, Seladepar, PXL770, TERN_201, NV556, AZD2693, SP_1373, VK0214, Hepastem, TGFTX4, RLBN1127, GKT_137831, RYI_018, CB4209-CB4211, and JH_0920.

Additional examples of therapeutic agents that treat or inhibit liver disease include, but are not limited to, a corticosteroid, ursodeoxycholic acid, methotrexate (MTX), a peroxisome proliferator-activated receptor (PPAR) gamma ligand, colchicine, an angiotensin receptor blocker, and pirfenidone.

The TS comprises quantification (i.e., a measurement based on potentially many RNAs) of RNA/transcript expression of at least one gene in a biological sample from a subject. As used herein, “gene” is meant to also capture non-coding genes/biotypes (e.g., long non-coding RNAs). In some embodiments, the TS comprises quantification of an RNA expression level(s) of at least one gene in a biological sample from a subject. In some embodiments, the TS comprises quantification of an RNA expression level(s) of at least 10 genes. In some embodiments, the TS comprises quantification of an RNA expression level(s) of at least 20 genes. In some embodiments, the TS comprises quantification of an RNA expression level(s) of a level of at least 30 genes. In some embodiments, the TS comprises quantification of an RNA expression level(s) of at least 40 genes. In some embodiments, the TS comprises quantification of an RNA expression level(s) of at least 50 genes. In some embodiments, the TS comprises quantification of an RNA expression level(s) of at least 60 genes. In some embodiments, the TS comprises quantification of an RNA expression level(s) of at least 70 genes. In some embodiments, the TS comprises quantification of an RNA expression level(s) of at least 80 genes. In some embodiments, the TS comprises quantification of an RNA expression level(s) of at least 90 genes. In some embodiments, the TS comprises quantification of an RNA expression level(s) of at least 100 genes. In some embodiments, the TS comprises quantification of an RNA expression level(s) of at least 125 genes. In some embodiments, the TS comprises quantification of an RNA expression level(s) of at least 150 genes. In some embodiments, the TS comprises quantification of an RNA expression level(s) of at least 175 genes. In some embodiments, the TS comprises quantification of an RNA expression level(s) of at least 200 genes. In some embodiments, the TS comprises quantification of an RNA expression level(s) of at least 300 genes. In some embodiments, the TS comprises quantification of an RNA expression level(s) of at least 400 genes. In some embodiments, the TS comprises quantification of an RNA expression level(s) of at least 500 genes. In some embodiments, the TS comprises quantification of an RNA expression level(s) of at least 600 genes. In some embodiments, the TS comprises quantification of an RNA expression level(s) of at least 700 genes. In some embodiments, the TS comprises quantification of an RNA expression level(s) of at least 800 genes. In some embodiments, the TS comprises quantification of an RNA expression level(s) of at least 900 genes. In some embodiments, the TS comprises quantification of an RNA expression level(s) of at least 1,000 genes. In some embodiments, the TS comprises quantification of an RNA expression level of at least 5,000 genes. In some embodiments, the TS comprises quantification of an RNA expression level of at least 10,000 genes. In some embodiments, the TS comprises quantification of an RNA expression level of at least 15,000 genes. In some embodiments, the TS comprises quantification of an RNA expression level of at least 20,000 genes. In some embodiments, the TS comprises quantification of an RNA expression level of at least 25,000 genes. In some embodiments, the TS comprises quantification of an RNA expression level of at least 30,000 genes.

In some embodiments, the at least one gene comprises a protein-coding gene, a non-coding gene, a long non-coding RNA, a mitochondrial rRNA, a mitochondrial tRNA, an rRNA, a ribozyme, a B-cell receptor subunit constant gene, and/or a T-cell receptor subunit constant gene, or any combination thereof. In some embodiments, the at least one gene comprises a protein-coding gene. In some embodiments, the at least one gene comprises a non-coding gene. In some embodiments, the at least one gene comprises a long non-coding RNA. In some embodiments, the at least one gene comprises a mitochondrial rRNA. In some embodiments, the at least one gene comprises a mitochondrial tRNA. In some embodiments, the at least one gene comprises an rRNA. In some embodiments, the at least one gene comprises a ribozyme. In some embodiments, the at least one gene comprises a B-cell receptor subunit constant gene. In some embodiments, the at least one gene comprises a T-cell receptor subunit constant gene.

In some embodiments, the gene whose RNA expression is included in the reference population and/or diseased population, and which may be included in the panel of genes whose RNA expression is examined in the biological sample of the subject, is derived from at least one dataset paired with histopathological data. In some embodiments, the genes included in the panel are derived from a plurality of datasets. In some embodiments, the dataset is that of Geisinger Health System MyCode Community Health Initiative cohort, in which some samples were sequenced at Geisinger (referred to as “GHS cohort”) and in which some samples were sequenced at Regeneron (referred to as “REGN cohort”). In some embodiments, the dataset is that disclosed in Govaere et al., Sci. Transl. Med., 2019, 12, eaba4448 (referred to as “Govaere cohort”). In some embodiments, only samples with paired histopathology showing steatosis were used in analysis. In some embodiments, fibrosis and lobular inflammation (sometimes referred to herein as inflammation) on histopathology of the samples is scored following the NASH Clinical Research Network system. In some embodiments, the fibrosis is scored as: no fibrosis, 0; portal fibrosis without septa, 1; portal fibrosis with septa, 2; bridging fibrosis, 3; between bridging fibrosis and cirrhosis, 3-4; and cirrhosis, 4. In some embodiments, the inflammation is scored as: no foci, 0; <2 foci/200×, 1; 2-4 foci/200×, 2; and >4 foci/200×, 3.

In some embodiments, the liver inflammation associated with a particular gene comprises inflammation associated with alcohol abuse, an alpha-1 antitrypsin deficiency, an autoimmune reaction, a decrease of a blood flow to the liver, a drug, a toxin, hemochromatosis, obstructive jaundice, a viral infection, Wilson's disease, or nonalcoholic fatty liver disease. In some embodiments, the viral infection comprises a hepatitis A viral infection, a hepatitis B viral infection, a hepatitis C viral infection, a hepatitis D viral infection, or a hepatitis E viral infection. In some embodiments, the nonalcoholic fatty liver disease comprises nonalcoholic steatohepatitis.

In some embodiments, the liver fibrosis associated with a particular gene comprises fibrosis associated with alcohol abuse, fibrosis associated with a hepatitis C infection, fibrosis associated with nonalcoholic fatty liver disease, or cirrhosis. In some embodiments, the nonalcoholic fatty liver disease comprises nonalcoholic steatohepatitis.

The methods disclosed herein can be used, for example, to monitor therapeutic efficacy and/or progress of disease progression. For example, a subject who will be undergoing treatment for liver inflammation and/or liver fibrosis, can be assessed pre-treatment for their TS (such as at a single time point). The subject's TS can then be assessed at various stages of treatment (and/or post-treatment; such as at more than a single time point) with a therapeutic agent (or other treatment protocol) to determine whether the therapeutic agent (or other treatment protocol) is sufficiently working as desired. If not, a different therapeutic agent (or other treatment protocol) can be employed. For example, regression of liver diseases, such as NASH, can be assessed by monitoring a subject's TS throughout the treatment and post-treatment stages. In some embodiments, the state of NASH in a subject may stay the same, regress, or progress, and such NASH states can be assessed by monitoring the subjects' TS.

In some embodiments, the subject may comprise a CIDEB variant nucleic acid molecule comprising: 14:24305635:A:AGTAG, 14:24305641:A:C, 14:24305650:G:A, 14:24305657:C:A, 14:24305662:G:T, 14:24305667:T:C, 14:24305671:C:A, 14:24305671:C:G, 14:24305701:A:T, 14:24305709:C:T, 14:24305718:A:G, 14:24305721:T:C, 14:24305728:G:GGCCTT, 14:24305743:T:C, 14:24305948:T:C, 14:24305966:C:T, 14:24305974:T:C, 14:24305980:TCA:T, 14:24305988:C:T, 14:24306014:C:T, 14:24306034:A:C, 14:24306041:C:G, 14:24306044:G:A, 14:24306047:G:A, 14:24306051:T:G, 14:24306064:T:C, 14:24306074:A:G, 14:24306077:G:C, 14:24306082:A:G, 14:24306083:T:A, 14:24306095:G:A, 14:24306122:A:G, 14:24306134:C:G, 14:24306373:C:G, 14:24306379:T:C, 14:24306382:G:A, 14:24306383:G:T, 14:24306426:T:G, 14:24306437:C:G, 14:24306439:G:C, 14:24306442:A:G, 14:24306444:A:G, 14:24306457:C:T, 14:24306463:C:T, 14:24306469:C:T, 14:24306480:A:G, 14:24306486:A:C, 14:24306504:A:G, 14:24306519:A:G, 14:24307382:G:C, 14:24307405:A:G, 14:24307417:A:T, 14:24307421:T:A, 14:24307441:C:A, 14:24307444:A:C, 14:24307444:A:G, 14:24307450:C:CGCTG, 14:24307461:TG:T, 14:24307469:AG:A, 14:24307474:C:T, 14:24307475:A:G, 14:24307833:G:C, 14:24307851:T:TAC, 14:24306426:T:C, 14:24307849:G:C, 14:24307448:G:T, 14:24305671:C:T, 14:24305663:C:T, 14:24305686:C:G, 14:24307829:A:C, 14:24307818:CTGAG:C, 14:24307856:C:T, 14:24306423:T:C, 14:24306061:AC:A, 14:24307390:C:T, 14:24306382:G:T, 14:24306373:C:T, 14:24305733:T:C, 14:24307858:T:C, 14:24306387:C:T, 14:24305637:T:C, 14:24306062:C:T, 14:24307853:C:G, 14:24307450:C:G, 14:24306052:TG:T, 14:24305673:G:A, 14:24306043:C:T, 14:24307834:G:A, 14:24306417:C:T, 14:24307451:G:A, 14:24307436:A:C, 14:24305953:ACTTT:A, 14:24306489:G:T, 14:24307441:C:T, 14:24306375:C:T, 14:24305657:C:G, 14:24306427:C:T, 14:24306524:C:T, 14:24307516:C:A, 14:24307840:G:C, 14:24307501:A:G, 14:24305968:A:C, 14:24305986:C:T, 14:24307441:C:G, 14:24307459:G:T, 14:24306017:T:A, 14:24307424:G:A, 14:24306072:G:T, 14:24307423:C:T, 14:24307450:C:T, 14:24306420:G:A, 14:24307454:G:A, 14:24305653:C:T, 14:24307442:G:A, 14:24306002:C:T, 14:24306076:C:T, 14:24305664:C:T, 14:24305961:TG:T, 14:24305706:A:G, 14:24305946:C:T, 14:24306455:G:C, 14:24307468:G:A, 14:24307825:A:C, 14:24306110:G:A, 14:24305710:C:T, 14:24307483:C:T, 14:24306459:A:G, 14:24305754:C:T, 14:24305650:G:C, 14:24305691:C:T, 14:24306508:G:C, 14:24306039:G:T, 14:24306139:T:C, 14:24306391:T:C, 14:24306373:C:A, 14:24307498:C:T, 14:24307415:G:A, 14:24306138:CTG:C, 14:24307453:T:C, 14:24305692:G:A, 14:24305683:C:G, 14:24307484:G:A, 14:24307385:C:T, 14:24306519:A:T, 14:24307839:A:C, 14:24305965:C:T, 14:24305988:CAT:C, 14:24306087:C:G, 14:24307439:C:T, 14:24307477:A:C, 14:24306436:G:T, 14:24306507:A:G, 14:24307397:C:T, 14:24307495:G:A, 14:24306034:A:T, 14:24306013:G:A, 14:24307381:A:G, 14:24306383:G:C, 14:24305638:A:G, 14:24307420:G:A, 14:24306020:C:T, 14:24306470:A:C, 14:24307435:C:T, 14:24306469:C:G, 14:24306451:C:T, 14:24306403:G:A, 14:24307515:C:G, 14:24307489:A:G, 14:24307414:C:T, 14:24306483:A:G, 14:24305755:G:A, 14:24305766:C:T, 14:24306064:T:G, 14:24307516:C:G, 14:24305766:C:G, 14:24306489:G:A, 14:24306097:T:C, 14:24305763:T:G, 14:24307447:G:A, 14:24307402:G:A, 14:24305972:C:G, 14:24306423:T:G, 14:24305974:T:TG, 14:24307411:T:C, 14:24306121:T:C, 14:24307516:C:T, 14:24306424:C:T, 14:24306039:G:C, 14:24307853:C:A, 14:24306388:A:G, 14:24305990:T:C, 14:24307822:G:GT, 14:24305640:G:A, 14:24307418:T:C, 14:24305758:G:C, 14:24306131:C:T, 14:24305953:A:G, 14:24305730:C:A, 14:24306418:A:G, 14:24306059:AC:A, 14:24307842:G:A, 14:24307837:T:G, 14:24306095:G:T, 14:24306109:C:T, 14:24307822:G:A, 14:24306077:G:A, 14:24307824:A:T, 14:24306080:C:T, 14:24305649:C:T, 14:24306433:G:GA, 14:24306420:G:C, 14:24305658:T:G, 14:24306472:C:T, 14:24307412:TC:T, 14:24306062:C:A, 14:24306044:G:C, 14:24306047:G:T, 14:24306126:CAG:C, 14:24306449:C:G, 14:24307391:G:A, or 14:24307857:A:C, according to GRCh38/hg38 human genome assembly coordinates.

In some embodiments, the subject may comprise a PNPLA3 variant nucleic acid molecule encoding a PNPLA3 Ile148Met polypeptide or a PNPLA3 Ile144Met polypeptide. The detection or determination of the presence of PNPLA3 variant nucleic acid molecules is described in, for example, U.S. Pat. No. 10,961,583.

In some embodiments, the at least one gene in the fibrosis panel comprises at least one of STMN2, FAP, ITGBL1, MOXD1, COL10A1, NALCN, SCTR, EFEMPI, CLIC6, MMP7, THY1, LOXL1, MDFI, LTBP2, VTCN1, LUM, CLDN11, CFAP221, CFTR, DCDC2, EPCAM, ADRA2A, LAMC3, AEBP1, PAPLN, RASL11B, CDH6, PTGDS, LOXL4, BHLHE22, CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, SNAP25, GJA5, UBD, DTNA, LEF1, THBS2, PLCXD3, CTHRC1, SUSD2, SMOC2, SOD3, SPON1, PDZK11P1, F3, MMP2, MFAP2, C7, CKMT2, CLDN10, CXCL6, AKR1B10, PHLDA3, COMP, CD40LG, PTK7, CCDC80, SEZ6L2, COL16A1, AQP1, GSN, GEM, NELL2, PAQR5, TYMS, LXN, TRAC, PLPP4, AP1M2, GPRC5B, VEPH1, DPPA4, RGS4, SPP1, EGFLAM, OLR1, MGP, SLC7A6, BEX2, SIT1, ANO9, DPYSL3, CD1E, ANTXR1, BOC, LAYN, PDGFD, CCL21, HKDC1, CD5, CCL19, GPC3, OMG, FBLN2, TRAT1, SEMA3G, KRT7, ANKRD29, COL5A1, IGLC3, UBASH3A, PDGFA, NFASC, CCR6, LGR6, WFDC2, NPNT, VWF, MUC6, COL1A2, SLAMF1, IGHG1, CXCR3, F13A1, TACSTD2, SIRPG, STMN3, IGFBP7, CXCL1, CCR2, CCDC146, CTSK, COL1A1, HSPB2, RRAD, COL3A1, BACE2, ZMAT3, PCYOX1L, EEF1A2, CHI3L1, FXYD2, CCL20, CH25H, MAT1A, SEPTIN8, and/or CXCL8. In some embodiments, the at least one gene comprises at least 10 of these genes. In some embodiments, the at least one gene comprises at least 20 of these genes. In some embodiments, the at least one gene comprises at least 30 of these genes. In some embodiments, the at least one gene comprises at least 40 of these genes. In some embodiments, the at least one gene comprises at least 50 of these genes. In some embodiments, the at least one gene comprises at least 60 of these genes. In some embodiments, the at least one gene comprises at least 70 of these genes. In some embodiments, the at least one gene comprises at least 80 of these genes. In some embodiments, the at least one gene comprises at least 90 of these genes. In some embodiments, the at least one gene comprises at least 100 of these genes. In some embodiments, the at least one gene comprises at least 125 of these genes. In some embodiments, the at least one gene comprises at least 150 of these genes.

In some embodiments, the at least one gene in the fibrosis panel comprises at least one of: i) STMN2, FAP, ITGBL1, MOXD1, COL10A1, and/or NALCN. In some embodiments, the at least one gene comprises any two, any three, any four, any five, or all six of the genes.

In some embodiments, the at least one gene in the fibrosis panel further comprises (in addition to i) above) at least one of: ii) SCTR, EFEMPI, CLIC6, MMP7, THY1, and/or LOXL1. In some embodiments, the at least one gene comprises any two, any three, any four, any five, or all six of the genes.

In some embodiments, the at least one gene in the fibrosis panel further comprises (in addition to i) and/or ii) above) at least one of: iii) MDFI, LTBP2, VTCN1, LUM, CLDN11, and/or CFAP221. In some embodiments, the at least one gene comprises any two, any three, any four, any five, or all six of the genes.

In some embodiments, the at least one gene in the fibrosis panel further comprises (in addition to i) and/or ii) and/or iii) above) at least one of: iv) CFTR, DCDC2, EPCAM, ADRA2A, LAMC3, and/or AEBP1. In some embodiments, the at least one gene comprises any two, any three, any four, any five, or all six of the genes.

In some embodiments, the at least one gene in the fibrosis panel further comprises (in addition to i) and/or ii) and/or iii) and/or iv) above) at least one of: v) PAPLN, RASL11B, CDH6, PTGDS, LOXL4, and/or BHLHE22. In some embodiments, the at least one gene comprises any two, any three, any four, any five, or all six of the genes.

In some embodiments, the at least one gene in the fibrosis panel further comprises (in addition to i) and/or ii) and/or iii) and/or iv) and/or v) above) at least one of: vi) CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, and/or SNAP25. In some embodiments, the at least one gene comprises any two, any three, any four, any five, any six, any seven, any eight, any nine, any ten, any eleven, any twelve, any thirteen, any fourteen, any fifteen, any sixteen, any seventeen, or all eighteen of the genes.

In some embodiments, the at least one gene in the fibrosis panel in the biological sample from the subject is upregulated compared to the corresponding gene in the reference population of subjects without liver fibrosis. In some embodiments, the at least one gene in the biological sample from the subject that is upregulated compared to the corresponding gene in the reference population comprises at least one of STMN2, FAP, ITGBL1, MOXD1, COL10A1, NALCN, SCTR, EFEMPI, CLIC6, MMP7, THY1, LOXL1, MDFI, LTBP2, VTCN1, LUM, CLDN11, CFAP221, CFTR, DCDC2, EPCAM, ADRA2A, LAMC3, AEBP1, PAPLN, RASL11B, CDH6, PTGDS, LOXL4, BHLHE22, CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, SNAP25, GJA5, UBD, DTNA, LEF1, THBS2, PLCXD3, CTHRC1, SUSD2, SMOC2, SOD3, SPON1, PDZK1IP1, F3, MMP2, MFAP2, C7, CKMT2, CLDN10, CXCL6, AKR1B10, PHLDA3, COMP, CD40LG, PTK7, CCDC80, SEZ6L2, COL16A1, AQP1, GSN, GEM, NELL2, PAQR5, TYMS, LXN, TRAC, PLPP4, AP1M2, GPRC5B, VEPH1, RGS4, SPP1, OLR1, MGP, SLC7A6, BEX2, SIT1, ANO9, DPYSL3, CD1E, ANTXR1, BOC, LAYN, PDGFD, CCL21, HKDC1, CD5, CCL19, GPC3, OMG, FBLN2, TRAT1, SEMA3G, KRT7, ANKRD29, COL5A1, IGLC3, UBASH3A, PDGFA, NFASC, CCR6, LGR6, WFDC2, NPNT, VWF, MUC6, COL1A2, SLAMF1, IGHG1, CXCR3, F13A1, TACSTD2, SIRPG, STMN3, IGFBP7, CXCL1, CCR2, CCDC146, CTSK, COL1A1, HSPB2, RRAD, COL3A1, BACE2, ZMAT3, PCYOX1L, EEF1A2, CHI3L1, FXYD2, CCL20, CH25H, SEPTIN8, and/or CXCL8. In some embodiments, at least 10 of these genes are upregulated compared to the corresponding genes in the reference population. In some embodiments, at least 20 of these genes are upregulated compared to the corresponding genes in the reference population. In some embodiments, at least 30 of these genes are upregulated compared to the corresponding genes in the reference population. In some embodiments, at least 40 of these genes are upregulated compared to the corresponding genes in the reference population. In some embodiments, at least 50 of these genes are upregulated compared to the corresponding genes in the reference population. In some embodiments, at least 60 of these genes are upregulated compared to the corresponding genes in the reference population. In some embodiments, at least 70 of these genes are upregulated compared to the corresponding genes in the reference population. In some embodiments, at least 80 of these genes are upregulated compared to the corresponding genes in the reference population. In some embodiments, at least 90 of these genes are upregulated compared to the corresponding genes in the reference population. In some embodiments, at least 100 of these genes are upregulated compared to the corresponding genes in the reference population. In some embodiments, at least 120 of these genes are upregulated compared to the corresponding genes in the reference population. In some embodiments, at least 140 of these genes are upregulated compared to the corresponding genes in the reference population.

In some embodiments, the at least one gene in the fibrosis panel in the biological sample from the subject is downregulated compared to the corresponding gene in the reference population of subjects without liver fibrosis. In some embodiments, the at least one gene in the biological sample from the subject that is downregulated compared to the corresponding gene in the reference population comprises at least one of DPPA4, EGFLAM, and/or MAT1A. In some embodiments, at least any 2, or all 3 of these genes are downregulated compared to the corresponding genes in the reference population.

In some embodiments, the fibrosis panel, which can be examined in regard to a biological sample from a subject, comprises at least one of STMN2, FAP, ITGBL1, MOXD1, COL10A1, NALCN, SCTR, EFEMPI, CLIC6, MMP7, THY1, LOXL1, MDFI, LTBP2, VTCN1, LUM, CLDN11, CFAP221, CFTR, DCDC2, EPCAM, ADRA2A, LAMC3, AEBP1, PAPLN, RASL11B, CDH6, PTGDS, LOXL4, BHLHE22, CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, SNAP25, GJA5, UBD, DTNA, LEF1, THBS2, PLCXD3, CTHRC1, SUSD2, SMOC2, SOD3, SPON1, PDZK1IP1, F3, MMP2, MFAP2, C7, CKMT2, CLDN10, CXCL6, AKR1B10, PHLDA3, COMP, CD40LG, PTK7, CCDC80, SEZ6L2, COL16A1, AQP1, GSN, GEM, NELL2, PAQR5, TYMS, LXN, TRAC, PLPP4, AP1M2, GPRC5B, VEPH1, DPPA4, RGS4, SPP1, EGFLAM, OLR1, MGP, SLC7A6, BEX2, SIT1, ANO9, DPYSL3, CD1E, ANTXR1, BOC, LAYN, PDGFD, CCL21, HKDC1, CD5, CCL19, GPC3, OMG, FBLN2, TRAT1, SEMA3G, KRT7, ANKRD29, COL5A1, IGLC3, UBASH3A, PDGFA, NFASC, CCR6, LGR6, WFDC2, NPNT, VWF, MUC6, COL1A2, SLAMF1, IGHG1, CXCR3, F13A1, TACSTD2, SIRPG, STMN3, IGFBP7, CXCL1, CCR2, CCDC146, CTSK, COL1A1, HSPB2, RRAD, COL3A1, BACE2, ZMAT3, PCYOX1L, EEF1A2, CHI3L1, FXYD2, CCL20, CH25H, MAT1A, SEPTIN8, and/or CXCL8. In some embodiments, at least one of the genes of the fibrosis panel are upregulated in connection with a disease. In some embodiments, at least one of the genes hereinbefore set forth in this paragraph are upregulated in NASH except for at least one of DPPA4, EGFLAM, and/or MAT1A. In some embodiments, at least one of the genes of the fibrosis panel are downregulated in connection with a disease. In some embodiments, at least one of the genes of the fibrosis panel are downregulated in NASH. In some embodiments, at least one of the genes of the fibrosis panel are downregulated in NASH comprises at least one of DPPA4, EGFLAM, and/or MAT1A.

In some embodiments, the fibrosis panel comprises at least one of STMN2, FAP, ITGBL1, MOXD1, COL10A1, NALCN, SCTR, EFEMPI, CLIC6, MMP7, THY1, LOXL1, MDFI, LTBP2, VTCN1, LUM, CLDN11, CFAP221, CFTR, DCDC2, EPCAM, ADRA2A, LAMC3, AEBP1, PAPLN, RASL11B, CDH6, PTGDS, LOXL4, BHLHE22, CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, SNAP25, GJA5, UBD, DTNA, LEF1, THBS2, PLCXD3, CTHRC1, SUSD2, SMOC2, SOD3, SPON1, PDZK1IP1, F3, MMP2, MFAP2, C7, CKMT2, CLDN10, CXCL6, AKR1B10, PHLDA3, COMP, CD40LG, PTK7, CCDC80, SEZ6L2, COL16A1, AQP1, GSN, GEM, NELL2, PAQR5, TYMS, LXN, TRAC, PLPP4, AP1M2, GPRC5B, VEPH1, DPPA4, RGS4, SPP1, EGFLAM, OLR1, MGP, SLC7A6, BEX2, SIT1, ANO9, DPYSL3, CD1E, ANTXR1, BOC, LAYN, PDGFD, CCL21, HKDC1, CD5, CCL19, GPC3, OMG, FBLN2, TRAT1, SEMA3G, KRT7, ANKRD29, COL5A1, IGLC3, UBASH3A, PDGFA, NFASC, CCR6, LGR6, WFDC2, NPNT, VWF, MUC6, COL1A2, SLAMF1, IGHG1, CXCR3, F13A1, TACSTD2, SIRPG, STMN3, IGFBP7, CXCL1, CCR2, CCDC146, CTSK, COL1A1, HSPB2, RRAD, COL3A1, BACE2, ZMAT3, PCYOX1L, EEF1A2, CHI3L1, FXYD2, CCL20, CH25H, MAT1A, SEPTIN8, and/or CXCL8. In some embodiments, at least one of the genes of the fibrosis panel are upregulated in connection with a disease. In some embodiments, at least one of the genes hereinbefore set forth in this paragraph are upregulated in NASH except for at least one of DPPA4, EGFLAM, and/or MAT1A. In some embodiments, at least one of the genes of the fibrosis panel are downregulated in connection with a disease. In some embodiments, the genes of the fibrosis panel are downregulated in NASH. In some embodiments, at least one of the genes of the fibrosis panel are downregulated in NASH comprises at least one of DPPA4, EGFLAM, and/or MAT1A.

In some embodiments, the fibrosis panel comprises at least one of STMN2, FAP, ITGBL1, MOXD1, COL10A1, and/or NALCN. In some embodiments, the fibrosis panel further comprises SCTR, EFEMPI, CLIC6, MMP7, THY1, and/or LOXL1. In some embodiments, the fibrosis gene panel further comprises MDFI, LTBP2, VTCN1, LUM, CLDN11, and/or CFAP221. In some embodiments, the fibrosis panel further comprises CFTR, DCDC2, EPCAM, ADRA2A, LAMC3, and/or AEBP1. In some embodiments, the fibrosis panel further comprises at least one of PAPLN, RASL11B, CDH6, PTGDS, LOXL4, and/or BHLHE22. In some embodiments, the fibrosis panel further comprises at least one of CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, and/or SNAP25. In some embodiments, at least one of the genes of the fibrosis panel are upregulated in connection with a disease. In some embodiments, at least one of the genes of the fibrosis panel are upregulated in NASH. In some embodiments the at least one gene of the fibrosis panel upregulated in NASH comprises at least one of STMN2, FAP, ITGBL1, MOXD1, COL10A1, NALCN, SCTR, EFEMPI, CLIC6, MMP7, THY1, LOXL1, MDFI, LTBP2, VTCN1, LUM, CLDN11, CFAP221, CFTR, DCDC2, EPCAM, ADRA2A, LAMC3, AEBP1, PAPLN, RASL11B, CDH6, PTGDS, LOXL4, BHLHE22, CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, SNAP25, GJA5, UBD, DTNA, LEF1, THBS2, PLCXD3, CTHRC1, SUSD2, SMOC2, SOD3, SPON1, PDZK1IP1, F3, MMP2, MFAP2, C7, CKMT2, CLDN10, CXCL6, AKR1B10, PHLDA3, COMP, CD40LG, PTK7, CCDC80, SEZ6L2, COL16A1, AQP1, GSN, GEM, NELL2, PAQR5, TYMS, LXN, TRAC, PLPP4, AP1M2, GPRC5B, VEPH1, RGS4, SPP1, OLR1, MGP, SLC7A6, BEX2, SIT1, ANO9, DPYSL3, CD1E, ANTXR1, BOC, LAYN, PDGFD, CCL21, HKDC1, CD5, CCL19, GPC3, OMG, FBLN2, TRAT1, SEMA3G, KRT7, ANKRD29, COL5A1, IGLC3, UBASH3A, PDGFA, NFASC, CCR6, LGR6, WFDC2, NPNT, VWF, MUC6, COL1A2, SLAMF1, IGHG1, CXCR3, F13A1, TACSTD2, SIRPG, STMN3, IGFBP7, CXCL1, CCR2, CCDC146, CTSK, COL1A1, HSPB2, RRAD, COL3A1, BACE2, ZMAT3, PCYOX1L, EEF1A2, CHI3L1, FXYD2, CCL20, CH25H, SEPTIN8, and/or CXCL8. In some embodiments, the genes of the fibrosis panel are downregulated in connection with a disease. In some embodiments, the genes of the fibrosis panel are downregulated in NASH. In some embodiments, the downregulated genes comprise at least one of DPPA4, EGFLAM, and/or MAT1A.

In some embodiments, the genes in the fibrosis panel comprise the first 48 genes listed above (which appear in 6 out of 6 lists), the first 66 genes listed above (which appear in 5+ out of 6 lists), and first 103 genes listed above (which appear in 4+ out of 6 lists). In some embodiments, the genes in the fibrosis panel comprise the first 48 genes listed above (which appear in 6 out of 6 lists). In some embodiments, the genes in the fibrosis panel comprise the first 66 genes listed above (which appear in 5+ out of 6 lists). In some embodiments, the genes in the fibrosis panel comprise the first 103 genes listed above (which appear in 4+ out of 6 lists).

In some embodiments, the at least one gene in the inflammation panel comprises at least one of LPL, STMN2, TREM2, FABP4, COMP, CAPG, SPP1, LOXL4, FABP5, THY1, EMILIN2, SLAMF8, BCAT1, CD300LB, CLDN11, DTNA, OLR1, MMP9, SPATA21, UBD, ITGBL1, CCL22, C15orf48, LGALS3, CXCL10, LTBP2, CPZ, KCNN4, COL1A1, DHRS9, LYZ, EFEMPI, THBS2, RTN1, CD24, IL32, HS3ST2, MOXD1, GPNMB, COL3A1, TTC9, CENPV, LOXL1, PDGFA, SCTR, COL1A2, CCL20, LAMC3, PAPLN, RAB7B, AEBP1, TP5313, MDFI, LUM, RGS10, CLIC6, RASLIOB, LAIR1, PLXNC1, ALOX5AP, PODN, LSP1, CD52, JAK3, VCAN, TNFRSF4, WNT4, KRT23, LRRC1, LAMA2, SNAP25, CD37, ITGAX, MTHFD2, CMYA5, DNAJC5B, PTPN7, DUSP8, PTGDS, MICAL1, MRAS, PRAMEF10, CCL17, DGKA, GEM, CD1E, CDH6, RRAD, MMP7, HAPLN3, GAPT, B3GNT5, CD5, NELL2, PBX4, SLC7A6, BHLHE22, FSTL3, CD48, CCR7, IL411, SLC1A7, SLC2A14, ADAM28, KLHL29, LAMA3, ACADSB, CCR5, GJA5, CD40LG, KRTCAP3, FCAMR, DNAJC12, SIT1, CYP2C19, EPHA3, RPS6KA1, FBLN2, WFDC2, CD6, CXCR3, VIL1, CD28, CHIT1, TRAF1, CD2, ANO9, SLC16A3, ABR, PDCD1, CD96, TRIM31, SUSD2, SAMD11, PLD4, CCR2, LRFN4, FGR, CACNB1, TMEM164, GRAP2, GABRE, MMP14, SIRPG, LGALS2, NCF2, CARMIL2, MACO1, SLCO1A2, MS4A14, PRAMEF33, TRPM2, LCK, EGFLAM, LILRB3, AQP8, GPR174, BATF, and/or MT1B. In some embodiments, the at least one gene comprises at least 10 of these genes. In some embodiments, the at least one gene comprises at least of these genes. In some embodiments, the at least one gene comprises at least 30 of these genes. In some embodiments, the at least one gene comprises at least 40 of these genes. In some embodiments, the at least one gene comprises at least 50 of these genes. In some embodiments, the at least one gene comprises at least 60 of these genes. In some embodiments, the at least one gene comprises at least 70 of these genes. In some embodiments, the at least one gene comprises at least 80 of these genes. In some embodiments, the at least one gene comprises at least 90 of these genes. In some embodiments, the at least one gene comprises at least 100 of these genes. In some embodiments, the at least one gene comprises at least 125 of these genes. In some embodiments, the at least one gene comprises at least 150 of these genes.

In some embodiments, the at least one gene in the inflammation panel comprises at least one of: i) LPL, STMN2, TREM2, FABP4, COMP, and/or CAPG. In some embodiments, the at least one gene comprises any two, any three, any four, any five, or all six of the genes.

In some embodiments, the at least one gene in the inflammation panel further comprises (in addition to i) above) at least one of: ii) SPP1, LOXL4, FABP5, THY1, EMILIN2, and/or SLAMF8. In some embodiments, the at least one gene comprises any two, any three, any four, any five, or all six of the genes.

In some embodiments, the at least one gene in the inflammation panel further comprises (in addition to i) and/or ii) above) at least one of: iii) BCAT1, CD300LB, CLDN11, DTNA, OLR1, and/or MMP9. In some embodiments, the at least one gene comprises any two, any three, any four, any five, or all six of the genes.

In some embodiments, the at least one gene in the inflammation panel further comprises (in addition to i) and/or ii) and/or iii) above) at least one of: iv) SPATA21, UBD, ITGBL1, CCL22, C15orf48, and/or LGALS3. In some embodiments, the at least one gene comprises any two, any three, any four, any five, or all six of the genes.

In some embodiments, the at least one gene in the inflammation panel further comprises (in addition to i) and/or ii) and/or iii) and/or iv) above) at least one of: v) CXCL10, LTBP2, CPZ, KCNN4, COL1A1, and/or DHRS9. In some embodiments, the at least one gene comprises any two, any three, any four, any five, or all six of the genes.

In some embodiments, the at least one gene in the inflammation panel further comprises (in addition to i) and/or ii) and/or iii) and/or iv) and/or v) above) at least one of: vi) LYZ, EFEMPI, THBS2, RTN1, CD24, IL32, HS3ST2, MOXD1, GPNMB, COL3A1, TTC9, CENPV, LOXL1, PDGFA, SCTR, COL1A2, CCL20, LAMC3, PAPLN, RAB7B, AEBP1, TP5313, MDFI, LUM, RGS10, CLIC6, RASLIOB, and/or LAIR1. In some embodiments, the at least one gene comprises any two, any three, any four, any five, any six, any seven, any eight, any nine, any ten, any eleven, any twelve, any thirteen, any fourteen, any fifteen, any sixteen, any seventeen, any eighteen, any nineteen, any twenty, any twenty-one, any twenty-two, any twenty-three, any twenty-four, any twenty-five, any twenty-six, any twenty-seven, or all twenty-eight of the genes.

In some embodiments, the at least one gene in the inflammation panel in the biological sample from the subject is upregulated compared to the corresponding gene in the reference population of subjects without liver inflammation. In some embodiments, the at least one gene in the inflammation panel in the biological sample from the subject that is upregulated compared to the corresponding gene in the reference population comprises at least one of LPL, STMN2, TREM2, FABP4, COMP, CAPG, SPP1, LOXL4, FABP5, THY1, EMILIN2, SLAMF8, BCAT1, CD300LB, CLDN11, DTNA, OLR1, MMP9, SPATA21, UBD, ITGBL1, CCL22, C15orf48, LGALS3, CXCL10, LTBP2, CPZ, KCNN4, COL1A1, DHRS9, LYZ, EFEMPI, THBS2, RTN1, CD24, IL32, HS3ST2, MOXD1, GPNMB, COL3A1, TTC9, LOXL1, PDGFA, SCTR, COL1A2, CCL20, LAMC3, PAPLN, RAB7B, AEBP1, TP5313, MDFI, LUM, RGS10, CLIC6, LAIR1, PLXNC1, ALOX5AP, PODN, LSP1, CD52, JAK3, VCAN, TNFRSF4, WNT4, KRT23, LRRC1, LAMA2, SNAP25, CD37, ITGAX, MTHFD2, DNAJC5B, PTPN7, DUSP8, PTGDS, MICAL1, MRAS, PRAMEF10, CCL17, DGKA, GEM, CD1E, CDH6, RRAD, MMP7, HAPLN3, GAPT, B3GNT5, CD5, NELL2, PBX4, SLC7A6, BHLHE22, FSTL3, CD48, CCR7, IL411, SLC1A7, SLC2A14, ADAM28, KLHL29, LAMA3, CCR5, GJA5, CD40LG, FCAMR, SIT1, EPHA3, RPS6KA1, FBLN2, WFDC2, CD6, CXCR3, CD28, CHIT1, TRAF1, CD2, ANO9, SLC16A3, ABR, PDCD1, CD96, TRIM31, SUSD2, SAMD11, PLD4, CCR2, LRFN4, FGR, CACNB1, TMEM164, GRAP2, GABRE, MMP14, SIRPG, LGALS2, NCF2, CARMIL2, MS4A14, PRAMEF33, TRPM2, LCK, LILRB3, AQP8, GPR174, and/or BATF. In some embodiments, at least 10 of these genes are upregulated compared to the corresponding genes in the reference population. In some embodiments, at least 20 of these genes are upregulated compared to the corresponding genes in the reference population. In some embodiments, at least 30 of these genes are upregulated compared to the corresponding genes in the reference population. In some embodiments, at least 40 of these genes are upregulated compared to the corresponding genes in the reference population. In some embodiments, at least 50 of these genes are upregulated compared to the corresponding genes in the reference population. In some embodiments, at least 60 of these genes are upregulated compared to the corresponding genes in the reference population. In some embodiments, at least 70 of these genes are upregulated compared to the corresponding genes in the reference population. In some embodiments, at least 80 of these genes are upregulated compared to the corresponding genes in the reference population. In some embodiments, at least 90 of these genes are upregulated compared to the corresponding genes in the reference population. In some embodiments, at least 100 of these genes are upregulated compared to the corresponding genes in the reference population. In some embodiments, at least 120 of these genes are upregulated compared to the corresponding genes in the reference population. In some embodiments, at least 140 of these genes are upregulated compared to the corresponding genes in the reference population.

In some embodiments, the at least one gene in the inflammation panel in the biological sample from the subject is downregulated compared to the corresponding gene in the reference population of subjects without liver inflammation. In some embodiments, the at least one gene in the biological sample from the subject that is down regulated compared to the corresponding gene in the reference population comprises at least one of CENPV, RASLIOB, CMYA5, ACADSB, KRTCAP3, DNAJC12, CYP2C19, VIL1, MACO1, SLCO1A2, EGFLAM, and/or MT1B. In some embodiments, at least any 2, at least any 3, at least any 4, at least any 5, at least any 6, at least any 7, at least any 8, at least any 9, at least any 10, at least any 11, or all 12 of these genes are downregulated compared to the corresponding genes in the reference population.

In some embodiments, the inflammation panel, which can be examined in regard to a biological sample from a subject, comprises at least one of LPL, STMN2, TREM2, FABP4, COMP, CAPG, SPP1, LOXL4, FABP5, THY1, EMILIN2, SLAMF8, BCAT1, CD300LB, CLDN11, DTNA, OLR1, MMP9, SPATA21, UBD, ITGBL1, CCL22, C15orf48, LGALS3, CXCL10, LTBP2, CPZ, KCNN4, COL1A1, DHRS9, LYZ, EFEMPI, THBS2, RTN1, CD24, IL32, HS3ST2, MOXD1, GPNMB, COL3A1, TTC9, CENPV, LOXL1, PDGFA, SCTR, COL1A2, CCL20, LAMC3, PAPLN, RAB7B, AEBP1, TP5313, MDFI, LUM, RGS10, CLIC6, RASLIOB, LAIR1, PLXNC1, ALOX5AP, PODN, LSP1, CD52, JAK3, VCAN, TNFRSF4, WNT4, KRT23, LRRC1, LAMA2, SNAP25, CD37, ITGAX, MTHFD2, CMYA5, DNAJC5B, PTPN7, DUSP8, PTGDS, MICAL1, MRAS, PRAMEF10, CCL17, DGKA, GEM, CD1E, CDH6, RRAD, MMP7, HAPLN3, GAPT, B3GNT5, CD5, NELL2, PBX4, SLC7A6, BHLHE22, FSTL3, CD48, CCR7, IL411, SLC1A7, SLC2A14, ADAM28, KLHL29, LAMA3, ACADSB, CCR5, GJA5, CD40LG, KRTCAP3, FCAMR, DNAJC12, SIT1, CYP2C19, EPHA3, RPS6KA1, FBLN2, WFDC2, CD6, CXCR3, VIL1, CD28, CHIT1, TRAF1, CD2, ANO9, SLC16A3, ABR, PDCD1, CD96, TRIM31, SUSD2, SAMD11, PLD4, CCR2, LRFN4, FGR, CACNB1, TMEM164, GRAP2, GABRE, MMP14, SIRPG, LGALS2, NCF2, CARMIL2, MACO1, SLCO1A2, MS4A14, PRAMEF33, TRPM2, LCK, EGFLAM, LILRB3, AQP8, GPR174, BATF, and/or MT1B. In some embodiments, at least one of the genes of the inflammation panel are upregulated in connection with a disease. In some embodiments, at least one of the genes hereinbefore set forth in this paragraph are upregulated in NASH except for at least one of CENPV, RASLIOB, CMYA5, ACADSB, KRTCAP3, DNAJC12, CYP2C19, VIL1, MACO1, SLCO1A2, EGFLAM, and/or MT1B. In some embodiments, at least one of the genes of the inflammation panel are downregulated in connection with a disease. In some embodiments, at least one of the genes of the inflammation panel are downregulated in NASH. In some embodiments, at least one of the genes of the inflammation panel downregulated in NASH comprises CENPV, RASLIOB, CMYA5, ACADSB, KRTCAP3, DNAJC12, CYP2C19, VIL1, MACO1, SLCO1A2, EGFLAM, and/or MT1B.

In some embodiments, the inflammation panel comprises at least one of LPL, STMN2, TREM2, FABP4, COMP, CAPG, SPP1, LOXL4, FABP5, THY1, EMILIN2, SLAMF8, BCAT1, CD300LB, CLDN11, DTNA, OLR1, MMP9, SPATA21, UBD, ITGBL1, CCL22, C15orf48, LGALS3, CXCL10, LTBP2, CPZ, KCNN4, COL1A1, DHRS9, LYZ, EFEMPI, THBS2, RTN1, CD24, IL32, HS3ST2, MOXD1, GPNMB, COL3A1, TTC9, CENPV, LOXL1, PDGFA, SCTR, COL1A2, CCL20, LAMC3, PAPLN, RAB7B, AEBP1, TP5313, MDFI, LUM, RGS10, CLIC6, RASLIOB, LAIR1, PLXNC1, ALOX5AP, PODN, LSP1, CD52, JAK3, VCAN, TNFRSF4, WNT4, KRT23, LRRC1, LAMA2, SNAP25, CD37, ITGAX, MTHFD2, CMYA5, DNAJC5B, PTPN7, DUSP8, PTGDS, MICAL1, MRAS, PRAMEF10, CCL17, DGKA, GEM, CD1E, CDH6, RRAD, MMP7, HAPLN3, GAPT, B3GNT5, CD5, NELL2, PBX4, SLC7A6, BHLHE22, FSTL3, CD48, CCR7, IL411, SLC1A7, SLC2A14, ADAM28, KLHL29, LAMA3, ACADSB, CCR5, GJA5, CD40LG, KRTCAP3, FCAMR, DNAJC12, SIT1, CYP2C19, EPHA3, RPS6KA1, FBLN2, WFDC2, CD6, CXCR3, VIL1, CD28, CHIT1, TRAF1, CD2, ANO9, SLC16A3, ABR, PDCD1, CD96, TRIM31, SUSD2, SAMD11, PLD4, CCR2, LRFN4, FGR, CACNB1, TMEM164, GRAP2, GABRE, MMP14, SIRPG, LGALS2, NCF2, CARMIL2, MACO1, SLCO1A2, MS4A14, PRAMEF33, TRPM2, LCK, EGFLAM, LILRB3, AQP8, GPR174, BATF, and/or MT1B. In some embodiments, at least one of the genes of the inflammation panel are upregulated in connection with a disease. In some embodiments, at least one of the genes hereinbefore set forth in this paragraph are upregulated in NASH except for at least one of CENPV, RASLIOB, CMYA5, ACADSB, KRTCAP3, DNAJC12, CYP2C19, VIL1, MACO1, SLCO1A2, EGFLAM, and/or MT1B. In some embodiments, at least one of the genes hereinbefore set forth in this paragraph upregulated in NASH comprise at least one of LPL, STMN2, TREM2, FABP4, COMP, CAPG, SPP1, LOXL4, FABP5, THY1, EMILIN2, SLAMF8, BCAT1, CD300LB, CLDN11, DTNA, OLR1, MMP9, SPATA21, UBD, ITGBL1, CCL22, C15orf48, LGALS3, CXCL10, LTBP2, CPZ, KCNN4, COL1A1, DHRS9, LYZ, EFEMPI, THBS2, RTN1, CD24, IL32, HS3ST2, MOXD1, GPNMB, COL3A1, TTC9, LOXL1, PDGFA, SCTR, COL1A2, CCL20, LAMC3, PAPLN, RAB7B, AEBP1, TP5313, MDFI, LUM, RGS10, CLIC6, LAIR1, PLXNC1, ALOX5AP, PODN, LSP1, CD52, JAK3, VCAN, TNFRSF4, WNT4, KRT23, LRRC1, LAMA2, SNAP25, CD37, ITGAX, MTHFD2, DNAJC5B, PTPN7, DUSP8, PTGDS, MICAL1, MRAS, PRAMEF10, CCL17, DGKA, GEM, CD1E, CDH6, RRAD, MMP7, HAPLN3, GAPT, B3GNT5, CD5, NELL2, PBX4, SLC7A6, BHLHE22, FSTL3, CD48, CCR7, IL411, SLC1A7, SLC2A14, ADAM28, KLHL29, LAMA3, CCR5, GJA5, CD40LG, FCAMR, SIT1, EPHA3, RPS6KA1, FBLN2, WFDC2, CD6, CXCR3, CD28, CHIT1, TRAF1, CD2, ANO9, SLC16A3, ABR, PDCD1, CD96, TRIM31, SUSD2, SAMD11, PLD4, CCR2, LRFN4, FGR, CACNB1, TMEM164, GRAP2, GABRE, MMP14, SIRPG, LGALS2, NCF2, CARMIL2, MS4A14, PRAMEF33, TRPM2, LCK, LILRB3, AQP8, GPR174, and/or BATF. In some embodiments, at least one of the genes of the inflammation panel are downregulated in connection with a disease. In some embodiments, at least one of the genes of the inflammation panel are downregulated in NASH. In some embodiments, at least one of the genes of the inflammation panel downregulated in NASH comprise at least one of CENPV, RASLIOB, CMYA5, ACADSB, KRTCAP3, DNAJC12, CYP2C19, VIL1, MACO1, SLCO1A2, EGFLAM, and/or MT1B.

In some embodiments, the inflammation panel comprises at least one of LPL, STMN2, TREM2, FABP4, COMP, and/or CAPG. In some embodiments, the inflammation panel further comprises at least one of SPP1, LOXL4, FABP5, THY1, EMILIN2, and/or SLAMF8. In some embodiments, the inflammation panel further comprises at least one of BCAT1, CD300LB, CLDN11, DTNA, OLR1, and/or MMP9. In some embodiments, the inflammation panel further comprises at least one of SPATA21, UBD, ITGBL1, CCL22, C15orf48, and/or LGALS3. In some embodiments, the inflammation panel further comprises at least one of CXCL10, LTBP2, CPZ, KCNN4, COL1A1, and/or DHRS9. In some embodiments, the inflammation panel further comprises at least one of LYZ, EFEMPI, THBS2, RTN1, CD24, IL32, HS3ST2, MOXD1, GPNMB, COL3A1, TTC9, CENPV, LOXL1, PDGFA, SCTR, COL1A2, CCL20, LAMC3, PAPLN, RAB7B, AEBP1, TP5313, MDFI, LUM, RGS10, CLIC6, RASL10B, and/or LAIR1.

In some embodiments, the genes in the inflammation panel comprise the first 15 genes listed above (which appear in 6 out of 6 lists), the first 36 genes listed above (which appear in 5+ out of 6 lists), the first 58 genes listed above (which appear in 4+ out of 6 lists), and the first 90 genes listed above (which appear in 3+ out of 6 lists). In some embodiments, the genes in the inflammation panel comprise the first 15 genes listed above (which appear in 6 out of 6 lists). In some embodiments, the genes in the inflammation panel comprise the first 36 genes listed above (which appear in 5+ out of 6 lists). In some embodiments, the genes in the inflammation panel comprise the first 58 genes listed above (which appear in 4+ out of 6 lists). In some embodiments, the genes in the inflammation panel comprise the first 90 genes listed above (which appear in 3+ out of 6 lists).

In any of the embodiments described herein, the biological sample comprises a sample from an organ, a tissue, a cell, and/or a biological fluid from the subject. In some embodiments, the organ comprises a liver. In some embodiments, the tissue comprises a connective tissue, a muscle tissue, a nervous tissue, an epithelial tissue, a parenchyma, or a lobule. In some embodiments, the cell comprises a hepatocyte, a Kupffer cell, a nonparenchymal cell, a sinusoidal endothelial cell, or a hepatic stellate cell, an intrahepatic lymphocyte, a liver-specific natural killer cell, an αβ T cell, or a γδ T cell. In some embodiments, the biological fluid comprises plasma, serum, lymph, semen, and/or a mucosal secretion. In some embodiments, the biological sample comprises blood, semen, saliva, urine, feces, hair, teeth, bone, tissue, or a buccal sample. In some embodiments, the biological sample is obtained from the subject by a biopsy. In some embodiments, the biological sample is a liver biopsy.

In some embodiments, RNA expression can be determined in part by RNA sequencing. In some embodiments, RNA sequencing reads can be mapped to a genome. In some embodiments, the genome is the human genome. In some embodiments, the human genome is reference assembly GRCh38. In some embodiments, the RNA sequencing reads can be limited to those for at least one protein coding gene, at least one long non-coding RNA, at least one mitochondrial rRNA, at least one mitochondrial tRNA, at least one rRNA, at least one ribozyme, at least one B-cell receptor subunit constant gene, and/or at least one T-cell receptor subunit constant gene. In some embodiments, the RNA sequencing reads are not so limited. In some embodiments, the sequences can be mapped without strand specificity, with strand-specific reverse first-read mapping, or with strand-specific forward first-read mapping. In some embodiments, the sequences can be mapped using kallisto v0.45.0 with strand-specific reverse first-read mapping (Bray et al., Nat. Biotechnol., 2016, 34, 525). In some embodiments, transcript counts can be aggregated to gene counts. In some embodiments, the aggregation can be conducted using tximport (Soneson et al., F1000Research, 2015, 4, 1521).

In some embodiments, the selection of genes for inclusion in a gene panel can be first filtered based on one or more criteria. In some embodiments, the selection of genes for inclusion in a gene panel can be filtered based on their biotype. In some embodiments, the biotypes can be limited to protein coding genes, long non-coding RNAs, mitochondrial rRNAs, mitochondrial tRNAs, rRNAs, ribozymes, B-cell receptor subunit constant genes, and/or T-cell receptor subunit constant genes. In some embodiments, the biotypes can be limited to protein coding genes, B-cell receptor subunit constant genes, and/or T-cell receptor subunit constant genes. In some embodiments, the selection of genes for inclusion in a gene panel can be filtered based on their location in the human genome. In some embodiments, the human genome is reference assembly GRCh38. In some embodiments, genes can be limited to genes placed and localized on chromosomes 1 to 22 and/or the X chromosome.

In some embodiments, median or mean transcript per million (TPM) values for each gene can be determined, either across the entire dataset or within each disease category. In some embodiments, genes having a median TPM value greater than a predetermined value are considered genes of interest. In some embodiments, the predetermined median TPM value is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0. In some embodiments, the predetermined median TPM value is 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0. In some embodiments, the predetermined median TPM value is 0.5. In some embodiments, the genes of interest are limited to those having at least a median TPM value of 0.5 in at least 1, 2, 3, or more to the total number of datasets graded for fibrosis F3 or higher (F3+), on the one hand, or F0 or F1 (F0/F1) on the other. In some embodiments, genes of interest are limited to those having at least a median TPM value of 0.5 in at least 1, 2, 3, or more to the total number of datasets graded for inflammation 0 or 2 and higher (2+). In some embodiments, genes with detectable RNA expression counts or counts greater than 1, 2, 3, 10, 50, 100 in at least 1, 2, 3, 4, 5, 10, or 20 subjects are considered genes of interest.

In some embodiments, metrics for each gene in the comparison of fibrosis F3+ versus F0/F1 can be calculated. In some embodiments, metrics for each gene in the comparison of fibrosis F3+ versus F0 can be calculated. In some embodiments, metrics for each gene in the comparison of fibrosis F2 or higher (F2+) versus F0/F1 can be calculated. In some embodiments, metrics for each gene in the comparison of fibrosis F2+ versus F0 can be calculated. In some embodiments, metrics for each gene in the comparison of fibrosis F1 or higher (F1+) versus F0 can be calculated. In some embodiments, metrics for each gene in the comparison of inflammation 2+ versus 0 can be calculated. In some embodiments, metrics for each gene in the comparison of inflammation 2+ versus inflammation 0 or 1 (0/1) can be calculated. In some embodiments, metrics for each gene in the comparison of inflammation 1 and higher (1+) versus 0 can be calculated. In some embodiments, the metric is a difference value such as fold change or absolute difference, a statistical significance value, and/or a classification metric, such as accuracy, false positive rate, true positive rate (recall), true negative rate (specificity), positive predictive value (precision), negative predictive value, precision-recall F measure (F1 score), area-under-the-receiver-operating-characteristic-curve (ROCAUC), or area-under-the-precision-recall-curve value (PRAUC). In some embodiments, differential gene expression analysis can be performed to obtain measures of fold change and statistical significance of fold change. In some embodiments, the statistical significance of fold change can be adjusted for multiple comparisons. In some embodiments, the Benjamini-Hochberg method is used to adjust for multiple comparisons. In some embodiments, DESeq2, EdgeR, limma, or voom can be used to perform the differential gene expression analysis. In some embodiments, DESeq2 can be used to perform the differential gene expression analysis with sex, age, genotype, disease status, and/or drug or medication use as a covariate (Love et al., Genome Biol., 2014, 15, 550, pp 2084-2092). In some embodiments, fold changes estimates obtained from differential gene expression analysis can be shrunken toward zero, as recommended by Love et al. for ranking genes. In some embodiments, the adaptive shrinkage estimator from the ashr package (Stephens, Biostatistics, 2017, 18, 2, 275-294), the normal shrinkage estimator in DESeq2, or the adaptive t prior shrinkage estimator from the apeglm package (Zhu et al., Bioinformatics, 2019, 35, 12, 2084-2092) can be used. In some embodiments where there is a dataset imbalance of negative (F0/F1 for fibrosis or 0 for inflammation) and positive (F3+ for fibrosis or 2+ for inflammation) samples, PRAUC values can be used to quantify the ability of each gene's RNA expression level to correctly classify subjects into disease stages (fibrosis F3+ versus F0/F1 or inflammation 2+ versus 0) (Saito and Rehmsmeier, PLoS ONE, 2015, 10, e0118432). In some embodiments where there is a dataset imbalance of negative (F0/F1 for fibrosis or 0 for inflammation) and positive (F3 for fibrosis or 2+ for inflammation) samples, a balanced dataset can be obtained with stratified sampling with replacement, and ROCAUC values can be used to quantify the ability of each gene's RNA expression level to correctly classify subjects into disease stages (fibrosis F3+ versus F0/F1 or inflammation 2+ versus 0). In some embodiments where there is no such database imbalance, ROCAUC values can be used to quantify the ability of each gene's RNA expression level to correctly classify subjects into disease stages (fibrosis F3+ versus F0/F1 or inflammation 2+ versus 0). In some embodiments, PRAUC values can be calculated on gene count values or on TPM values. In some embodiments, the gene count values can be adjusted for RNA composition bias. In some embodiments, the adjustment is a size factor correction in DESeq2 or a trimmed mean of M values (TMM) factor correction.

In some embodiments, genes for a dataset can be ranked in descending order by, for example, fold change. In some embodiments, genes for a dataset can be ranked in ascending order by p-value level of statistical significance of fold change. In some embodiments, genes for a dataset can be ranked in descending order by PRAUC value. In some embodiments, genes for a dataset can be ranked in descending order by ROCAUC value. In some embodiments, genes for a dataset can be ranked in descending order by F1 score. In some embodiments, genes for a dataset can be ranked in descending order by accuracy. In some embodiments, the top 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 125, 130, 140, 150, 160, 170, 175, 200, 225, 250, 275, 300, 350, 400, 450, 500, 600, 650, 700, 750, 800, 850, 900, 950, 1000, 1100, 1200, 1300, 1400, 1500, 1600, 1700, 1800, 1900, 2000, 3000, 3500, 4000, 4500, 5000, 5500, 6000, 6500, 7000, 7500, 8000, 8500, 9000, 9500, or 10000 ranked genes are selected from each list. In some embodiments, the number of times that each gene in the top 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 125, 130, 140, 150, 160, 170, 175, 200, 225, 250, 275, 300, 350, 400, 450, 500, 600, 650, 700, 750, 800, 850, 900, 950, 1000, 1100, 1200, 1300, 1400, 1500, 1600, 1700, 1800, 1900, 2000, 3000, 3500, 4000, 4500, 5000, 5500, 6000, 6500, 7000, 7500, 8000, 8500, 9000, 9500, or 10000 ranked genes appears in the top 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 125, 130, 140, 150, 160, 170, 175, 200, 225, 250, 275, 300, 350, 400, 450, 500, 600, 650, 700, 750, 800, 850, 900, 950, 1000, 1100, 1200, 1300, 1400, 1500, 1600, 1700, 1800, 1900, 2000, 3000, 3500, 4000, 4500, 5000, 5500, 6000, 6500, 7000, 7500, 8000, 8500, 9000, 9500, or 10000 ranked genes for the at least 1, 2, 3, 4, 5, 6, 7, 8, 9, 10, 11, 12, 15, 50, 100, or 1000 datasets is determined. In some embodiments, the number of times that each gene is statistically significant after multiple comparison adjustment for fold change in 1, 2, 3, 4, 5, 6, 7, 8, 9, 10, 11, 12, 15, 50, 100, or 1000 datasets is determined. In some embodiments, genes appearing in the top 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 125, 130, 140, 150, 160, 170, 175, 200, 225, 250, 275, 300, 350, 400, 450, 500, 600, 650, 700, 750, 800, 850, 900, 950, 1000, 1100, 1200, 1300, 1400, 1500, 1600, 1700, 1800, 1900, 2000, 3000, 3500, 4000, 4500, 5000, 5500, 6000, 6500, 7000, 7500, 8000, 8500, 9000, 9500, or 10000 ranked genes for the at least 1, 2, 3, 4, 5, 6, 7, 8, 9, 10, 11, 12, 15, 50, 100, or 1000 datasets and statistically significant after multiple comparison adjustment for fold change in 1, 2, 3, 4, 5, 6, 7, 8, 9, 10, 11, 12, 15, 50, 100, or 1000 datasets are selected to form a fibrosis panel. In some embodiments, genes appearing in the top 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 125, 130, 140, 150, 160, 170, 175, 200, 225, 250, 275, 300, 350, 400, 450, 500, 600, 650, 700, 750, 800, 850, 900, 950, 1000, 1100, 1200, 1300, 1400, 1500, 1600, 1700, 1800, 1900, 2000, 3000, 3500, 4000, 4500, 5000, 5500, 6000, 6500, 7000, 7500, 8000, 8500, 9000, 9500, or 10000 ranked genes for the at least 1, 2, 3, 4, 5, 6, 7, 8, 9, 10, 11, 12, 15, 50, 100, or 1000 datasets and statistically significant after multiple comparison adjustment for fold change in 1, 2, 3, 4, 5, 6, 7, 8, 9, 10, 11, 12, 15, 50, 100, or 1000 datasets are selected to form an inflammation panel.

In some embodiments, where smaller subsets of the gene panel are used, genes in the panel can be sorted in descending order first by the number of top lists each appeared in and second by mean or median rank across all the lists used to create the panel. In some embodiments, where smaller subsets of the gene panel are used, genes in the panel can be sorted by mean or median rank across all the lists used to create the panel. In some embodiments, where smaller subsets of the gene panel are used, genes in the panel can be sorted by mean or median rank across fold change lists used to create the panel. In some embodiments, where smaller subsets of the gene panel are used, genes in the panel can be sorted by mean or median rank across PRAUC lists used to create the panel.

In some embodiments, the determination of a subject's TS comprises determining the RNA expression level(s) of one or more genes in a biological sample from a subject, comparing this RNA expression with the RNA expression of a corresponding gene from a reference population of subjects without liver inflammation and/or without liver fibrosis, determining the relative difference in RNA expression, and integrating the changes in the individual RNA expression into an aggregate TS. In some embodiments, the determination of a subject's TS comprises determining the RNA expression level(s) of one or more genes in multiple biological samples from a subject, determining the relative difference in RNA expression across the multiple samples, and integrating the changes in the individual RNA expression into an aggregate TS. In some embodiments, the genes whose RNA expression level(s)s are measured include protein-coding genes, long non-coding RNAs, mitochondrial rRNAs, mitochondrial tRNAs, rRNAs, ribozymes, B-cell receptor subunit constant genes, and/or a T-cell receptor subunit constant genes. In some embodiments, the relative difference in RNA expression of genes in the panel are compared to the relative difference in RNA expression of genes not in the panel.

In some embodiments, a gene set enrichment analysis (GSEA) can be performed to derive a transcriptomic score for a sample using any of the gene panels disclosed herein.

In some embodiments, the GSEA can be performed as described in Subramanian et al., Proc. Natl. Acad. Sci. USA, 2005, 102, 15545. In some embodiments, the GSEA can be performed as described in Subramanian but modified and calculated using a custom R code.

In some embodiments, the GSEA can be performed as described in Lim et al., Pacific Symposium on Biocomputing, 2009, 14, 504-515, referred to herein as “GSEA2”. In some embodiments, the GSEA2 can be performed as described in Lim but modified and calculated using a custom R code.

In some embodiments, a single-sample GSEA or GSEA2 can be performed with standardized values using z-scores. In some embodiments, genes that are detected in less than 1%, 5%, 10%, 15%, 20%, 25%, 30%, 40%, 50%, 60%, 70%, 80%, 90%, or 95% of samples in the dataset are filtered out prior to GSEA or GSEA2. In some embodiments, z-scores can be calculated for each sample for each gene using “trimmed” means and standard deviations for that gene across the dataset with the largest 1%, 2%, or 5% and smallest 1%, 2%, or 5% of values trimmed from calculation of the mean and standard deviation. In some embodiments, modified z-scores can be calculated for each sample for each gene using the median and median absolute deviation of the dataset for that gene. In some embodiments, z-scores can be calculated for each gene in a total universe of size N, where N is the total number of genes whose RNA expression is detected in at least 1%, 5%, 10%, 15%, 20%, 25%, 30%, 40%, 50%, 60%, 70%, 80%, 90%, or 95% of the samples in the dataset.

In some embodiments, for GSEA, for each sample, the genes can be rank ordered by their z-score. In some embodiments, a gene panel S with size N_{H} can be divided into the subset of genes that trend up or down with disease stage based on their computed fold changes, named Sᵤₚ and S_{down} with sizes N_{H,up} and N_{H,down}, respectively. For each S, NH in Sᵤₚ, N_{H,up}, S_{down}, and N_{H,down}:

$${P}_{hit} ( S , i ) = {\sum }_{{g}_{i} ∈ S , j \leq i} \frac{min ( \vert {z}_{j} \vert , 3 )}{{N}_{R}} , where {N}_{R} = {\sum }_{{g}_{i} ∈ S} min ( \vert {z}_{j} \vert , 3 ) \\ {P}_{miss} ( S , i ) = {\sum }_{{g}_{i} ∉ S , j \leq i} \frac{1}{( N - {N}_{H} )}$$

In some embodiments, an enrichment score ES(S) can be calculated for each S:

$$For {S}_{up} : {max}_{1 \leq i \leq N} ( {P}_{hit} ( S , i ) - {P}_{miss} ( S , i ) , 0 ) \\ For {S}_{down} : {min}_{1 \leq i \leq N} ( {P}_{hit} ( S , i ) - {P}_{miss} ( S , i ) , 0 )$$

In some embodiments, a normalized enrichment score

$$NES ( S ) = \frac{ES ( S )}{\frac{1}{k} {\sum }_{1}^{k} ES ( {S}_{r} )} ,$$

where Sᵣ is one of k random gene sets with the same set size as S, is calculated. In some embodiments, the mean or median enrichment score of k random gene sets of the same set size as S can be used as normalization. In some embodiments, k is 10, 20, 50, 100, 200, 500, 1000, 2000, 5000, or 10000. In some embodiments, the NES for the gene panel S is calculated as the weighted average of the NES calculated with the subset of genes that trend up with disease, NES(Sᵤₚ), and of the NES calculated with the subset of genes that trend down with disease, NES(S_{down}). Namely,

$$NES ( S ) = \frac{{N}_{H , up} \cdot NES ( {S}_{up} ) + {N}_{H , down} \cdot NES ( {S}_{down} )}{{N}_{H}} .$$

In some embodiments, the NES(S) score is the transcriptomic score.

In some embodiments, for GSEA2, within each sample, both the z-scores and the additive inverse of the z-scores of the genes can be combined into a list of size 2 N, with directional labels for each gene of “up” (for z-scores) or “down” (for the additive inverse of z-scores), and then rank ordered by value. Each gene in the gene panel S with size N_{H} can be labeled as trending “up” or “down” with disease stage based on their computed fold changes, resulting in directional gene panel S′. In some embodiments, for each directional gene panel S′, where g′ denotes a gene with a directional label,

$${P}_{hit} ( {S}^{′} , i ) = {\sum }_{{g}_{i}^{′} ∈ {S}^{′} , j \leq i} \frac{min ( \vert {z}_{j} \vert , 3 )}{{N}_{R}} , \\ where {N}_{R} = {\sum }_{{g}_{i}^{′} ∈ {S}^{′}} min ( \vert {z}_{j} \vert , 3 ) \\ {P}_{miss} ( {S}^{′} , i ) = {\sum }_{{g}_{i}^{′} ∉ {S}^{′} , j \leq i} \frac{1}{( 2 N - {N}_{H} )}$$

For each directional gene panel S′, an enrichment score ES(S′) can be calculated:

$${max}_{1 \leq i \leq 2 N} ( {P}_{hit} ( {S}^{′} , i ) - {P}_{miss} ( {S}^{′} , i ) , 0 )$$

For each S′, a normalized enrichment score

$$NES ( {S}^{′} ) = \frac{ES ( {S}^{′} )}{\frac{1}{k} {\sum }_{1}^{k} ES ( {S}_{r}^{′} )} ,$$

where S′ is one of k random directional gene sets with size N_{H}, can be calculated. In some embodiments, the mean or median enrichment score of k random gene sets of the same set size can be used as normalization. In some embodiments, k is 10, 20, 50, 100, 200, 500, 1000, 2000, 5000, or 10000. In some embodiments, the NES(S′) score is the transcriptomic score.

In some embodiments, a gene set enrichment analysis with multiple-sample GSEA or GSEA2 can be performed to derive a TS for multiple longitudinal samples from a subject using any of the gene panels disclosed herein. In some embodiments, multiple-sample GSEA or GSEA2 can be performed with fold change of each gene between the multiple samples, in lieu of standardized z-scores.

In some embodiments, TSs from subsets of the gene panel can be determined with the same GSEA or GSEA2 methodology.

In some embodiments, subjects having a transcriptomic score greater than a threshold transcriptomic score determined from a reference population of subjects without liver inflammation and/or without liver fibrosis indicates that subject have liver inflammation and/or liver fibrosis. Subjects having a transcriptomic score that is equal to or less than a threshold transcriptomic score determined from a reference population of subjects without liver inflammation and/or without liver fibrosis indicates that subject does not have liver inflammation and/or liver fibrosis. As stated hereinbefore, the magnitude of any particular TS can also help characterize the degree of liver inflammation and/or liver fibrosis.

In addition, as stated hereinbefore, the TS can comprise a value determined from changes in RNA expression of genes of longitudinal liver samples from the subject. In some embodiments, TS comprises a value determined from changes in RNA expression across multiple biological samples from the subject. In some embodiments, subjects having a later transcriptomic score greater than an earlier transcriptomic score have progression of liver inflammation and/or liver fibrosis. In some embodiments, subjects having a later transcriptomic score that is less than an earlier transcriptomic score have regression of liver inflammation and/or liver fibrosis.

In some embodiments, the classification performance of the computed transcriptomic score using smaller subsets of the gene panel remains consistent with the full set.

In some embodiments, the classification performance of the computed transcriptomic score can be evaluated using metrics. In some embodiments, the metric computed is accuracy, false positive rate, true positive rate (recall), true negative rate (specificity), positive predictive value (precision), negative predictive value, precision-recall F measure (F1 score), area-under-the-receiver-operating-characteristic-curve (ROCAUC), or area-under-the-recprecision-recall-curve value (PRAUC). In some embodiments, the classified classes are F3+ vs F0/F1 for a fibrosis gene panel and 2+ vs 0 for an inflammation gene panel.

In some embodiments, the performance of the gene panel trained on the training dataset and tested on the holdout testing dataset can be comparable to that achieved with the gene panel trained and tested on the entire dataset.

In some embodiments, single-cell RNA-seq can be used to validate the selection and placement of genes in the fibrosis panel or the inflammation panel. In some embodiments, single cells from liver biopsies can be clustered into cell types following the gene signatures in Ramachandran et al., Nature, 2019, 575, 512-518. In some embodiments, the liver biopsies are from subjects with cirrhosis. In some embodiments, the liver biopsies are from subjects with NASH and/or NAFLD. In some embodiments, the liver biopsies are from non-diseased subjects. In some embodiments, single cells can be labeled as cholangiocyte cells, mesenchymal cells, endothelial cells, B cells, innate lymphoid cells, mononuclear phagocytes, plasmacytoid dendritic cells, T cells, plasma cells, or hepatocyte cells. In some embodiments, single cells labeled as mesenchymal cells can be re-clustered into more specific cell subtypes to identify hepatic stellate cells and scar-associated mesenchymal cells. In some embodiments, single cells labeled as mononuclear phagocytes can be similarly re-clustered to identify scar-associated macrophages. In some embodiments, genes in the gene panel with detected expression in any immune or fibrotic cell types can be identified. In some embodiments, the percent expression across cell types can be plotted on a heatmap. In some embodiments, genes can be hieratically clustered and their placement in either fibrosis and/or inflammation panels labeled, revealing that gene expression in immune and fibrotic cell types from external single-cell RNA-seq largely align with gene panels. Single cell analysis and clustering can be done with various computational packages, including python package SCANPY (Wolf at al., Genome Biol, 2018, 19, 15) and/or R package Seurat (Hao et al., Cell, 2021, 184, 3573-3587).

In some embodiments, where validation of the gene panel selection methodology is sought, performance of a gene panel selected using a subset of the cohorts can be tested on a holdout cohort. In some embodiments, fold changes and classification metrics can be calculated for each cohort except for the holdout cohort. In some embodiments, genes appearing in at least 1, 2, 3, 4, 5, 6, 7, 8, 9, 10, 11, 12, 15, 50, 100, or 1000 of the top 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 125, 130, 140, 150, 160, 170, 175, 200, 225, 250, 275, 300, 350, 400, 450, 500, 600, 650, 700, 750, 800, 850, 900, 950, 1000, 1100, 1200, 1300, 1400, 1500, 1600, 1700, 1800, 1900, 2000, 3000, 3500, 4000, 4500, 5000, 5500, 6000, 6500, 7000, 7500, 8000, 8500, 9000, 9500, or 10000 gene lists can be selected as the gene panel.

In some embodiments, where validation of the gene panel selection methodology is sought, performance of a gene panel selected using a training data subset can be tested on a holdout testing data subset In some embodiments, each cohort can be split into a s % training dataset and a (100−s) % testing dataset, where s is an integer between 1 and 99, within the strata of each fibrosis grade (F3+ vs. F0/F1). In some embodiments, each cohort can be split into a s % training dataset and a (100−s) % testing dataset, where s is an integer between 1 and 99, within the strata of each inflammation grade (2+ vs. 0). In some embodiments, fold changes and classification metrics can be calculated for each cohort using only the training dataset. In some embodiments, genes appearing in at least 1, 2, 3, 4, 5, 6, 7, 8, 9, 10, 11, 12, 15, 50, 100, or 1000 of the top 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 125, 130, 140, 150, 160, 170, 175, 200, 225, 250, 275, 300, 350, 400, 450, 500, 600, 650, 700, 750, 800, 850, 900, 950, 1000, 1100, 1200, 1300, 1400, 1500, 1600, 1700, 1800, 1900, 2000, 3000, 3500, 4000, 4500, 5000, 5500, 6000, 6500, 7000, 7500, 8000, 8500, 9000, 9500, or 10000 gene lists can be selected as the gene panel.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for.

### EXAMPLES

### Example 1: Derivation of Gene Panels

Three datasets (cohorts) of RNA samples paired with histopathological data contributed to the analysis exemplified herein. Bulk RNA was extracted from liver biopsies obtained from 2,528 patients undergoing bariatric surgery from 2005-17 in the Geisinger Health System MyCode Community Health Initiative cohort, of which 993 samples had been sequenced at Geisinger (GHS) and 1,535 at Regeneron (REGN). A publicly available dataset with 216 bulk RNA-sequencing of liver biopsies from control, NAFLD, and NASH patients was used as a third dataset (Govaere et al., Sci. Transl. Med., 2019, 12, eaba4448 (Govaere)). From the GHS and REGN cohorts, only samples with paired histopathology showing steatosis and with fibrosis and lobular inflammation histopathology values were used in the selection of gene panels, resulting in 726 and 1,214 samples, respectively. From the Govaere cohort, only samples with paired histopathology showing steatosis and with fibrosis histopathology values were used in the selection of gene panels, resulting in 206 samples.

Fibrosis and lobular inflammation (the latter referred to in this example as inflammation) on histopathology of the samples were scored following the NASH Clinical Research Network system. Specifically, fibrosis was scored as: no fibrosis, 0; portal fibrosis without septa, 1; portal fibrosis with septa, 2; bridging fibrosis, 3; between briding fibrosis and cirrhosis, 3-4; and cirrhosis, 4, while inflammation was scored as: no foci, 0; <2 foci/200×, 1; 2-4 foci/200×, 2; and >4 foci/200×, 3.

RNA sequencing reads were mapped to individual transcripts in the human genome reference assembly GRCh38 limited to the following features (see, world wide web at “useast.ensembl.org/info/genome/genebuild/biotypes.html”): protein coding genes, long non-coding RNAs, mitochondrial rRNAs, mitochondrial tRNAs, rRNAs, ribozymes, B-cell receptor subunit constant genes, and T-cell receptor subunit constant genes. Sequences were mapped using kallisto v0.45.0 with strand-specific reverse first-read mapping (Bray et al., Nat. Biotechnol., 2016, 34, 525). Transcript counts were aggregated to gene counts using tximport (Soneson et al., F1000Research, 2015, 4, 1521).

Two gene panels were created, one for classifying fibrosis and one for inflammation. Selection and ranking of genes in the fibrosis gene panel used data from the GHS, REGN, and Govaere cohorts. Selection of genes in the inflammation panel used data from the GHS and REGN cohorts.

Named sample-level inflammation histopathology values were not publicly available for the samples in Govaere et al. However, named sample-level values for fibrosis, NAFLD Activity Score (NAS), and disease group (NAFL or NASH) were publically available, and a heatmap linking unnamed sample-level information between fibrosis, NAS, disease group, and inflammation values was available. Inflammation values of 2 and 3 were grouped into a single category of 2+. Named samples sharing the same combinations of fibrosis, NAS, and disease group could be matched to inflammation values sharing the same three parameters. In some cases, the match was one-to-one, such that a combination of fibrosis, NAS, and disease group uniquely identified an inflammation value. In other cases, the match was a supermajority, such that more than 70% of samples sharing the same combination of fibrosis, NAS, and disease group could be linked to an inflammation value over other possibilities. Both of these cases were used to obtain samples that likely had an inflammation value of 0 or of 2+. 11 samples were identified to have an inflammation value of 0, and 64 samples were identified to likely have an inflammation value of 2+ (of which 6 samples had an inflammation value of 1, and 58 samples had an inflammation value of 2+), constituting samples from the Govaere cohort that were used for this analysis. Ranking of genes in the inflammation panel used data from the GHS, REGN, and Govaere (as inferred) cohorts.

For the selection of both the fibrosis and inflammation gene panels, genes were first filtered using two criteria: i) that the genes were protein coding genes, B-cell receptor subunit constant genes, or T-cell receptor subunit constant genes, and ii) that the genes were placed and localized on chromosomes 1 to 22 or the X chromosome, according to the human genome reference assembly GRCh38.

Within each cohort and within each disease stage, median transcript-per-million (TPM) values across samples were determined for each gene passing the previous filtering step. Genes having a median TPM value of at least 0.5 in a disease stage were subsequently considered genes of interest. For the selection of the fibrosis gene panel, genes of interest were limited to those having at least a median TPM value of 0.5 in fibrosis F3 or higher (F3+) or F0 or F1 (F0/F1) in at least two of the GHS, REGN, and Govaere cohorts. For the selection of the inflammation gene panel, genes of interest were limited to those having at least a median TPM value of 0.5 in at least one disease stage of inflammation 2 or 3 (2+) or 0 in the GHS and REGN cohorts.

For each cohort and for each gene of interest, two metrics were calculated for each gene's RNA expression level in the comparison of fibrosis F3+ versus F0/F1 or inflammation 2+ versus 0. These metrics were fold-change and precision-recall area-under-the-curve (PRAUC). To calculate fold change, differential gene expression analysis was performed using DESeq2 with sex as a covariate (Love et al., Genome Biol., 2014, 15, 550). Fold changes estimates were shrunken toward zero, as recommended by Love et al. for ranking genes. In particular, the adaptive shrinkage estimator from the ashr package was used (Stephens, Biostatistics, 2017, 18, 2, 275-294). Because the GHS and REGN datasets were imbalanced with many more F0/F1 (negatives) than F3 (positive) samples, PRAUC, instead of a receiver-operator-curve area-under-the-curve (ROCAUC), was used to quantify each gene's classification ability between the two categories (for the fibrosis panel, F3+ versus F0/F1, or for the inflammation panel, 2+ versus 0) (Saito and Rehmsmeier, PloS ONE, 2015, 10, e0118432). An ROCAUC compares the true positive rate (also known as sensitivity and recall) with the false positive rate. In contrast, a PRAUC compares true positive rate with precision (also known as positive predictive value). For calculating the PRAUC, neither true positive rate nor precision depends on the number of true negatives in the dataset. Thus, the PRAUC better captures the classification ability of genes in an imbalanced dataset with many negative cases. PRAUC values were calculated on gene count values, adjusted for RNA composition bias with size factor correction in DESeq2. In an alternative approach, a balanced dataset can be obtained with stratified sampling with replacement, and a ROCAUC calculated from such balanced dataset.

For each of the GHS, REGN, and Govaere cohorts, genes were ranked in descending order by maximum absolute value of log 2 fold change or PRAUC value. The top 200 genes from each list of the six lists (one of fold change and one of PRAUC value from each cohort) were obtained. For the fibrosis panel, the number of times each gene appeared across the six top-200 lists was counted. Genes that appeared in at least 3 of the 6 top-200 gene lists and were statistically significant for fold change at α=0.05 after Benjamini-Hochberg multiple comparison adjustment in at least 2 cohorts were included in the final fibrosis panel. For the inflammation panel, the Govaere cohort was not used for gene selection but for ranking genes in the selected gene panel. For the inflammation panel, the number of times each gene appeared across the four top-200 lists from the GHS and REGN cohorts was counted. Genes that appeared in at least 2 of the 4 top 200 gene lists and were statistically significant for fold change at α=0.05 after Benjamini-Hochberg multiple comparison adjustment in both GHS and REGN cohorts were included in the final inflammation panel.

Genes in the fibrosis gene panels were sorted in descending order first by the number of top-200 lists each appeared in (out of six lists from GHS, REGN, and Govaere) and second by median rank across the six lists. Genes in the inflammation gene panels were sorted in descending order first by the number of top-200 lists each appeared in (out of six lists from GHS, REGN, and Govaere) and second by median rank across the six lists.

### Flow Diagram of Selection of Gene Panel

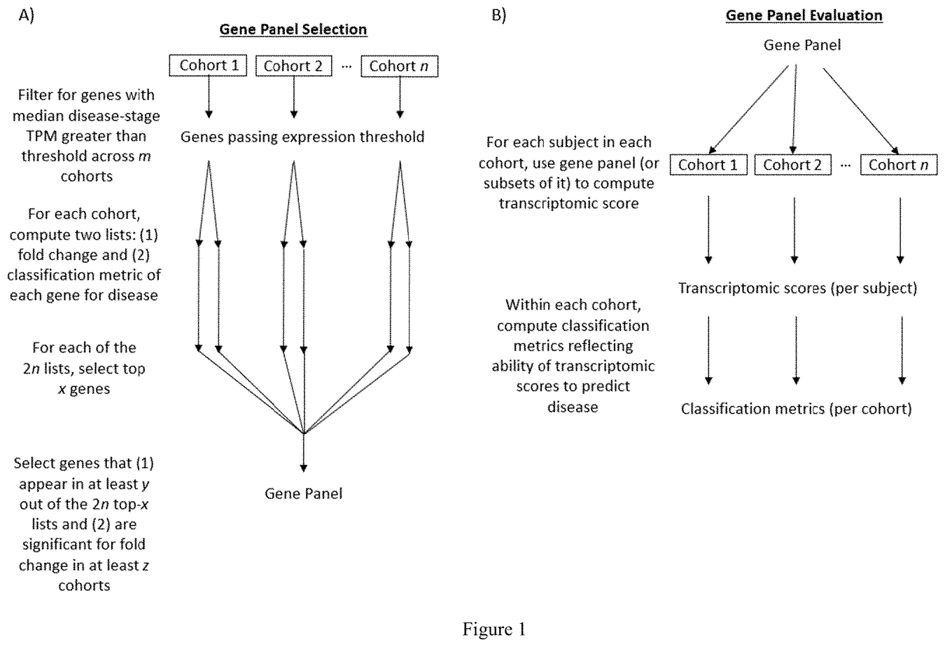

FIG. 1 (Panel A) shows a flow diagram of the gene panel selection. Genes with median disease-stage TPMs greater than a threshold TPM in at least m cohorts are selected. For each of the n number of cohorts, magnitudes of fold change and classification metrics (such as area under the curve or significance of fold change) of each gene for the desired disease comparison are computed. This results in 2n lists. Genes in each list are ranked by their value (e.g. descending order for magnitude of fold change, descending for area under the curve, ascending for p-value significance). The top-x genes from each list are selected. Genes that (1) appear in at least y out of the total 2n top-x lists and (2) are significant for fold change in at least z cohorts are selected for the gene panel. Genes in the gene panel are first ranked by the number of top-x lists they appear in and, within each category, by the median rank across the lists. In this example, for the fibrosis panel and 70% split fibrosis panel, n=3, m=2, threshold TPM=0.5, x=200, y=3, and z=2; for the inflammation panel, 70% split inflammation panel, GHS-REGN fibrosis panel, GHS-Govaere fibrosis panel, and REGN-Govaere fibrosis panel, n=2, m=1, threshold TPM=0.5, x=200, y=2, and z=2.

### Example 2: Evaluation of Gene Panels

To evaluate the performance of the gene panels, gene set enrichment analysis (GSEA2) as described in Lim et al. (Pacific Symposium on Biocomputing, 2009, 14, 504-515), was modified and calculated using a custom R code as follows. Because each participant was sampled only once in the cross-sectional dataset, a single-sample gene set enrichment analysis was performed with standardized values using z-scores. For each gene in a total universe of size N, where N is the total number of genes whose RNA expression is detected in at least 10% of the samples in the dataset, z-scores were calculated for each sample using “trimmed” means and standard deviations with the largest 1% and smallest 1% of values trimmed from calculation of the mean and standard deviation. Then within each sample, both the z-scores and the additive inverse of the z-scores of the genes were combined into a list of size 2 N, with directional labels for each gene of “up” (for z-scores) or “down” (for the additive inverse of z-scores), and then rank ordered by value. Each gene in the gene panel S with size N_{H} was labeled as trending “up” or “down” with disease stage based on their computed fold changes, resulting in directional gene panel S′. For each directional gene panel S′, where g′ denotes a gene with a directional label,

$${P}_{hit} ( {S}^{′} , i ) = {\sum }_{{g}_{i}^{′} ∈ {S}^{′} , j \leq i} \frac{min ( \vert {z}_{j} \vert , 3 )}{{N}_{R}} , \\ where {N}_{R} = {\sum }_{{g}_{i}^{′} ∈ {S}^{′}} min ( \vert {z}_{j} \vert , 3 ) \\ {P}_{miss} ( {S}^{′} , i ) = {\sum }_{{g}_{i}^{′} ∉ {S}^{′} , j \leq i} \frac{1}{( 2 N - {N}_{H} )}$$

For each directional gene panel S′, an enrichment score ES(S′) was calculated:

$${max}_{1 \leq i \leq 2 N} ( {P}_{hit} ( {S}^{′} , i ) - {P}_{miss} ( {S}^{′} , i ) , 0 )$$

For each S′, a normalized enrichment score

$$NES ( {S}^{′} ) = \frac{ES ( S ′ )}{\frac{1}{k} {\sum }_{1}^{k} ES ( {S}_{r}^{′} )} ,$$

where S′ is one of k random directional gene sets with size N_{H}, was calculated. In this implementation, the mean enrichment score of k=200 random directional gene sets was used as normalization. TS herein refers to the computed NES(S′).

In future clinical trials, each participant may be biopsied before and after drug or surgical treatment in which case genes can be ranked by their fold change instead of z-scores.

### Validation of Gene Panels

The performance of the computed transcriptomic scores was evaluated using either a non-parametric statistical test of Wilcoxon rank-sum test or classification metrics, comparing the transcriptomic scores of samples with histopathology F3+ vs F0/F1 for the fibrosis gene panel and 2+ vs 0 for the inflammation gene panel. Classification metric of precision-recall area-under-the-curve (PRAUC) was computed using all samples with F3+ vs F0/F1 for the fibrosis gene panel and 2+ vs 0 for the inflammation gene panel. 95% confidence intervals for PRAUC were calculated from 2,000 iterations of stratified bootstrapped resampling with replacement, in which the same number of samples as in each category were drawn. Median and 95% confidence interval of the classification metric of receiver operator characteristic area-under-the-curve (ROCAUC) were also calculated from 2,000 iterations of stratified bootstrapped resampling with replacement, in which 100 samples were drawn for each category to obtain a balanced dataset.

To evaluate the performance of smaller subsets of the gene panel, genes in the gene panels were sorted in descending order first by the number of top-200 lists each appeared in and second by median rank across the six lists. The classification performance of the computed transcriptomic score using smaller subsets of the gene panel remained consistent.

Single-cell RNA-seq was used to validate the selection and placement of genes in the panel in either the fibrosis or inflammation panel. Single cells from five healthy and five cirrhotic liver biopsies were clustered into cell types of cholangiocyte cells, mesenchymal cells, endothelial cells, B cells, innate lymphoid cells, mononuclear phagocytes, plasmacytoid dendritic cells, T cells, plasma cells, or hepatocyte cells following the gene signatures in Ramachandran et al. (Nature, 2019, 575, 512-518) using the Python package SCANPY (Wolf at al., Genome Biol, 2018, 19, 15). Single cells labeled as mesenchymal cells were re-clustered into more specific cell subtypes to identify hepatic stellate cells and scar-associated mesenchymal cells. Single cells labeled as mononuclear phagocytes were similarly re-clustered to identify scar-associated macrophages. Genes in the gene panel with detected expression in any immune or fibrotic cell types were identified, and their percent expression across cell types were plotted on a heatmap. Genes were hierarchally clustered and their placement in either fibrosis and/or inflammation panels were labeled, revealing that gene expression in immune and fibrotic cell types from external single-cell RNA-seq largely aligns with gene panels.

### Validation of Gene Panels Methodology

Validation of the gene panel selection methodology was conducted in two manners. First, for the fibrosis gene panel, a gene panel was selected using just a subset of the cohorts and tested on the holdout cohort. Specifically, gene panels were selected using only the GHS and REGN cohorts and tested on the Govaere cohort, selected using only the GHS and Govaere cohorts and tested on the REGN cohort, and selected using only the REGN and Govaere cohorts and tested on the GHS cohort. In each of these cases, genes of interest were limited to those having at least a median TPM value of 0.5 in fibrosis F3 or higher (F3+) or F0 or F1 (F0/F1) in at least one of the two training cohorts. Fold change and PRAUCs were calculated for each of the two training cohorts. The number of times each gene appeared across the four top-200 lists was counted. Genes that appeared in at least 2 of the 4 top 200 gene lists and were statistically significant for fold change at α=0.05 after Benjamini-Hochberg multiple comparison adjustment in both training cohorts were included in the gene panel. Genes in the gene panel were sorted in descending order first by the number of top-200 lists each appeared in and second by median rank across the four lists. The performance of the gene panel was then evaluated using GSEA2 on each of the training and testing cohorts by non-parametric statistical tests of Wilcoxon rank-sum test or classification metrics of PRAUC and ROCAUR. The performances of these gene panels in the testing cohort were comparable to those achieved with the gene panel trained and tested on the entire dataset.

Second, for the fibrosis and inflammation gene panels, performance of a gene panel selected using a training data subset can be tested on a holdout testing data subset. For the fibrosis gene panel, each of the three cohorts were split into a 70% training dataset and a 30% testing dataset within the strata of each fibrosis grade (F3+ vs. F0/F1). Fold change and PRAUCs were calculated for each cohort using only the training split, and genes that appeared in at least 3 of the 6 top 200 gene lists and were statistically significant for fold change at α=0.05 after Benjamini-Hochberg multiple comparison adjustment in at least 2 cohort training splits were included in the panel. For the inflammation gene panel, the GHS and REGN cohorts were split into a 70% training dataset and a 30% testing dataset within the strata of each inflammation grade (2+ vs. 0). Fold change and PRAUCs were calculated for each cohort using only the training split, and genes that appeared in at least 2 of the 4 top 200 gene lists and were statistically significant for fold change at α=0.05 after Benjamini-Hochberg multiple comparison adjustment in both GHS and REGN training splits were included in the panel. For both fibrosis and inflammation, the performance of this gene panel in the holdout testing dataset was comparable to that achieved with the gene panel trained and tested on the entire dataset.

### Flow Diagram of Evaluation of Gene Panels

FIG. 1 (Panel B) shows a flow diagram of the gene panel evaluation. Within each cohort, each subject's transcriptomic score for the enrichment of genes in the gene panel compared to those not in the gene panel is computed. Classification metrics are then computed reflecting the ability of the transcriptomic scores to predict disease class. Subsets of the gene panel are also evaluated to understand the contribution of individual genes in the gene panel towards disease class classification.

Results from Fibrosis Gene Panel

Table 1 shows the fibrosis scoring of liver biopsies from the GHS, REGN, and Govaere cohorts.

TABLE 1

| Fibrosis | GHS | REGN | Govaere |
|---|---|---|---|
| F0 | 725 | 1079 | 46 |
| F1 | 175 | 347 | 48 |
| F2 | 38 | 51 | 54 |
| F3 | 33 | 19 | 54 |
| F34, F4 | 16 | 23 | 14 |
| Unknown | 6 | 16 | 0 |

Most patients in the GHS and REGN cohorts had lower fibrosis scores whereas the distribution of scores for the patients in the Govaere cohort was more evenly distributed.

Table 2 shows the fibrosis scoring of liver biopsies from the GHS, REGN, and Govaere cohorts with paired histopathology showing steatosis and for the GHS and REGN cohorts with fibrosis and lobular inflammation histopathology values and for the Govaere cohort with fibrosis histopathology values.

TABLE 2

| Fibrosis | GHS | REGN | Govaere |
|---|---|---|---|
| F0 | 472 | 790 | 38 |
| F1 | 169 | 334 | 47 |
| F2 | 38 | 49 | 53 |
| F3 | 32 | 19 | 54 |
| F34, F4 | 15 | 22 | 14 |

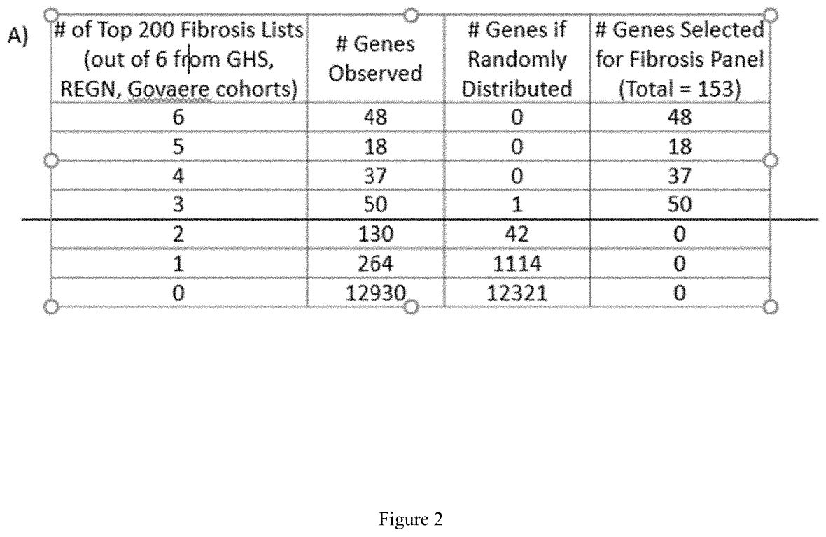

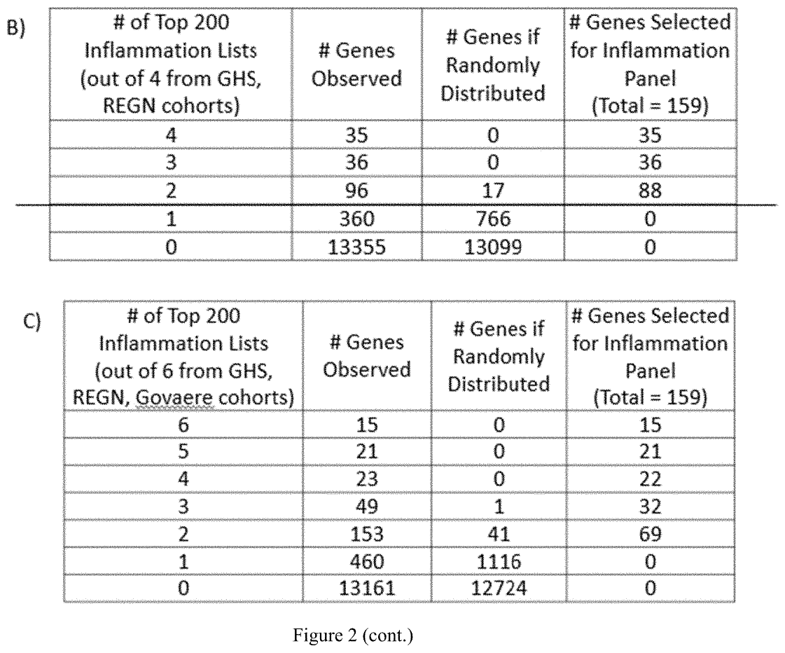

FIG. 2 (Panel A) shows distribution of the number of times a given gene appeared across the six top-200 gene fibrosis ranked lists, the expected distribution of the number of times a gene appears across six randomly selected lists of 200 genes, and the distribution of the number of times each gene in the selected fibrosis gene panel appeared across the six top-200 gene fibrosis ranked lists. The six fibrosis ranked lists are one list of fold change and one of precision-recall area under the curve, comparing fibrosis stage F3 and higher versus fibrosis stage F0 and F1, from each of the three GHS, REGN, and Govaere cohorts. Genes that appeared in at least three out of the six top-200 gene fibrosis ranked lists, as indicated by the horizontal black threshold line, and were statistically significant for fold change in at least two of the cohorts were selected to be in the fibrosis gene panel. The 153 genes appearing in at least three lists do so at much greater numbers than would have been expected if genes were randomly distributed across the lists. The following genes were found in the top 200 genes of three or more of the fibrosis lists and were statistically significant for fold change in at least 2 cohorts, in ranked order: STMN2, FAP, ITGBL1, MOXD1, COL10A1, NALCN, SCTR, EFEMPI, CLIC6, MMP7, THY1, LOXL1, MDFI, LTBP2, VTCN1, LUM, CLDN11, CFAP221, CFTR, DCDC2, EPCAM, ADRA2A, LAMC3, AEBP1, PAPLN, RASL11B, CDH6, PTGDS, LOXL4, BHLHE22, CPZ, CD24, FBLN5, DPT, BICC1, WNT4, LRRC1, LAMA2, PODN, RAB25, SPINTi, TMPRSS3, DKK3, SOX9, EPHA3, MFAP4, GPC4, SNAP25, GJA5, UBD, DTNA, LEF1, THBS2, PLCXD3, CTHRC1, SUSD2, SMOC2, SOD3, SPON1, PDZK1IP1, F3, MMP2, MFAP2, C7, CKMT2, CLDN10, CXCL6, AKR1B10, PHLDA3, COMP, CD40LG, PTK7, CCDC80, SEZ6L2, COL16A1, AQP1, GSN, GEM, NELL2, PAQR5, TYMS, LXN, TRAC, PLPP4, AP1M2, GPRC5B, VEPH1, DPPA4, RGS4, SPP1, EGFLAM, OLR1, MGP, SLC7A6, BEX2, SIT1, ANO9, DPYSL3, CD1E, ANTXR1, BOC, LAYN, PDGFD, CCL21, HKDC1, CD5, CCL19, GPC3, OMG, FBLN2, TRAT1, SEMA3G, KRT7, ANKRD29, COL5A1, IGLC3, UBASH3A, PDGFA, NFASC, CCR6, LGR6, WFDC2, NPNT, VWF, MUC6, COL1A2, SLAMF1, IGHG1, CXCR3, F13A1, TACSTD2, SIRPG, STMN3, IGFBP7, CXCL1, CCR2, CCDC146, CTSK, COL1A1, HSPB2, RRAD, COL3A1, BACE2, ZMAT3, PCYOX1L, EEF1A2, CHI3L1, FXYD2, CCL20, CH25H, MAT1A, SEPTIN8, and CXCL8. All of these genes were upregulated with fibrosis histopathology except for DPPA4, EGFLAM, and MAT1A that were downregulated.

A heatmap of single cell RNA-seq expression was generated for genes in the fibrosis gene panel across cell types (data not shown). The following genes in the fibrosis gene panel were expressed in more than 50% of cholangiocyte cells from cirrhotic livers: AQP1, BICC1, CD24, CFTR, CLDN10, CXCL6, DCDC2, EPCAM, FXYD2, GSN, KRT7, MMP7, PDZK1IP1, SOD3, SOX9, SPINTi, SPP1, TACSTD2, and WFDC2. The following genes in the fibrosis gene panel were expressed in more than 50% of mesenchymal cells from cirrhotic livers: AEBP1, CCDC80, COL1A1, COL1A2, COL3A1, GSN, IGFBP7, MFAP4, MGP, and SOD3. The following genes in the fibrosis gene panel were expressed in more than 50% of endothelial cells from cirrhotic livers: AQP1, GSN, IGFBP7, MGP, and VWF. The following genes in the fibrosis gene panel were expressed in more than 50% of hepatic stellate cells from cirrhotic livers: COL1A2, GEM, GSN, IGFBP7, MGP, and SOD3. The following genes in the fibrosis gene panel were expressed in more than 50% of scar-associated mesenchymal cells from cirrhotic livers: AEBP1, C7, CCDC80, COL1A1, COL1A2, COL3A1, COL5A1, DPT, EFEMPI, FBLN5, GSN, IGFBP7, IGLC3, LUM, LXN, MFAP4, MGP, MMP2, PTGDS, SOD3, and THY1.

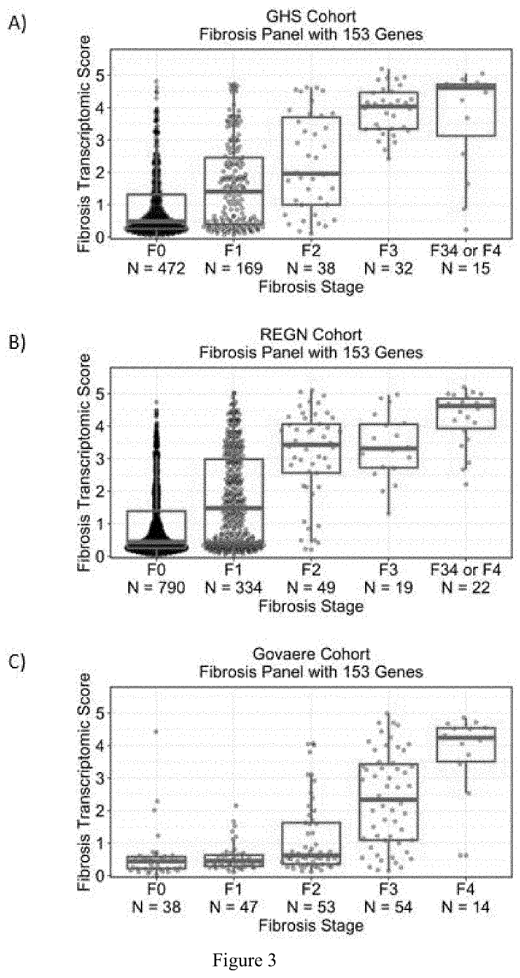

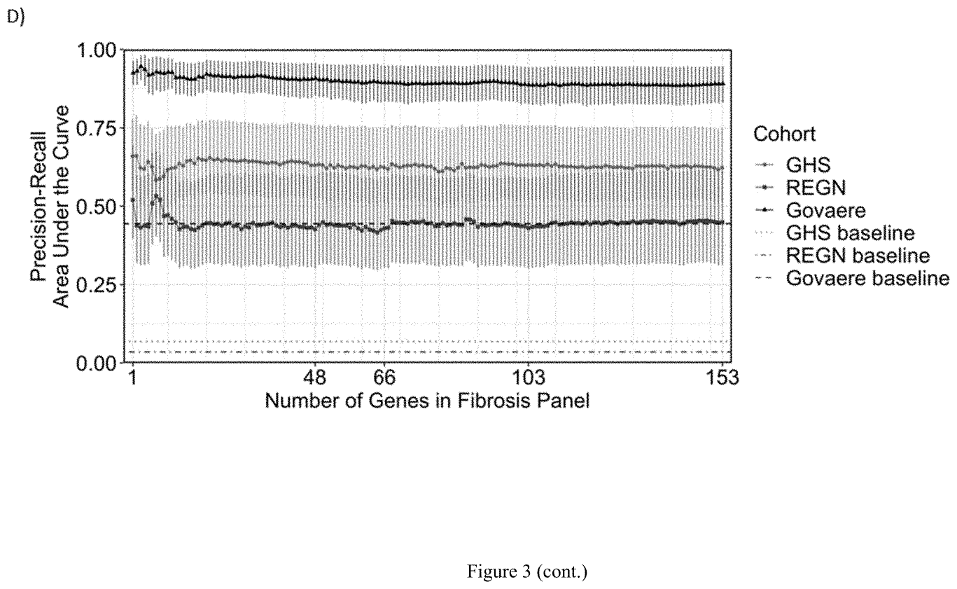

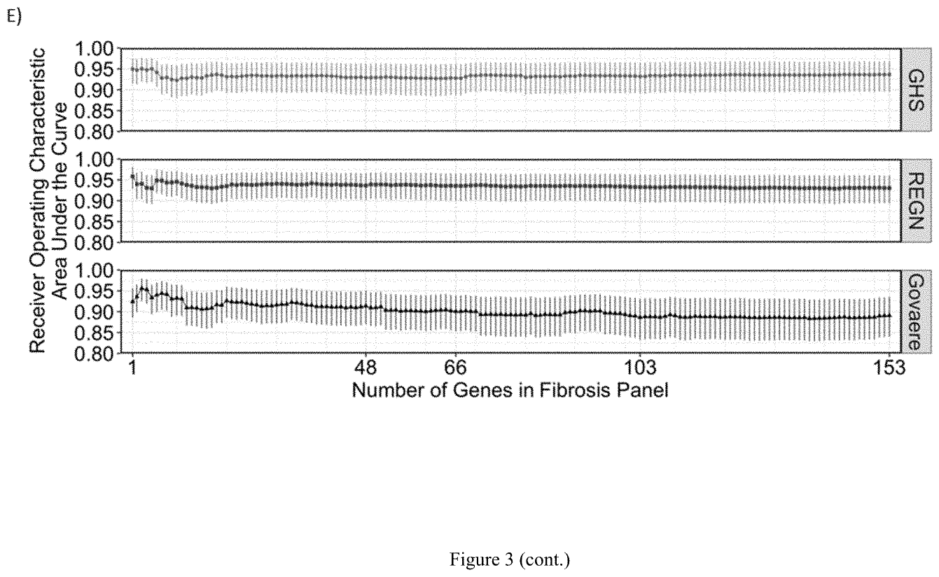

FIG. 3 (Panels A, B, and C) shows variation of fibrosis transcriptome score with fibrosis stage across the GHS, REGN, and Govaere cohorts, respectively. For each cohort, the fibrosis transcriptome score trended with the fibrosis score determined by histopathology. Table 3 and Table 4 show the significance of comparisons of fibrosis transcriptomic scores between different fibrosis disease stages by Wilcoxon rank sum tests. FIG. 3 (Panels D and E) shows variation of PRAUC and ROCAUC values, respectively, with the number of genes studied, where the gene panel is incrementally increased by one gene next in rank order of the gene panel from a single gene to the full panel size of 153 genes, and demonstrates that if subsets were used to generate the fibrosis transcriptome score, additional genes from the full panel did not substantially change the PRAUC or ROCAUC values. Error bars represent the 95% confidence interval of AUC calculated from stratified bootstrapping resampling with replacement. In Panel D, horizontal dotted lines represent the baseline PRAUC of the null random model for each cohort, which is the fraction of “positive” subjects out of total subjects. In Panel E, the baseline ROCAUC of the null random model is 0.5. For all three cohorts for each gene panel size, fibrosis transcriptomic scores classify fibrosis stage significantly better than the null random model (p<0.001) in both PRAUC and ROCAUC.

TABLE 3

| | | | p-value | | | |
|---|---|---|---|---|---|---|
| | | | F0F1 vs | p-value | p-value | p-value |
| Cohort | Gene Panel | Split | F3+ | F0 vs F1 | F1 vs F2 | F2 vs F3 |
| GHS | Fibrosis All | All | 2.1E−23 | 4.4E−10 | 9.5E−03 | 1.3E−06 |
| GHS | Fibrosis GHS | All | 3.7E−23 | 2.2E−10 | 5.5E−03 | 2.2E−06 |
| | REGN | | | | | |
| GHS | Fibrosis GHS | All | 9.6E−23 | 2.4E−10 | 1.7E−02 | 5.4E−07 |
| | Govaere | | | | | |
| GHS | Fibrosis REGN | All | 4.4E−23 | 2.9E−10 | 1.3E−02 | 1.7E−06 |
| | Govaere | | | | | |
| GHS | Fibrosis 70% | Train | 1.8E−17 | 3.5E−07 | 4.6E−02 | 6.5E−06 |
| | Train Split | | | | | |
| GHS | Fibrosis 70% | Test | 4.2E−07 | 6.8E−04 | 1.1E−01 | 1.1E−01 |
| | Train Split | | | | | |
| REGN | Fibrosis All | All | 8.0E−21 | 1.1E−20 | 1.1E−08 | 1.2E−02 |
| REGN | Fibrosis GHS | All | 2.2E−21 | 6.8E−22 | 8.8E−09 | 4.0E−03 |
| | REGN | | | | | |
| REGN | Fibrosis GHS | All | 5.6E−20 | 1.7E−18 | 3.7E−08 | 1.6E−02 |
| | Govaere | | | | | |
| REGN | Fibrosis REGN | All | 8.7E−22 | 9.4E−20 | 1.4E−08 | 2.8E−03 |
| | Govaere | | | | | |
| REGN | Fibrosis 70% | Train | 2.5E−15 | 2.8E−10 | 8.4E−08 | 8.8E−02 |
| | Train Split | | | | | |
| REGN | Fibrosis 70% | Test | 4.1E−07 | 4.6E−09 | 4.7E−02 | 1.9E−02 |
| | Train Split | | | | | |

TABLE 4

| | | | p-value | | | | |
|---|---|---|---|---|---|---|---|
| | Gene | | F0F1 vs | p-value | p-value | p-value | p-value |
| Cohort | Panel | Split | F3+ | F0 vs F1 | F1 vs F2 | F2 vs F3 | F3 vs F4 |
| Govaere | Fibrosis | All | 1.4E−16 | 1.8E−01 | 7.1E−03 | 1.7E−05 | 2.4E−03 |
| | All | | | | | | |
| Govaere | Fibrosis | All | 1.4E−15 | 4.8E−02 | 2.5E−03 | 1.9E−04 | 5.6E−03 |
| | GHS | | | | | | |
| | REGN | | | | | | |
| Govaere | Fibrosis | All | 1.4E−17 | 1.1E−01 | 1.3E−03 | 2.6E−05 | 1.9E−03 |
| | GHS | | | | | | |
| | Govaere | | | | | | |
| Govaere | Fibrosis | All | 2.8E−17 | 7.2E−02 | 2.8E−03 | 1.6E−05 | 2.7E−03 |
| | REGN | | | | | | |
| | Govaere | | | | | | |
| Govaere | Fibrosis | Train | 3.6E−12 | 2.5E−01 | 3.5E−04 | 2.6E−03 | 5.2E−03 |
| | 70% | | | | | | |
| | Train | | | | | | |
| | Split | | | | | | |
| Govaere | Fibrosis | Test | 3.8E−05 | 5.2E−01 | 5.7E−01 | 2.5E−03 | 2.5E−01 |
| | 70% | | | | | | |
| | Train | | | | | | |
| | Split | | | | | | |

Results from Inflammation Gene Panel

Table 5 shows the inflammation scoring of liver biopsies from the GHS and REGN cohorts.

TABLE 5

| Inflammation | GHS | REGN |
|---|---|---|
| 0 | 676 | 998 |
| 1 | 250 | 484 |
| 2 | 60 | 43 |
| 3 | 2 | 4 |
| Unknown | 5 | 16 |

Table 6 shows the inflammation scoring of liver biopsies from the GHS and REGN cohorts, with paired histopathology showing steatosis and with fibrosis and lobular inflammation histopathology values.

TABLE 6

| Inflammation | GHS | REGN |
|---|---|---|
| 0 | 419 | 689 |
| 1 | 245 | 479 |
| 2 | 60 | 42 |
| 3 | 2 | 4 |

FIG. 2 (Panel B) shows distribution of the number of times a given gene appeared across the four top-200 gene inflammation ranked lists, the expected distribution of the number of times a gene appears across four randomly selected lists of 200 genes, and the distribution of the number of times each gene in the selected inflammation gene panel appeared across the four top-200 gene inflammation ranked lists. The four inflammation ranked lists are one list of fold change and one of precision-recall area under the curve, comparing inflammation stage 2 and higher versus inflammation stage 0, from each of the two GHS and REGN cohorts. Genes that appeared in at least two out of the four top-200 gene inflammation ranked lists, as indicated by the horizontal black threshold line, and were statistically significant for fold change in both GHS and REGN cohorts were selected to be in the inflammation gene panel. The 159 genes appearing in at least two lists do so at much greater numbers than would have been expected at random. FIG. 2 (Panel C) shows distribution of the number of times a given gene appeared across the six top-200 gene inflammation ranked lists, the expected distribution of the number of times a gene appears across six randomly selected lists of 200 genes, and the distribution of the number of times each gene in the selected inflammation gene panel appeared across the six top-200 gene inflammation ranked lists. The six inflammation ranked lists are one list of fold change and one of precision-recall area under the curve, comparing inflammation stage 2 and higher versus inflammation stage 0, from each of the three GHS, REGN, and Govaere cohorts. The following genes were found in the top 200 genes of two or more of the four inflammation lists from the GHS and REGN cohorts and were statistically significant for fold change in both GHS and REGN cohorts, in ranked order: LPL, STMN2, TREM2, FABP4, COMP, CAPG, SPP1, LOXL4, FABP5, THY1, EMILIN2, SLAMF8, BCAT1, CD300LB, CLDN11, DTNA, OLR1, MMP9, SPATA21, UBD, ITGBL1, CCL22, C15orf48, LGALS3, CXCL10, LTBP2, CPZ, KCNN4, COL1A1, DHRS9, LYZ, EFEMPI, THBS2, RTN1, CD24, IL32, HS3ST2, MOXD1, GPNMB, COL3A1, TTC9, CENPV, LOXL1, PDGFA, SCTR, COL1A2, CCL20, LAMC3, PAPLN, RAB7B, AEBP1, TP5313, MDFI, LUM, RGS10, CLIC6, RASL10B, LAIR1, PLXNC1, ALOX5AP, PODN, LSP1, CD52, JAK3, VCAN, TNFRSF4, WNT4, KRT23, LRRC1, LAMA2, SNAP25, CD37, ITGAX, MTHFD2, CMYA5, DNAJC5B, PTPN7, DUSP8, PTGDS, MICAL1, MRAS, PRAMEF10, CCL17, DGKA, GEM, CD1E, CDH6, RRAD, MMP7, HAPLN3, GAPT, B3GNT5, CD5, NELL2, PBX4, SLC7A6, BHLHE22, FSTL3, CD48, CCR7, IL411, SLC1A7, SLC2A14, ADAM28, KLHL29, LAMA3, ACADSB, CCR5, GJA5, CD40LG, KRTCAP3, FCAMR, DNAJC12, SIT1, CYP2C19, EPHA3, RPS6KA1, FBLN2, WFDC2, CD6, CXCR3, VIL1, CD28, CHIT1, TRAF1, CD2, ANO9, SLC16A3, ABR, PDCD1, CD96, TRIM31, SUSD2, SAMD11, PLD4, CCR2, LRFN4, FGR, CACNB1, TMEM164, GRAP2, GABRE, MMP14, SIRPG, LGALS2, NCF2, CARMIL2, MACO1, SLCO1A2, MS4A14, PRAMEF33, TRPM2, LCK, EGFLAM, LILRB3, AQP8, GPR174, BATF, and MT1B. All of these genes were upregulated with inflammation histopathology except for CENPV, RASL10B, CMYA5, ACADSB, KRTCAP3, DNAJC12, CYP2C19, VIL1, MACO1, SLCO1A2, EGFLAM, and MT1B that were downregulated.

A heatmap of single cell RNA-seq expression was generated for genes in the inflammation gene panel across cell types (data not shown). The following genes in the inflammation gene panel were expressed in more than 25% of B cells from cirrhotic livers: ADAM28, ALOX5AP, CAPG, CCR7, CD24, CD37, CD48, CD52, GAPT, IL32, LSP1, and SIT1. The following genes in the inflammation gene panel were expressed in more than 25% of innate lymphoid cells from cirrhotic livers: ALOX5AP, CD2, CD37, CD48, CD52, CD96, FGR, IL32, LCK, LSP1, and PTPN7. The following genes in the inflammation gene panel were expressed in more than 25% of mononuclear phagocytes from cirrhotic livers: ALOX5AP, CAPG, CD37, CD48, CD52, FABP5, FGR, GAPT, IL32, ITGAX, LAIR1, LGALS2, LGALS3, LILRB3, LSP1, LYZ, MTHFD2, NCF2, PLD4, RGS10, SLC16A3, and VCAN. The following genes in the inflammation gene panel were expressed in more than 25% of plasmacytoid dendritic cells from cirrhotic livers: ALOX5AP, CAPG, CCR2, CD37, CD48, CD52, CXCR3, FABP5, GAPT, IL32, LAIR1, LSP1, LYZ, PLD4, PTGDS, RGS10, and SIT1. The following genes in the inflammation gene panel were expressed in more than 25% of T cells from cirrhotic livers: ALOX5AP, CD2, CD37, CD40LG, CD48, CD52, CD6, CD96, CXCR3, IL32, LCK, LSP1, PTPN7, RGS10, and SIT1. The following genes in the inflammation gene panel were expressed in more than 25% of scar-associated macrophages from cirrhotic livers: ADAM28, ALOX5AP, BCAT1, CAPG, CD37, CD48, CD52, DHRS9, FABP5, GPNMB, IL32, ITGAX, LAIR1, LGALS2, LGALS3, LILRB3, LSP1, LYZ, MTHFD2, NCF2, PLD4, RGS10, SLAMF8, SLC16A3, and TREM2.

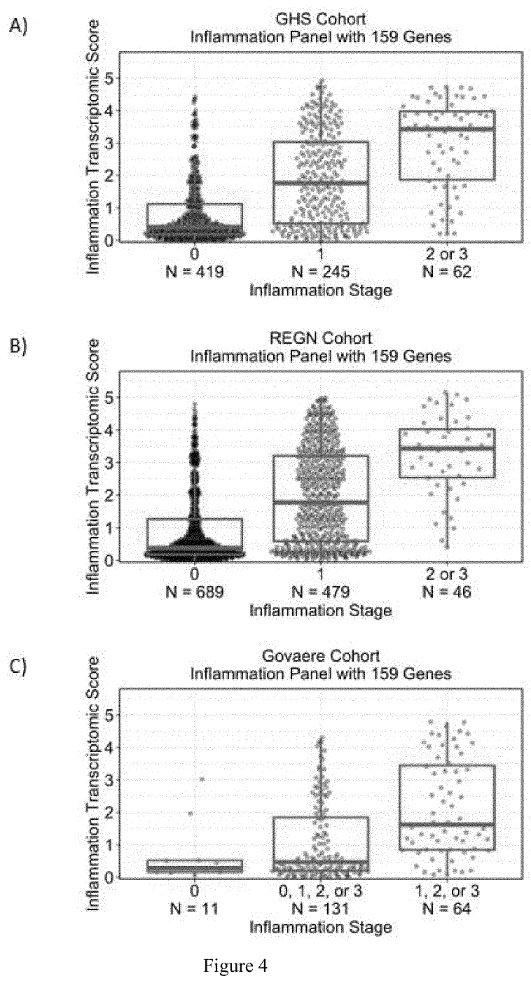

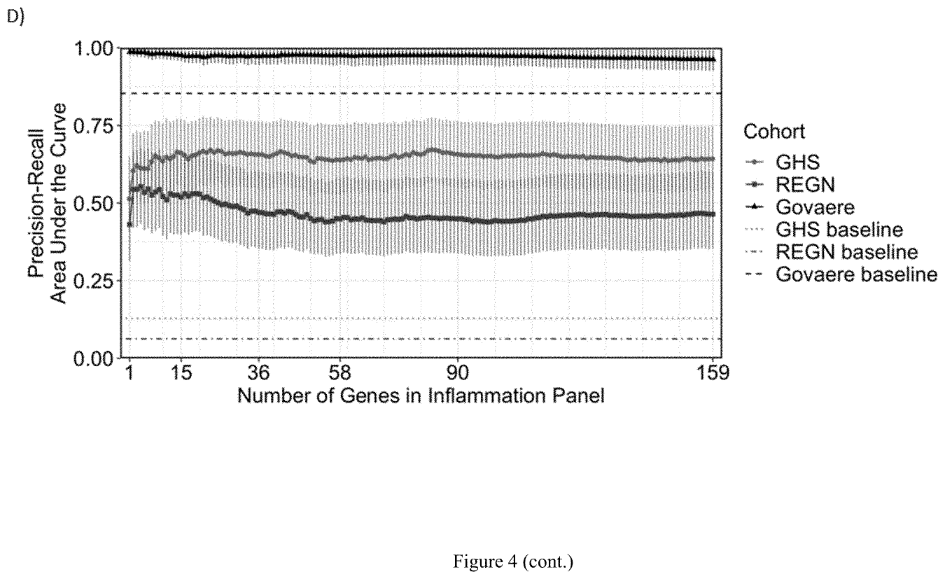

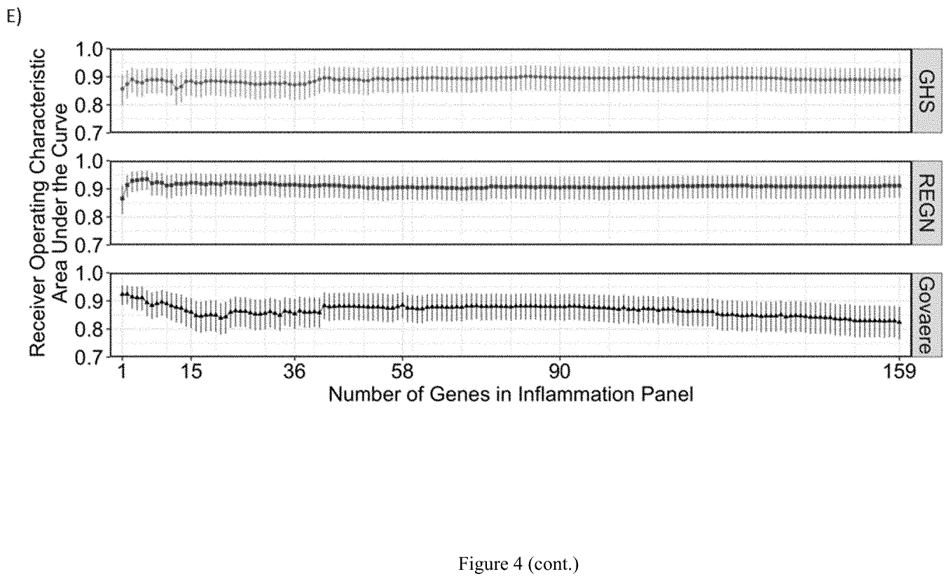

FIG. 4 (Panels A, B, and C) shows variation of inflammation transcriptome score with inflammation stage across the GHS, REGN, and Govaere cohorts, respectively. For each cohort, the inflammation transcriptome score trended with the inflammation score determined by histopathology. Table 7 and Table 8 show the significance of comparisons of inflammation transcriptomic scores between different inflammation disease stages by Wilcoxon rank sum tests. For the Govaere cohort, the “negative” cases are the 11 samples identified to have an inflammation value of 0, and the “positive” cases are the 64 samples identified to likely have an inflammation value of 2+(of which 6 samples had an inflammation value of 1, and 58 samples had an inflammation value of 2+). The other 131 non-control samples in the Govaere cohort are shown in the middle boxplot of FIG. 4 (Panel C). FIG. 4 (Panels D and E) shows variation of PRAUC and ROCAUC values, respectively, with the number of genes studied, where the gene panel is incrementally increased by one gene next in rank order of the gene panel from a single gene to the full panel size of 159 genes, and demonstrates that if subsets were used to generate the inflammation transcriptome score, additional genes from the full panel did not substantially change the PRAUC or ROCAUC values. Error bars represent the 95% confidence interval of AUC calculated from stratified bootstrapping resampling with replacement. In Panel D, horizontal dotted lines represent the baseline PRAUC of the null random model for each cohort, which is the fraction of “positive” subjects out of total subjects. In Panel E, the baseline ROCAUC of the null random model is 0.5. For all three cohorts for each gene panel size, inflammation transcriptomic scores classify inflammation stage significantly better than the null random model (p<0.001) in both PRAUC and ROCAUC.

TABLE 7

| | | | p-value | p-value | p-value |
|---|---|---|---|---|---|
| Cohort | Gene Panel | Split | 0 vs 2+ | 0 vs 1 | 1 vs 2+ |
| GHS | Inflammation All | All | 5.5E−23 | 1.1E−25 | 2.8E−07 |
| GHS | Inflammation 70% Train Split | Train | 1.1E−15 | 2.7E−17 | 8.2E−06 |
| GHS | Inflammation 70% Train Split | Test | 7.0E−10 | 4.8E−10 | 2.5E−03 |
| REGN | Inflammation All | All | 1.2E−20 | 1.0E−42 | 8.0E−08 |
| REGN | Inflammation 70% Train Split | Train | 5.3E−15 | 2.5E−31 | 2.3E−05 |
| REGN | Inflammation 70% Train Split | Test | 6.1E−07 | 3.2E−13 | 3.3E−03 |

TABLE 8

| | | | p-value 0 vs |
|---|---|---|---|
| Cohort | Gene Panel | Split | Likely 2+ |
| Govaere | Inflammation All | All | 6.9E−04 |
| Govaere | Inflammation 70% Train Split | All | 1.4E−04 |

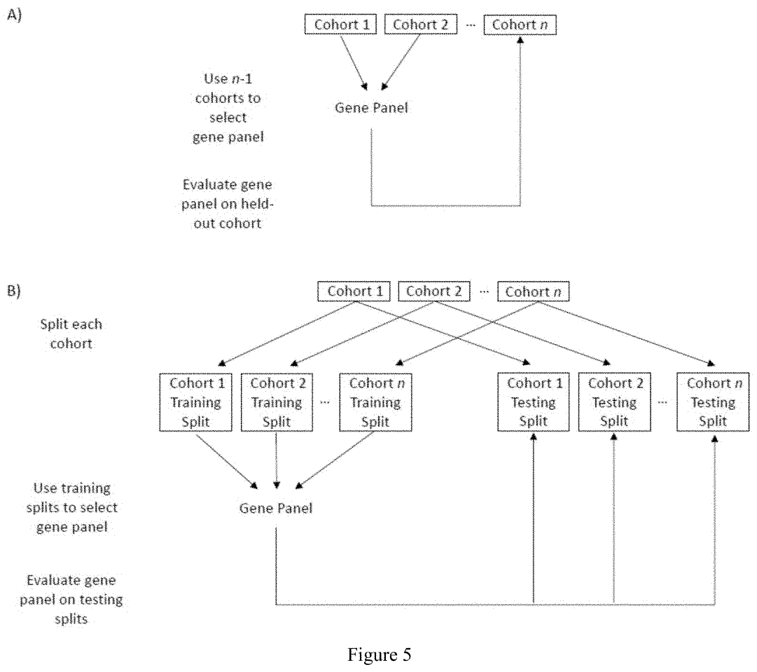

FIG. 5 (Panels A and B) shows two methods for testing the robustness of the gene panel selection methodology by training the gene panel from a subset of the available subjects and testing the gene panel on the other subjects. FIG. 5 (Panel A) shows a methodology of using n−1 of the available n cohorts to select the gene panel and evaluating the performance of the gene panel on the held-out cohort. FIG. 5 (Panel B) shows a methodology of splitting each cohort into a training split and a testing split, using the training splits from each cohort to select the gene panel, and evaluating performance of the gene panel on the testing split from each cohort.

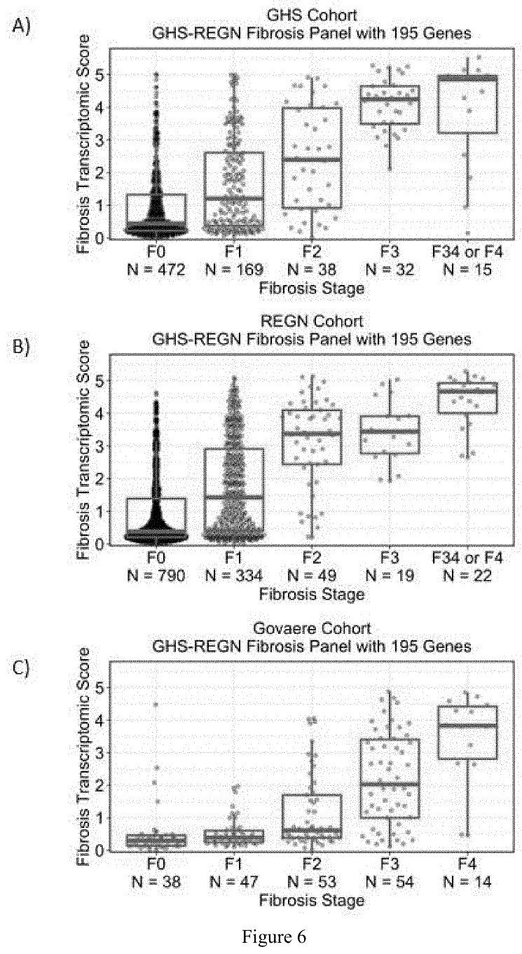

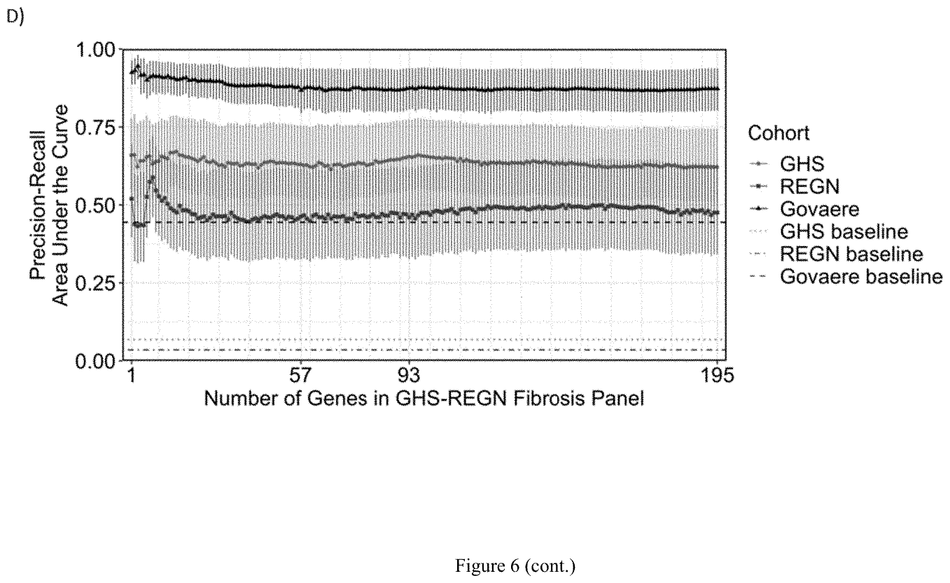

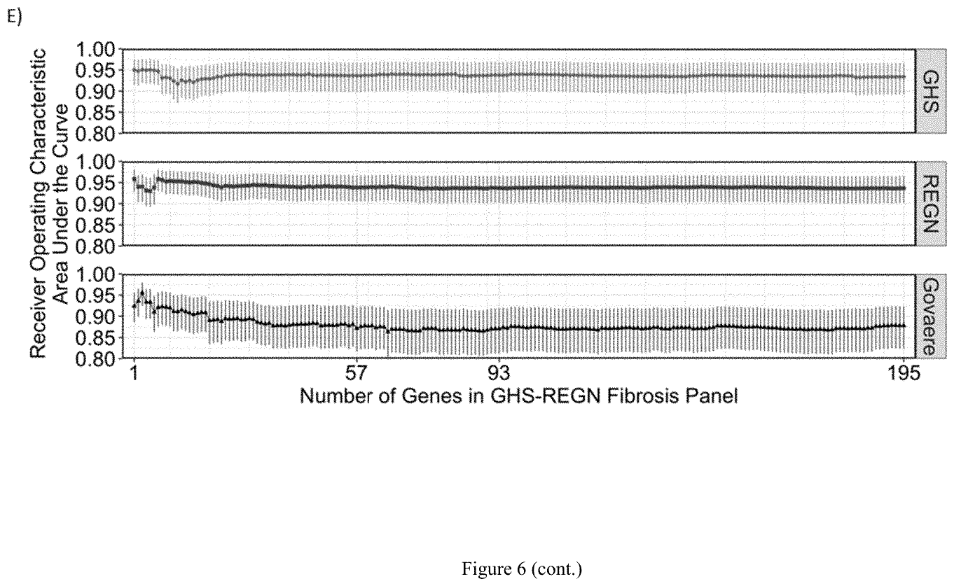

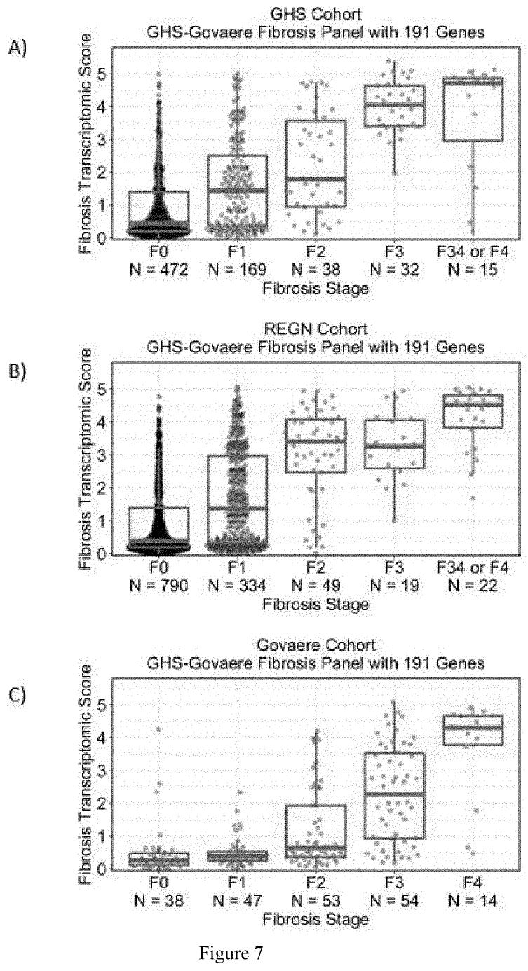

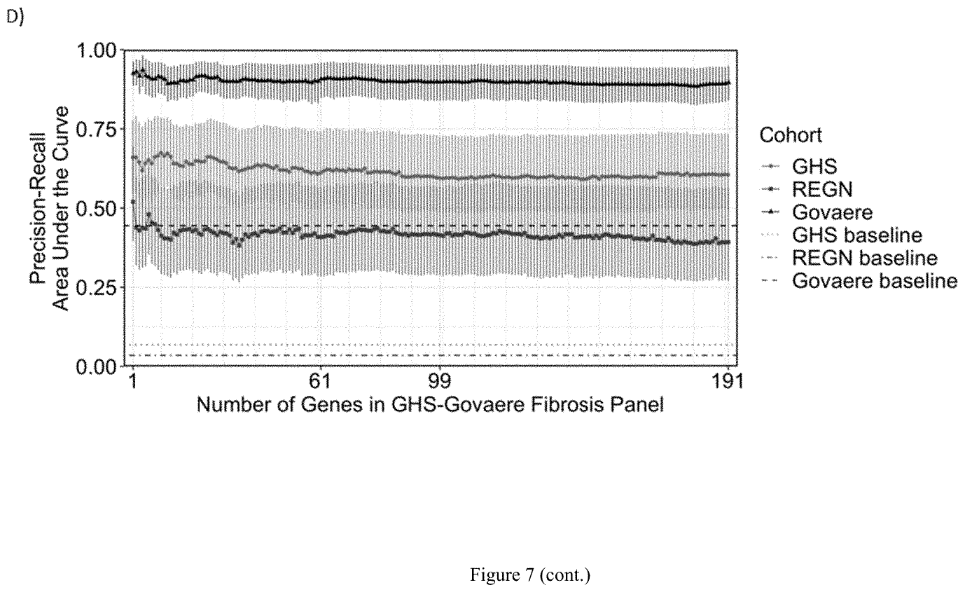

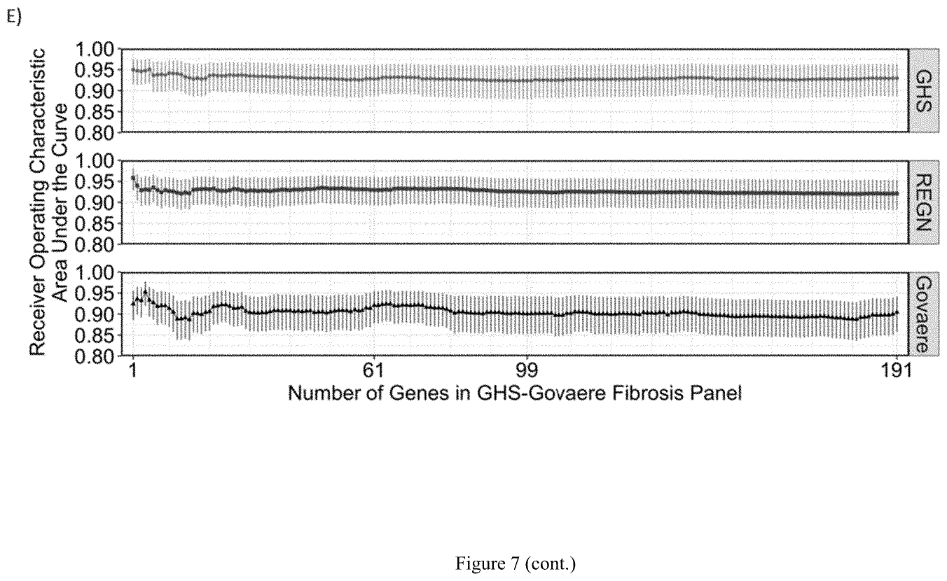

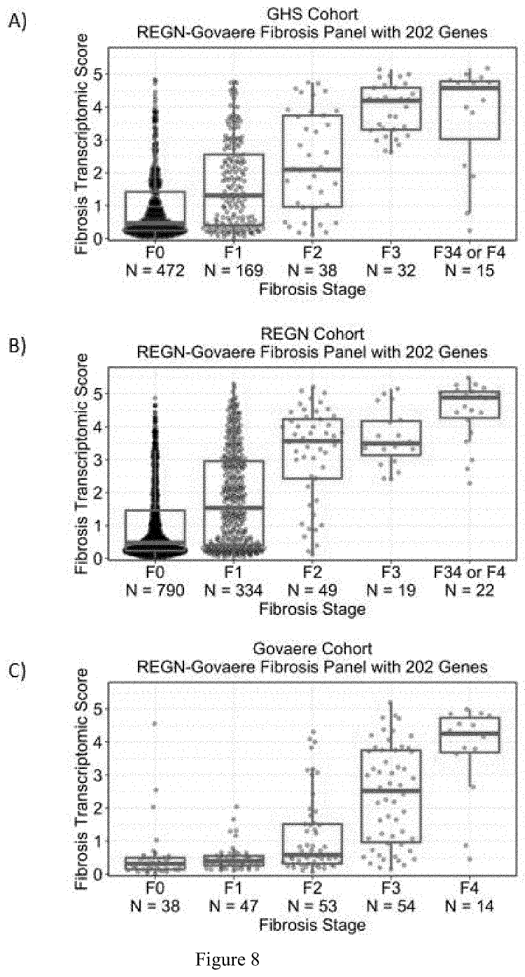

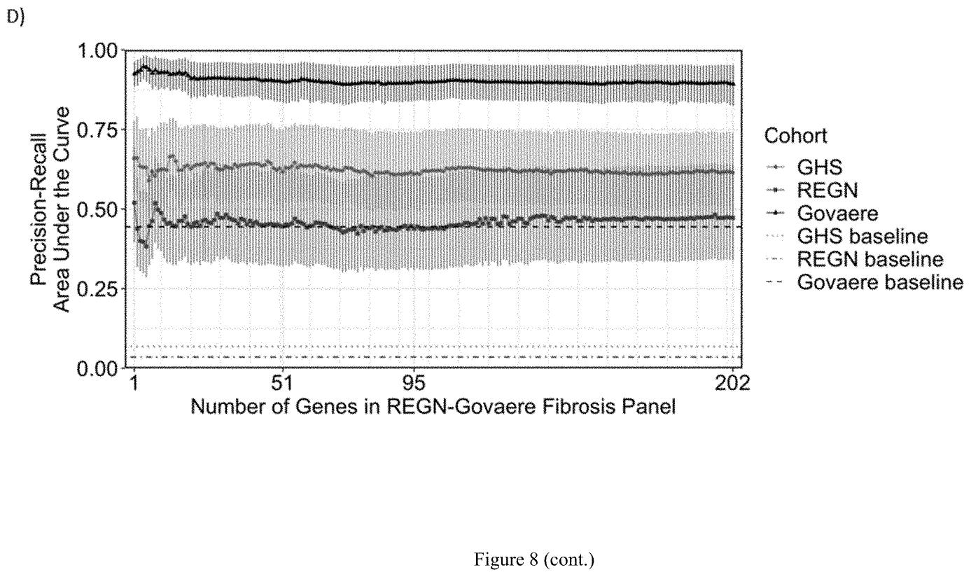

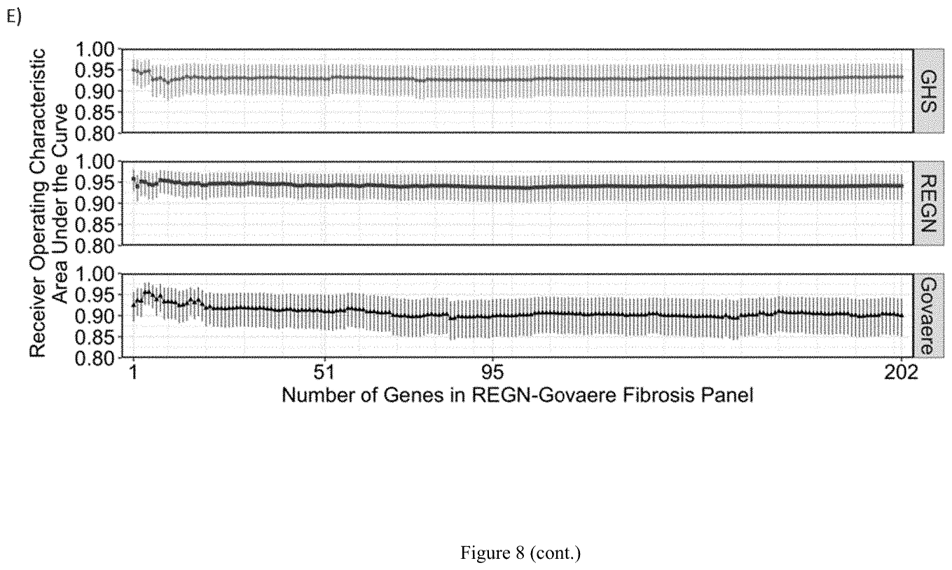

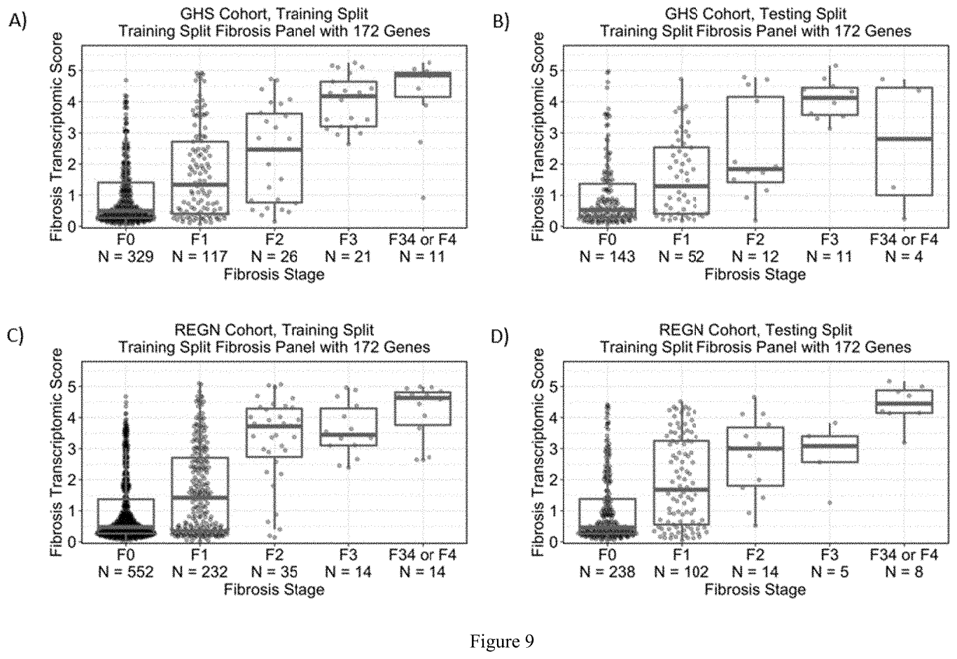

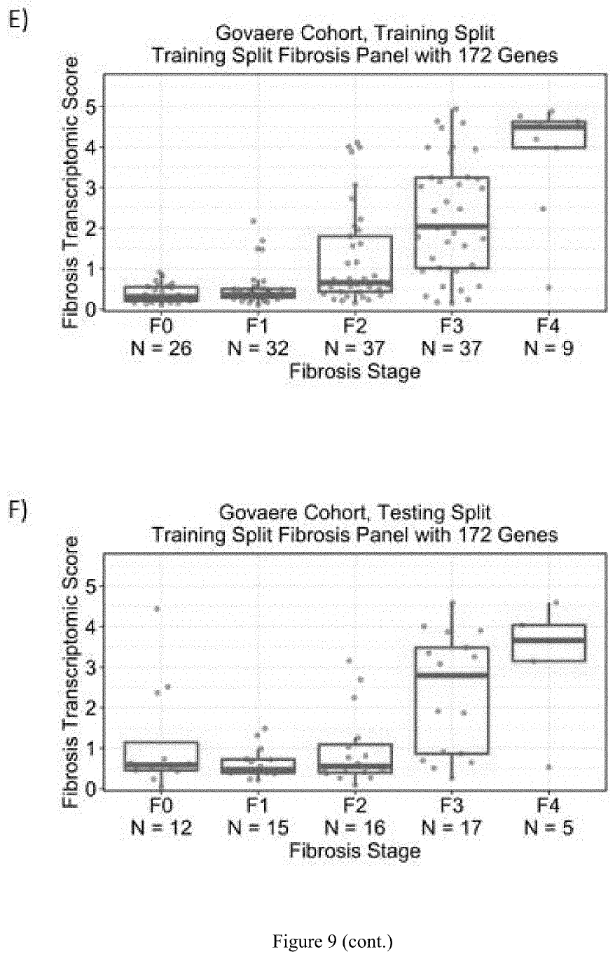

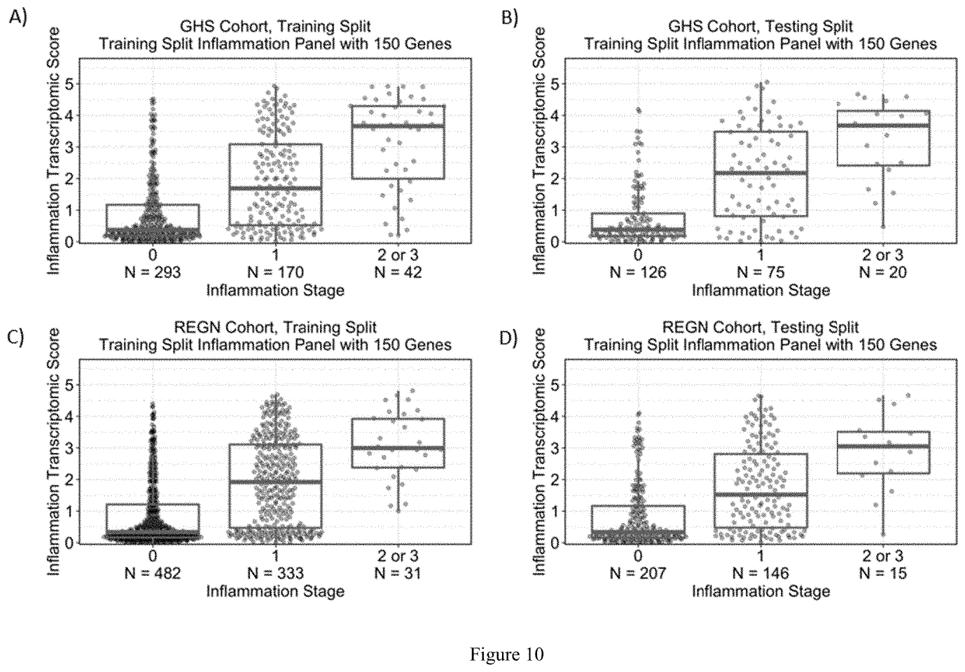

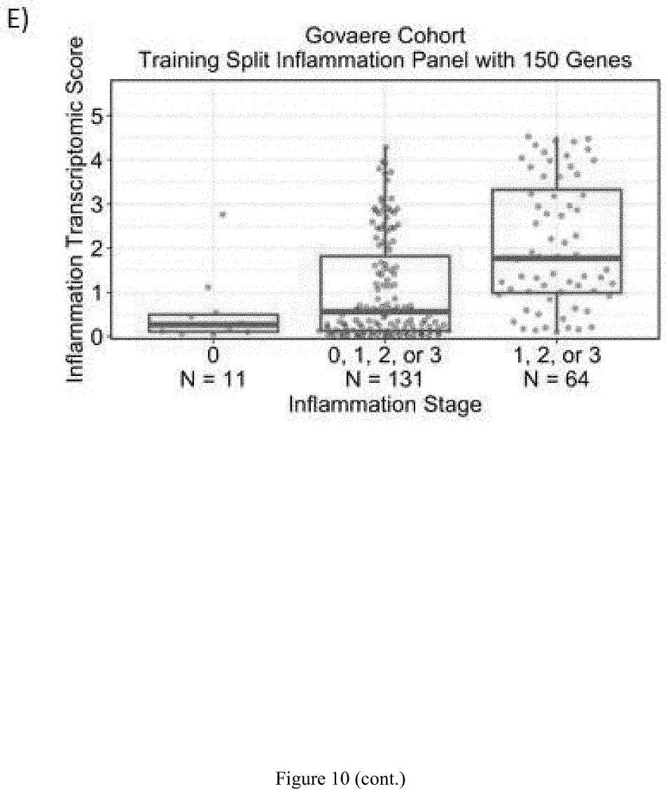

FIG. 6 (Panels A, B, C, D, and E), FIG. 7 (Panels A, B, C, D, and E), and FIG. 8 (Panels A, B, C, D, and E), FIG. 9 (Panels A, B, C, D, E, and F), and FIG. 10 (Panels A, B, C, D, and E) demonstrate the robustness of the gene panel selection methodology. For the fibrosis gene panel, a gene panel was selected using just a subset of the cohorts and tested on the holdout cohort. Specifically, a gene panel was selected using only the GHS and REGN cohorts and tested on the Govaere cohort (FIG. 6), selected using only the GHS and Govaere cohorts and tested on the REGN cohort (FIG. 7), and selected using only the REGN and Govaere cohorts and tested on the GHS cohort (FIG. 8). For the fibrosis gene panel, a gene panel derived from only the 70% training splits from the GHS, REGN, and Govaere cohorts was tested on the training and testing split from the GHS, REGN, and Govaere cohorts (FIG. 9). For the inflammation gene panel, a gene panel derived from only the 70% training splits from the GHS and REGN cohorts was tested on the training and testing split from the GHS and REGN cohorts and on the entire Govaere cohorts (FIG. 10). The inflammation categories of the Govaere cohort in FIG. 10 (Panel E) are the same as in FIG. 4 (Panel C). For the fibrosis gene panels selected using only the GHS and REGN cohorts, the GHS and Govaere cohorts, the REGN and Govaere cohorts, and the 70% training splits from all three cohorts, the fibrosis transcriptome score trended with the fibrosis score determined by histopathology and fibrosis transcriptomic scores classify fibrosis stage significantly better than the null random model (p<0.001) in both PRAUC and ROCAUC. For the inflammation gene panels selected using only the 70% training splits from the GHS and REGN cohorts, the inflammation transcriptome score trended with the inflammation score determined by histopathology and inflammation transcriptomic scores classify inflammation stage significantly better than the null random model (p<0.001) in both PRAUC and ROCAUC.

The following fibrosis gene panel was selected using only the GHS and REGN data, in ranked order: STMN2, FAP, ITGBL1, COL10A1, MOXD1, GJA5, NALCN, SCTR, EFEMPI, MMP7, CLIC6, DCDC2, CLDN11, VTCN1, AEBP1, CFTR, LUM, LTBP2, PAPLN, LEF1, THY1, MDFI, LOXL1, PHLDA3, ADRA2A, CPZ, LAMC3, RASL11B, PTGDS, BHLHE22, EPCAM, SUSD2, CDH6, BICC1, PODN, CD40LG, FBLN5, SOD3, WNT4, LAMA2, EPHA3, CD24, NELL2, RAB25, TYMS, DKK3, TMPRSS3, TRAC, LRRC1, SPINTi, DPT, SOX9, CKMT2, MFAP4, ANTXR1, GPC4, SIT1, CTHRC1, DTNA, COMP, IGLC3, FBLN2, CFAP221, SMOC2, TRAT1, OMG, THBS2, CCDC80, UBD, CD5, F13A1, SPON1, MFAP2, LOXL4, PLCXD3, SEZ6L2, IGHG1, LXN, MMP2, SIRPG, PDZK1IP1, CXCR3, CCR6, VEPH1, SLAMF1, UBASH3A, AP1M2, SPP1, STMN3, CCR2, ZMAT3, ANO9, SNAP25, CCL21, LBH, EGFLAM, TNFRSF17, F3, LAYN, IL7R, NTS, TTC9, CXCL6, CD27, CLDN10, IGLL5, TESPA1, DPYSL3, IGLC1, C7, PCNX2, PTK7, IGLC2, AQP1, IGKC, TRBC2, ADAM28, PDGFD, NPNT, SAMD11, SLC1A7, PLPP4, MZB1, GSN, CD3D, PAQR5, CCL19, TRBC1, CD96, VWF, KCNN4, CLEC4M, MUC6, CD28, BHLHE41, GPRC5B, TMEM159, SLC7A6, CD2, IGHA1, MS4A1, RNASE1, CCDCl46, EDN2, COLA1, AKR1B10, CTSK, TNFRSF13B, DCN, MGP, IGHG3, SCRN1, TC2N, HKDC1, MAP9, EEF1A2, POU2AF1, CD1E, CPNE5, CD3E, PCYOX1L, RRAD, COL16A1, BEX2, COL3A1, MXRA8, CD200, ZG16B, GPC3, IGHG2, COL4A2, IGHG4, SSC5D, GAL3ST4, LGALS3, GZMK, BACE2, DPPA4, FBLIM1, OLR1, PDE7A, GEM, TMEM132A, CREB3L1, APOBEC3B, MT1B, CD8B, SLITRK3, RGS4, MAT1A, COL5A1, SEPTIN8, CXCR4, CHI3L1, and TFF2. All of these genes were upregulated with fibrosis histopathology except for EGFLAM, CLEC4M, DPPA4, MT1B, SLITRK3, MAT1A, and TFF2 that were downregulated.

The following fibrosis panel genes were found to be common to the results obtained with just the GHS and REGN data with that for all data: ADRA2A, AEBP1, AKR1B10, ANO9, ANTXR1, AP1M2, AQP1, BACE2, BEX2, BHLHE22, BICC1, C7, CCDCl46, CCDC80, CCL19, CCL21, CCR2, CCR6, CD1E, CD24, CD40LG, CD5, CDH6, CFAP221, CFTR, CHI3L1, CKMT2, CLDN10, CLDN11, CLIC6, COL10A1, COL16A1, COL1A1, COL3A1, COL5A1, COMP, CPZ, CTHRC1, CTSK, CXCL6, CXCR3, DCDC2, DKK3, DPPA4, DPT, DPYSL3, DTNA, EEF1A2, EFEMPI, EGFLAM, EPCAM, EPHA3, F13A1, F3, FAP, FBLN2, FBLN5, GEM, GJA5, GPC3, GPC4, GPRC5B, GSN, HKDC1, IGHG1, IGLC3, ITGBL1, LAMA2, LAMC3, LAYN, LEF1, LOXL1, LOXL4, LRRC1, LTBP2, LUM, LXN, MAT1A, MDFI, MFAP2, MFAP4, MGP, MMP2, MMP7, MOXD1, MUC6, NALCN, NELL2, NPNT, OLR1, OMG, PAPLN, PAQR5, PCYOX1L, PDGFD, PDZK1IP1, PHLDA3, PLCXD3, PLPP4, PODN, PTGDS, PTK7, RAB25, RASL11B, RGS4, RRAD, SCTR, SEPTIN8, SEZ6L2, SIRPG, SIT1, SLAMF1, SLC7A6, SMOC2, SNAP25, SOD3, SOX9, SPINTi, SPON1, SPP1, STMN2, STMN3, SUSD2, THBS2, THY1, TMPRSS3, TRAC, TRAT1, TYMS, UBASH3A, UBD, VEPH1, VTCN1, VWF, WNT4, and ZMAT3. The following fibrosis panel genes were found in results obtained for all data, not the GHS and REGN data: ANKRD29, BOC, CCL20, CH25H, COL1A2, CXCL1, CXCL8, FXYD2, HSPB2, IGFBP7, KRT7, LGR6, NFASC, PDGFA, SEMA3G, TACSTD2, and WFDC2. The following fibrosis panel genes were found in results obtained for the GHS and REGN data, not all data: ADAM28, APOBEC3B, BHLHE41, CD2, CD200, CD27, CD28, CD3D, CD3E, CD8B, CD96, CLEC4M, COL4A2, CPNE5, CREB3L1, CXCR4, DCN, EDN2, FBLIM1, GAL3ST4, GZMK, IGHA1, IGHG2, IGHG3, IGHG4, IGKC, IGLC1, IGLC2, IGLL5, IL7R, KCNN4, LBH, LGALS3, MAP9, MS4A1, MT1B, MXRA8, MZB1, NTS, PCNX2, PDE7A, POU2AF1, RNASE1, SAMD11, SCRN1, SLC1A7, SLITRK3, SSC5D, TC2N, TESPA1, TFF2, TMEM132A, TMEM159, TNFRSF13B, TNFRSF17, TRBC1, TRBC2, TTC9, and ZG16B.

The following fibrosis gene panel was selected using only the GHS and Govaere data, in ranked order: STMN2, FAP, MOXD1, ITGBL1, COL10A1, CLIC6, EFEMPI, THY1, MMP7, MDFI, ADRA2A, LOXL1, LTBP2, SCTR, LOXL4, NALCN, LAMC3, VTCN1, CLDN11, LUM, CFAP221, THBS2, UBD, PAPLN, PDZK1IP1, CTHRC1, SMOC2, MFAP2, PTK7, CDH6, AEBP1, SPON1, PTGDS, DPT, MMP2, DCDC2, RASL11B, EPCAM, CPZ, SPINTi, SOX9, CD24, BICC1, CFTR, FBLN5, WNT4, LRRC1, BHLHE22, PODN, DKK3, TMPRSS3, F3, RAB25, COL16A1, LAMA2, GPC4, MFAP4, EPHA3, DPPA4, BOC, SNAP25, CXCL6, AKR1B10, DTNA, SOD3, GJA5, COMP, PLPP4, CCL20, SEZ6L2, GSN, CCDC80, PAQR5, NFASC, AP1M2, OLR1, PLCXD3, SUSD2, AQP1, RGS4, LEF1, CTSK, GPRC5B, PDGFA, COL1A2, DPYSL3, GEM, SEMA3G, COL5A1, COL3A1, MGP, COL1A1, LAYN, CLDN10, CKMT2, NPNT, SPP1, CD1E, LGR6, CCL21, HKDC1, KRT7, CCL19, CREB3L1, C7, LXN, SLC7A6, GPC3, EGFLAM, NELL2, CYP2C19, FBLIM1, IGFBP7, JAG2, VEPH1, RERGL, NAV3, VCAN, WFDC2, SAMD11, ITGA3, GAL3ST4, ANO9, MUC6, B3GNT3, CDH11, VWF, NCAM2, TRO, F13A1, TACSTD2, ANKRD29, CXCL1, MST1R, SLC2A14, MAP1B, PHLDA3, C1orf198, ANTXR1, CCDCl46, RRAD, MXRA8, PMEPA1, JAG1, NRIP2, TMEM132A, STMN3, GABRE, LGALS3, ID4, FBLN2, BACE2, BEX2, ETV4, CXCR3, INAVA, SOX4, PLPP2, FXYD2, EEF1A2, PCYOX1L, WNK2, CHI3L1, HEPH, VSIG2, CERCAM, PRICKLE1, COL4A2, TYMS, TPM2, SSC5D, CD40LG, CH25H, TRNP1, TRAC, CD5, CCN5, SPHK1, SIT1, UBASH3A, FCGR1A, HAAO, COL28A1, MAT1A, AL583836.1, RGCC, CXCL8, SEPTIN8, ZG16B, SLCO1A2, and ALDH2. All of these genes were upregulated with fibrosis histopathology except for DPPA4, EGFLAM, CYP2C19, NCAM2, HAAO, COL28A1, MAT1A, AL583836.1, SLCO1A2, and ALDH2 that were downregulated.

The following fibrosis panel genes were found to be common to the results obtained with just the GHS and Govaere data with that for all data: ADRA2A, AEBP1, AKR1B10, ANKRD29, ANO9, ANTXR1, AP1M2, AQP1, BACE2, BEX2, BHLHE22, BICC1, BOC, C7, CCDCl46, CCDC80, CCL19, CCL20, CCL21, CD1E, CD24, CD40LG, CD5, CDH6, CFAP221, CFTR, CH25H, CHI3L1, CKMT2, CLDN10, CLDN11, CLIC6, COL10A1, COL16A1, COL1A1, COL1A2, COL3A1, COL5A1, COMP, CPZ, CTHRC1, CTSK, CXCL1, CXCL6, CXCL8, CXCR3, DCDC2, DKK3, DPPA4, DPT, DPYSL3, DTNA, EEF1A2, EFEMPI, EGFLAM, EPCAM, EPHA3, F13A1, F3, FAP, FBLN2, FBLN5, FXYD2, GEM, GJA5, GPC3, GPC4, GPRC5B, GSN, HKDC1, IGFBP7, ITGBL1, KRT7, LAMA2, LAMC3, LAYN, LEF1, LGR6, LOXL1, LOXL4, LRRC1, LTBP2, LUM, LXN, MAT1A, MDFI, MFAP2, MFAP4, MGP, MMP2, MMP7, MOXD1, MUC6, NALCN, NELL2, NFASC, NPNT, OLR1, PAPLN, PAQR5, PCYOX1L, PDGFA, PDZK11P1, PHLDA3, PLCXD3, PLPP4, PODN, PTGDS, PTK7, RAB25, RASL11B, RGS4, RRAD, SCTR, SEMA3G, SEPTIN8, SEZ6L2, SIT1, SLC7A6, SMOC2, SNAP25, SOD3, SOX9, SPINTi, SPON1, SPP1, STMN2, STMN3, SUSD2, TACSTD2, THBS2, THY1, TMPRSS3, TRAC, TYMS, UBASH3A, UBD, VEPH1, VTCN1, VWF, WFDC2, and WNT4. The following fibrosis panel genes were found in results obtained for all data, not the GHS and Govaere data: CCR2, CCR6, HSPB2, IGHG1, IGLC3, OMG, PDGFD, SIRPG, SLAMF1, TRAT1, and ZMAT3. The following fibrosis panel genes were found in results obtained for the GHS and Govaere data, not all data: AL583836.1, ALDH2, B3GNT3, C1orf198, CCN5, CDH11, CERCAM, COL28A1, COL4A2, CREB3L1, CYP2C19, ETV4, FBLIM1, FCGR1A, GABRE, GAL3ST4, HAAO, HEPH, ID4, INAVA, ITGA3, JAG1, JAG2, LGALS3, MAP1B, MST1R, MXRA8, NAV3, NCAM2, NRIP2, PLPP2, PMEPA1, PRICKLE1, RERGL, RGCC, SAMD11, SLC2A14, SLCO1A2, SOX4, SPHK1, SSC5D, TMEM132A, TPM2, TRNP1, TRO, VCAN, VSIG2, WNK2, and ZG16B.

The following fibrosis gene panel was selected using only the REGN and Govaere data, in ranked order: STMN2, FAP, SPATA21, ITGBL1, MOXD1, SCTR, COL10A1, NALCN, CLIC6, MMP7, EFEMPI, LTBP2, EPCAM, THY1, LUM, DTNA, MDFI, DCDC2, LOXL1, CDH6, LRRC1, VTCN1, CLDN11, PLCXD3, CD24, PTGDS, DPT, BHLHE22, RASL11B, LAMC3, CFTR, RAB25, AEBP1, FBLN5, BICC1, PAPLN, ADRA2A, WNT4, SOX9, TMPRSS3, EPHA3, SPINTi, PODN, DKK3, CPZ, ESRP1, C7, MFAP4, GPC4, BEX2, GRHL2, CXCL6, AKR1B10, CFAP221, UBD, CXCL1, LOXL4, F3, SPON1, SMOC2, CTHRC1, GJA5, CXCL8, KRT7, LAMA2, PDZK11P1, THBS2, CH25H, RGS4, SOD3, TACSTD2, CKMT2, VEPH1, ACKR1, PAQR5, COL15A1, SUSD2, ANKRD29, AMPD1, AQP1, GEM, GSN, TMEM125, LXN, MFAP2, PLPP4, CLDN10, FXYD2, MMP2, CD1E, OLR1, WFDC2, MGP, ANO9, SNAP25, LEF1, CABYR, CCL19, HKDC1, GPRC5B, GPC3, SLC7A6, CCDC80, CCL21, IGLC2, EGFLAM, IGLC3, MZB1, NAV3, IGLC1, AP1M2, ELOVL7, NELL2, CACNAIC, IGKC, PDGFA, FAM3B, IGHG1, PBX4, SLC35F2, PTK7, RCAN3, COMP, MYEF2, TNFRSF17, WNT10A, MAP1B, VWF, SOX4, ANTXR1, ETV4, SEZ6L2, TRNP1, CCDCl46, NFASC, PHLDA3, PIWIL4, TC2N, SIRPG, CCR2, COL1A2, PRSS22, VSIG2, RRAD, SEMA3G, MAP9, PLPP2, FCRL5, IGLL5, BACE2, COL16A1, GABRE, CHI3L1, SPP1, PMEPA1, GLS, PCYOX1L, CPNE5, IGHG2, NCAM2, SH3YL1, ZNF607, B3GNT3, SLAMF1, ADCY1, TRAC, POU2AF1, JAG1, DPPA4, TNFRSF13B, TRBC2, CD27, GABRB3, EEF1A2, RGCC, AKAP7, BCL11B, CXCR4, BOC, CD40LG, TYMS, CD3D, CCL20, INAVA, EDN2, SPHK1, OMG, MUC6, GPR174, TPM1, CD2, CLCF1, MAT1A, ZMAT3, TRBC1, SIT1, SLC2A14, TRAT1, SEPTIN8, GZMK, CD3E, and IL7R. All of these genes were upregulated with fibrosis histopathology except for EGFLAM, NCAM2, ADCY1, DPPA4, and MAT1A that were downregulated.

The following fibrosis panel genes were found to be common to the results obtained with just the REGN and Govaere data with that for all data: ADRA2A, AEBP1, AKR1B10, ANKRD29, ANO9, ANTXR1, AP1M2, AQP1, BACE2, BEX2, BHLHE22, BICC1, BOC, C7, CCDCl46, CCDC80, CCL19, CCL20, CCL21, CCR2, CD1E, CD24, CD40LG, CDH6, CFAP221, CFTR, CH25H, CHI3L1, CKMT2, CLDN10, CLDN11, CLIC6, COL10A1, COL16A1, COL1A2, COMP, CPZ, CTHRC1, CXCL1, CXCL6, CXCL8, DCDC2, DKK3, DPPA4, DPT, DTNA, EEF1A2, EFEMPI, EGFLAM, EPCAM, EPHA3, F3, FAP, FBLN5, FXYD2, GEM, GJA5, GPC3, GPC4, GPRC5B, GSN, HKDC1, IGHG1, IGLC3, ITGBL1, KRT7, LAMA2, LAMC3, LEF1, LOXL1, LOXL4, LRRC1, LTBP2, LUM, LXN, MAT1A, MDFI, MFAP2, MFAP4, MGP, MMP2, MMP7, MOXD1, MUC6, NALCN, NELL2, NFASC, OLR1, OMG, PAPLN, PAQR5, PCYOX1L, PDGFA, PDZK1IP1, PHLDA3, PLCXD3, PLPP4, PODN, PTGDS, PTK7, RAB25, RASL11B, RGS4, RRAD, SCTR, SEMA3G, SEPTIN8, SEZ6L2, SIRPG, SIT1, SLAMF1, SLC7A6, SMOC2, SNAP25, SOD3, SOX9, SPINTi, SPON1, SPP1, STMN2, SUSD2, TACSTD2, THBS2, THY1, TMPRSS3, TRAC, TRAT1, TYMS, UBD, VEPH1, VTCN1, VWF, WFDC2, WNT4, and ZMAT3. The following fibrosis panel genes were found in results obtained for all data, not the REGN and Govaere data: CCR6, CD5, COL1A1, COL3A1, COL5A1, CTSK, CXCR3, DPYSL3, F13A1, FBLN2, HSPB2, IGFBP7, LAYN, LGR6, NPNT, PDGFD, STMN3, and UBASH3A. The following fibrosis panel genes were found in results obtained for the REGN and Govaere data, not all data: ACKR1, ADCY1, AKAP7, AMPD1, B3GNT3, BCL11B, CABYR, CACNAIC, CD2, CD27, CD3D, CD3E, CLCF1, COL15A1, CPNE5, CXCR4, EDN2, ELOVL7, ESRP1, ETV4, FAM3B, FCRL5, GABRB3, GABRE, GLS, GPR174, GRHL2, GZMK, IGHG2, IGKC, IGLC1, IGLC2, IGLL5, IL7R, INAVA, JAG1, MAP1B, MAP9, MYEF2, MZB1, NAV3, NCAM2, PBX4, PIWIL4, PLPP2, PMEPA1, POU2AF1, PRSS22, RCAN3, RGCC, SH3YL1, SLC2A14, SLC35F2, SOX4, SPATA21, SPHK1, TC2N, TMEM125, TNFRSF13B, TNFRSF17, TPM1, TRBC1, TRBC2, TRNP1, VSIG2, WNT10A, and ZNF607.

FIGS. 4 and 5 demonstrate that if 30% of the data was separated out into a testing dataset, and then, the same methodology was used on the 70% training dataset, similar gene panels are obtained that perform similarly well on the testing dataset.

The following fibrosis gene panel was selected using the 70% training data, in ranked order: STMN2, FAP, MOXD1, ITGBL1, SCTR, COL10A1, NALCN, EFEMPI, MMP7, LOXL1, MDFI, LUM, CLIC6, LTBP2, THY1, LAMC3, CLDN11, BHLHE22, DTNA, VTCN1, DCDC2, ADRA2A, AEBP1, PAPLN, SOX9, CDH6, CTHRC1, CFAP221, EPCAM, CD24, PLCXD3, CPZ, EPHA3, LRRC1, THBS2, BICC1, LAMA2, FBLN5, LEF1, PODN, RASL11B, DKK3, MFAP4, C7, GPC4, CFTR, CXCL6, GJA5, PTGDS, LOXL4, WNT4, DPT, SMOC2, MFAP2, SUSD2, SPON1, F3, SPINTi, TMPRSS3, MMP2, SOD3, RAB25, LXN, GSN, LAYN, AQP1, ANTXR1, AKR1B10, PHLDA3, UBD, CCL19, RGS4, CXCL1, PDZK1IP1, CCL21, LIF, COL16A1, GPRC5B, GEM, PTK7, CD40LG, SNAP25, BEX2, CKMT2, IGHA1, CCDC80, DPYSL3, PLPP4, PAQR5, COL1A2, ANO9, VEPH1, GLIS2, CLDN10, NFASC, F13A1, AP1M2, RRAD, HSPB8, SLAMF1, IGKC, COL4A3, COL5A1, OLR1, TACSTD2, HKDC1, IGLC3, NTS, CXCL8, BOC, TRAC, C1orf198, NPNT, PDGFA, COL1A1, FBLN2, COMP, CD5, PDGFD, KRT7, IGHG1, SPP1, SLC7A6, NELL2, ID4, CCL20, EGFLAM, WFDC2, DPPA4, EEF1A2, CCR2, BHLHE41, MEI1, COL4A4, VWF, SEMA3G, CHI3L1, SLC34A2, STMN3, SSPN, OMG, TYMS, ITGA3, ADAM28, IGLC7, MZB1, SAMD11, ABCC4, TRAT1, LGR6, MYEF2, DCN, LBH, SIT1, RERGL, CD27, FAM3B, MGP, PRSS22, ZMAT3, CXCR3, CH25H, TNFRSF17, IGLL5, CD1E, GPC3, CLDN4, FMO2, IGHG3, PCYOX1L, TPM1, and SLC1A7. All of these genes were upregulated with fibrosis histopathology except for EGFLAM and DPPA4 that were downregulated.

The following fibrosis panel genes were found to be common to the results obtained for the 70% training data with that for all data: ADRA2A, AEBP1, AKR1B10, ANO9, ANTXR1, AP1M2, AQP1, BEX2, BHLHE22, BICC1, BOC, C7, CCDC80, CCL19, CCL20, CCL21, CCR2, CD1E, CD24, CD40LG, CD5, CDH6, CFAP221, CFTR, CH25H, CHI3L1, CKMT2, CLDN10, CLDN11, CLIC6, COL10A1, COL16A1, COL1A1, COL1A2, COL5A1, COMP, CPZ, CTHRC1, CXCL1, CXCL6, CXCL8, CXCR3, DCDC2, DKK3, DPPA4, DPT, DPYSL3, DTNA, EEF1A2, EFEMPI, EGFLAM, EPCAM, EPHA3, F13A1, F3, FAP, FBLN2, FBLN5, GEM, GJA5, GPC3, GPC4, GPRC5B, GSN, HKDC1, IGHG1, IGLC3, ITGBL1, KRT7, LAMA2, LAMC3, LAYN, LEF1, LGR6, LOXL1, LOXL4, LRRC1, LTBP2, LUM, LXN, MDFI, MFAP2, MFAP4, MGP, MMP2, MMP7, MOXD1, NALCN, NELL2, NFASC, NPNT, OLR1, OMG, PAPLN, PAQR5, PCYOX1L, PDGFA, PDGFD, PDZK11P1, PHLDA3, PLCXD3, PLPP4, PODN, PTGDS, PTK7, RAB25, RASL11B, RGS4, RRAD, SCTR, SEMA3G, SIT1, SLAMF1, SLC7A6, SMOC2, SNAP25, SOD3, SOX9, SPINTi, SPON1, SPP1, STMN2, STMN3, SUSD2, TACSTD2, THBS2, THY1, TMPRSS3, TRAC, TRAT1, TYMS, UBD, VEPH1, VTCN1, VWF, WFDC2, WNT4, and ZMAT3. The following fibrosis panel genes were found in results obtained for all data, not the 70% training data: ANKRD29, BACE2, CCDCl46, CCR6, COL3A1, CTSK, FXYD2, HSPB2, IGFBP7, MAT1A, MUC6, SEPTIN8, SEZ6L2, SIRPG, and UBASH3A. The following fibrosis panel genes were found in results obtained for the 70% training data, not all data: ABCC4, ADAM28, BHLHE41, C1orf198, CD27, CLDN4, COL4A3, COL4A4, DCN, FAM3B, FMO2, GLIS2, HSPB8, ID4, IGHA1, IGHG3, IGKC, IGLC7, IGLL5, ITGA3, LBH, LIF, MEI1, MYEF2, MZB1, NTS, PRSS22, RERGL, SAMD11, SLC1A7, SLC34A2, SSPN, TNFRSF17, and TPM1.

The following inflammation gene panel was selected using the 70% training data, in ranked order: LPL, TREM2, EEF1A2, FABP4, STMN2, SPP1, CAPG, LOXL4, EMILIN2, FABP5, SLAMF8, CD300LB, CLDN11, MMP9, COL1A1, OLR1, C15orf48, HS3ST2, UBD, LGALS3, THY1, DTNA, DHRS9, CPZ, CXCL10, BCAT1, COL3A1, SPATA21, COL1A2, ITGBL1, KCNN4, RTN1, RASL10B, MOXD1, LYZ, DUSP8, PDGFA, CCL20, IL32, CENPV, CD24, LOXL1, EFEMPI, THBS2, AEBP1, CD52, TTC9, ITGAX, LAIR1, LSP1, ALOX5AP, GPNMB, LTBP2, TP5313, LRRC1, DNAJC5B, PTK7, PODN, CD37, VCAN, KRT23, MTHFD2, PRAMEF10, FSTL3, RRAD, MICAL1, MDFI, CCL17, BHLHE22, SNAP25, MRAS, PTGDS, KLHL29, F13A1, DNAJC12, TRIM31, SLC2A14, CXCR4, PLXNC1, SLC1A7, PAPLN, LAMA2, LAMC3, CMYA5, B3GNT5, GAPT, ACADSB, TNFRSF4, JAML, IL411, PFKP, DGKA, PBX4, TNFRSF18, GPR132, JAK3, CD40LG, RPS6KA1, KRTCAP3, GPR183, GM2A, LAMA3, CD1C, COL8A2, CCR5, ABR, FCAMR, TRAF1, CYP2C19, LGALS2, SLC16A3, IL27RA, FBLN2, MMP7, FGR, PDCD1, ABCA7, FCN1, LRFN4, CACNB1, KBTBD11, HAPLN3, CHIT1, GJA5, ANO9, GRAMD1A, TRPM2, NCF2, MMP14, ATP2A3, NKD2, PLD4, BAG3, SUSD2, STMN3, LILRB3, MACO1, EPHA3, SAMD11, HRH2, SIRPG, PRAMEF33, UNC119, PDGFRB, KAZALD1, AKAP9, LBH, BATF, PI3, and MT1B. All of these genes were upregulated with inflammation histopathology except for RASL10B, CENPV, DNAJC12, CMYA5, ACADSB, KRTCAP3, CYP2C19, KBTBD11, MACO1, AKAP9, and MT1B that were downregulated.

The following inflammation panel genes were found to be common to the results obtained for the 70% training data with that for all data: ABR, ACADSB, AEBP1, ALOX5AP, ANO9, B3GNT5, BATF, BCAT1, BHLHE22, C15orf48, CACNB1, CAPG, CCL17, CCL20, CCR5, CD24, CD300LB, CD37, CD40LG, CD52, CENPV, CHIT1, CLDN11, CMYA5, COL1A1, COL1A2, COL3A1, CPZ, CXCL10, CYP2C19, DGKA, DHRS9, DNAJC12, DNAJC5B, DTNA, DUSP8, EFEMPI, EMILIN2, EPHA3, FABP4, FABP5, FBLN2, FCAMR, FGR, FSTL3, GAPT, GJA5, GPNMB, HAPLN3, HS3ST2, IL32, IL411, ITGAX, ITGBL1, JAK3, KCNN4, KLHL29, KRT23, KRTCAP3, LAIR1, LAMA2, LAMA3, LAMC3, LGALS2, LGALS3, LILRB3, LOXL1, LOXL4, LPL, LRFN4, LRRC1, LSP1, LTBP2, LYZ, MACO1, MDFI, MICAL1, MMP14, MMP7, MMP9, MOXD1, MRAS, MT1B, MTHFD2, NCF2, OLR1, PAPLN, PBX4, PDCD1, PDGFA, PLD4, PLXNC1, PODN, PRAMEF10, PRAMEF33, PTGDS, RASL10B, RPS6KA1, RRAD, RTN1, SAMD11, SIRPG, SLAMF8, SLC16A3, SLC1A7, SLC2A14, SNAP25, SPATA21, SPP1, STMN2, SUSD2, THBS2, THY1, TNFRSF4, TP5313, TRAF1, TREM2, TRIM31, TRPM2, TTC9, UBD, and VCAN. The following inflammation panel genes were found in results obtained for all data, not the 70% training data: ADAM28, AQP8, CARMIL2, CCL22, CCR2, CCR7, CD1E, CD2, CD28, CD48, CD5, CD6, CD96, CDH6, CLIC6, COMP, CXCR3, EGFLAM, GABRE, GEM, GPR174, GRAP2, LCK, LUM, MS4A14, NELL2, PTPN7, RAB7B, RGS10, SCTR, SIT1, SLC7A6, SLCO1A2, TMEM164, VIL1, WFDC2, and WNT4. The following inflammation panel genes were found in results obtained for the 70% training data, not all data: ABCA7, AKAP9, ATP2A3, BAG3, CD1C, COL8A2, CXCR4, EEF1A2, F13A1, FCN1, GM2A, GPR132, GPR183, GRAMD1A, HRH2, IL27RA, JAML, KAZALD1, KBTBD11, LBH, NKD2, PDGFRB, PFKP, PI3, PTK7, STMN3, TNFRSF18, and UNC119.

### Example 3: Evaluation of Fibrosis Gene Panel in External Cohort

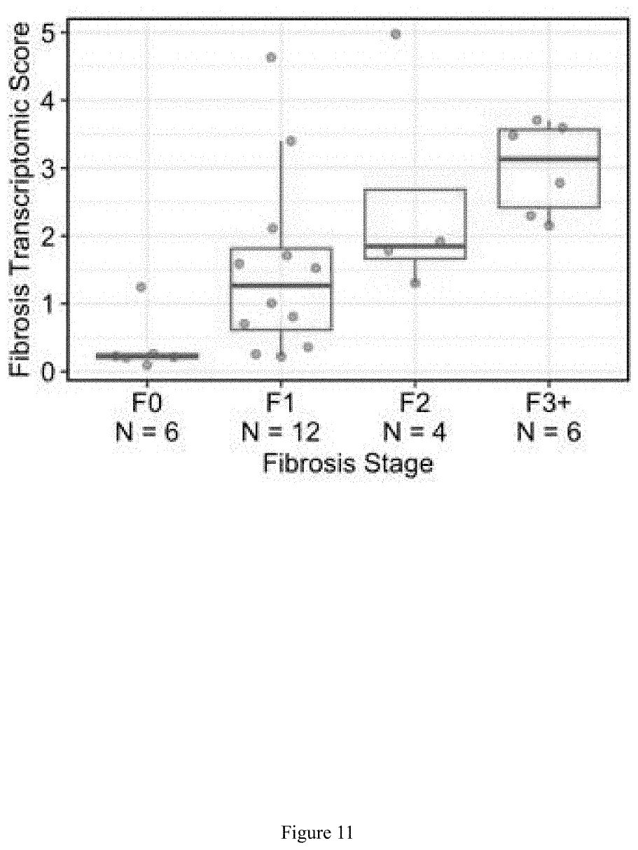

The fibrosis gene panel was evaluated in a liver biopsy RNA-seq dataset from 28 participants. These participants had the following fibrosis histopathology scores: 6 were F0, 12 were F1, 4 were F2, and 5 were F3, and 1 was F4. One liver biopsy was RNA sequenced from each participant. Using a similar procedure as described in Example 2, the fibrosis transcriptomic score (TS) was calculated for each sample using single-sample gene set enrichment analysis (GSEA2) with the fibrosis gene panel of 153 genes. FIG. 11 shows the fibrosis transcriptomic score (TS) for each participant along their fibrosis histopathology scores. The performance of the computed fibrosis transcriptomic scores was evaluated using a non-parametric statistical test of Wilcoxon rank-sum test, comparing the fibrosis transcriptomic scores of samples with histopathology F2+ (high fibrosis) vs F0/F1 (no/low fibrosis). The result of the Wilcoxon rank-sum test was highly significant (p=4.7E-4), indicating that the fibrosis gene panel distinguished high vs no/low fibrosis in the RNA-seq of these liver biopsies.

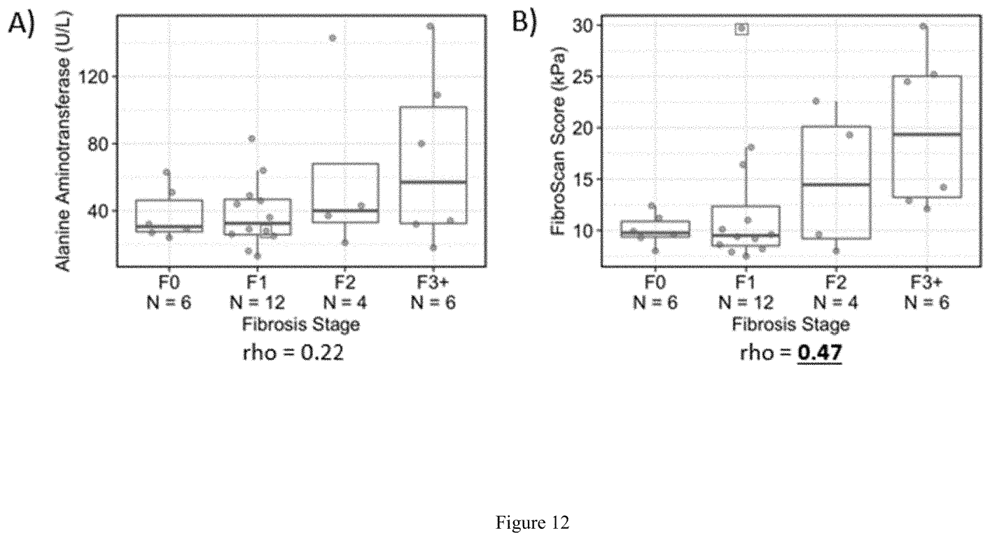

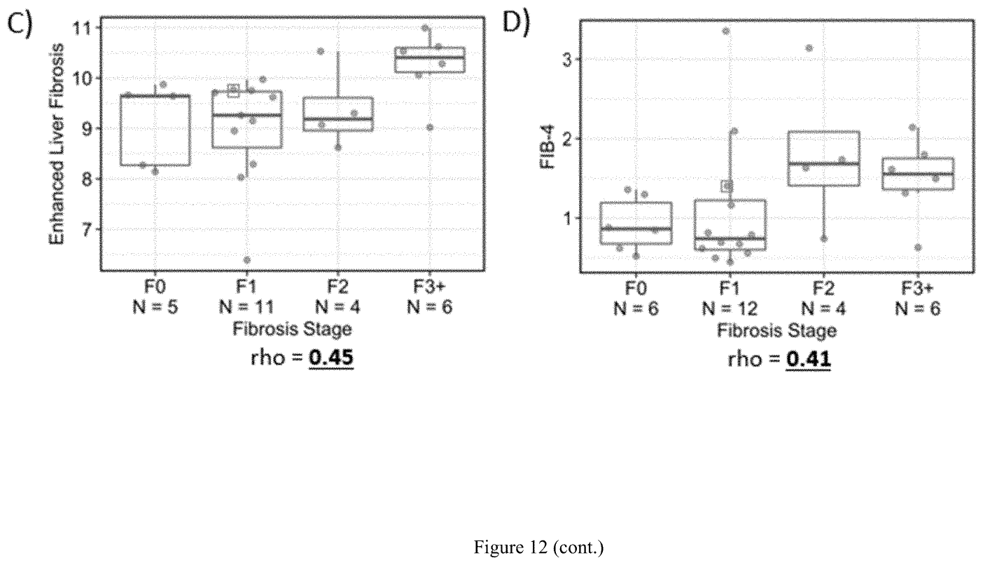

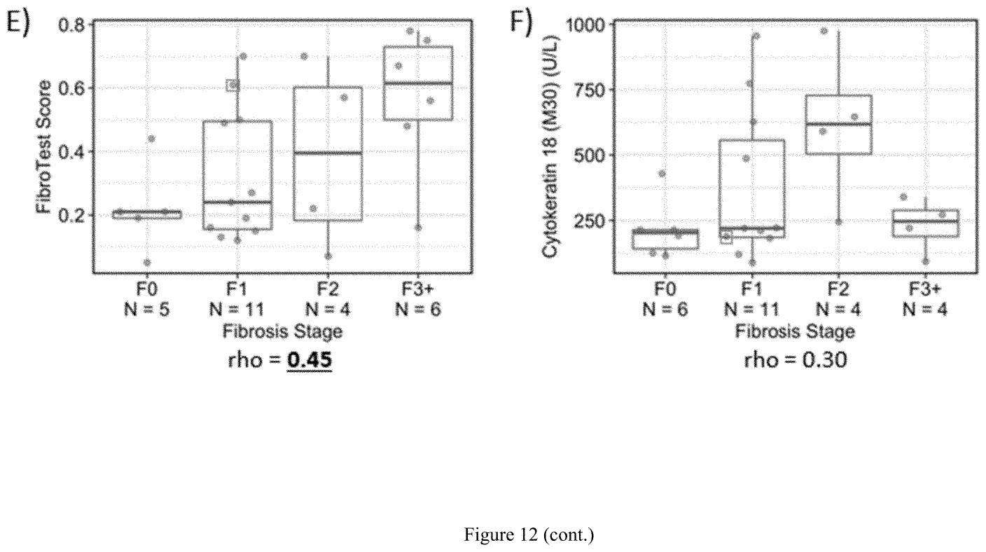

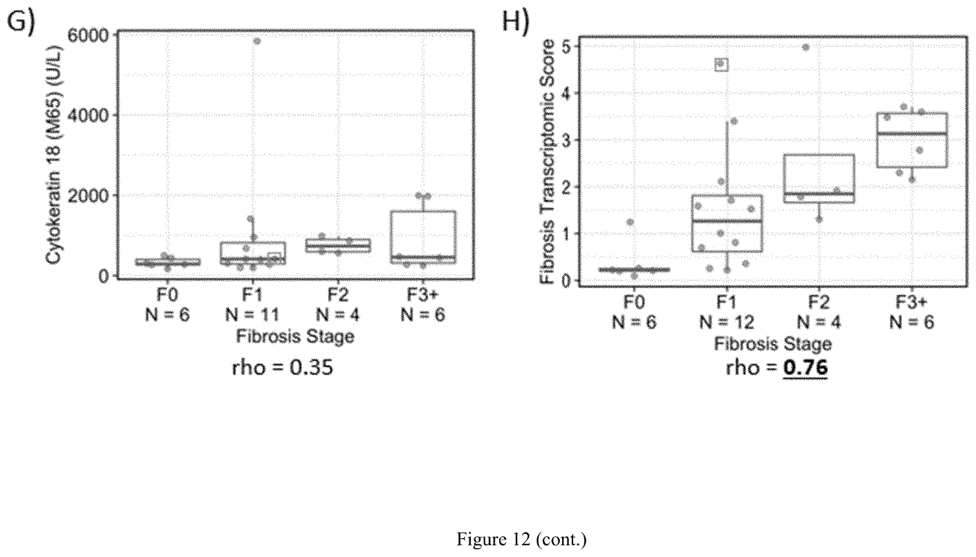

FIG. 12 (Panels A, B, C, D, E, F, G, and H) shows the performance of the fibrosis gene panel was compared against other clinical biomarkers of liver fibrosis and NASH disease including alanine aminotransferase (ALT), non-invasive liver transient elastography FibroScan®, Enhanced Liver Fibrosis (ELF)™, FIB-4, and FibroTest™, caspase-cleaved cytokeratin 18 (M30), and total cytokeratin 18 (M65). FibroTest™ uses an algorithm based on five serum biomarkers (gamma-glutamyltransferase (GGT), total bilirubin, alpha-2-macroglobulin (A2M), apolipoprotein A1, and haptoglobin), weighted based on participant's age and sex. Enhanced Liver Fibrosis (ELF)™ uses an algorithm based on three serum biomarkers: hyaluronic acid (HA), procollagen type III N-terminal peptide (PIIINP), tissue inhibitor of matrix metaloproteinase-1 (TIMP-1). FIB-4 uses an algorithm based on three serum biomarkers (alanine transaminase (AST), alanine aminotransferase (ALT), and platelet count) and the participant's age. The Spearman's rank correlation coefficient (rho) and statistical significance (p value) of each biomarker against fibrosis histopathology was as follows: ALT (rho=0.22, p=0.25), FibroScan® (rho=0.47, p=0.012), ELF™ score (rho=0.45, p=0.020), FIB-4 (rho=0.41, p=0.031), FibroTest™ score (rho=0.45, p=0.022), cytokeratin 18 (M30) (rho=0.30, p=0.14), cytokeratin 18 (M65) (rho=0.35, p=0.071), and fibrosis transcriptomic score (TS) described herein (rho=0.76, p=2.5E-06). Of these biomarkers, the fibrosis transcriptomic score (TS) had the highest Spearman's rho with the fibrosis histopathology. The participant with the highest fibrosis transcriptomic score in the F1 fibrosis histopathology category is indicated with a box around the dot across the panels and had high scores (top quartile) for FibroScan®, ELF™, FIB-4, FibroTest™, suggesting that this participant may have had more advanced fibrosis than indicated by the histopathology reading.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

1. A method of treating a subject having liver inflammation and/or liver fibrosis, the method comprising:
administering a therapeutic agent that treats or inhibits liver inflammation and/or liver fibrosis and/or conducting a surgery on the subject when the subject's Transcriptome Score (TS) is greater than a threshold TS determined from a reference population of subjects without liver inflammation and/or without liver fibrosis;
wherein the TS comprises a value determined from RNA expression in a biological sample from the subject;
wherein the threshold TS is determined by identifying a gene having a median transcript per million (TPM) value of 0.5; and
wherein the therapeutic agent comprises an HSD17B13 inhibitor, a PNPLA3 inhibitor, a CIDEB inhibitor, or any combination thereof.

2. The method according to claim 1, wherein the RNA expression comprises a quantification of RNA expression of at least one gene.

3. The method according to claim 2, wherein the at least one gene comprises a protein-coding gene, a long non-coding RNA, a mitochondrial rRNA, a mitochondrial tRNA, an rRNA, a ribozyme, a B-cell receptor subunit constant gene, and/or a T-cell receptor subunit constant gene.

4. The method according to claim 2, wherein the at least one gene comprises 10 genes.

5. The method according to claim 2, wherein the at least one gene comprises 50 genes.

6. The method according to claim 2, wherein the at least one gene comprises 100 genes.

7. The method according to claim 2, wherein the at least one gene comprises 200 genes.

8. The method according to claim 2, wherein the at least one gene comprises 1,000 genes.

9-15. (canceled)

16. The method according to claim 2, wherein the reference population of subjects without liver inflammation and/or without liver fibrosis comprises a reference population of subjects without liver fibrosis, and wherein the at least one gene is upregulated with respect to that of the reference population of subjects without liver fibrosis.

17. (canceled)

18. The method according to claim 2, wherein the reference population of subjects without liver inflammation and/or without liver fibrosis comprises a reference population of subjects without liver inflammation, and wherein the at least one gene is downregulated with respect to that of the reference population of subjects without liver fibrosis.

19-26. (canceled)

27. The method according to claim 2, wherein the reference population of subjects without liver inflammation and/or without liver fibrosis comprises a reference population of subjects without liver inflammation, and wherein the at least one gene is upregulated with respect to that of in the reference population of subjects without liver inflammation.

28. (canceled)

29. The method according to claim 2, wherein the reference population of subjects without liver inflammation and/or without liver fibrosis comprises a reference population of subjects without liver inflammation, and wherein the at least one gene is downregulated with respect to that of the reference population of subjects without liver inflammation.

30. (canceled)

31. The method according to claim 2, wherein the determination of the suitability for inclusion of the gene in a panel comprises calculating an area under a curve for the at least one gene.

32-40. (canceled)

41. The method according to claim 2, wherein the quantification comprises determining an RNA expression value for the at least one gene with respect to that of the reference population of subjects without liver inflammation and/or without liver fibrosis.

42. The method according to claim 2, wherein the at least one gene comprises a plurality of genes, wherein the quantification comprises determining an expression value for each gene of the plurality of genes with respect to that of the reference population of subjects without liver inflammation and/or without liver fibrosis, thereby generating a plurality of stratified values.

43. The method according to claim 42, wherein the quantification further comprises determining an expression value for the at least one gene with respect to that of the reference population of subjects without liver inflammation and/or without liver fibrosis, and wherein the TS is within a percentile of the plurality of stratified values.

44. The method according to claim 43, wherein the TS is within the fiftieth percentile.

45. The method according to claim 43, wherein the TS is within the sixtieth percentile.

46. The method according to claim 43, wherein the TS is within the seventieth percentile.

47. The method according to claim 43, wherein the TS is within the seventy-fifth percentile.

48. The method according to claim 43, wherein the TS is within the eightieth percentile.

49. The method according to claim 43, wherein the TS is within the ninetieth percentile.

50. The method according to claim 43, wherein the TS is within the ninety-fifth percentile.

51. The method according to claim 1, wherein the threshold TS comprises a normalized enrichment score.

52. The method according to claim 1, wherein the liver inflammation comprises inflammation associated with alcohol abuse, an alpha-1 antitrypsin deficiency, an autoimmune reaction, a decrease of a blood flow to the liver, a drug, a toxin, hemochromatosis, obstructive jaundice, a viral infection, Wilson's disease, or nonalcoholic fatty liver disease.

53. The method according to claim 52, wherein the viral infection comprises a hepatitis A viral infection, a hepatitis B viral infection, a hepatitis C viral infection, a hepatitis D viral infection, or a hepatitis E viral infection.

54. The method according to claim 52, wherein the nonalcoholic fatty liver disease comprises liver fibrosis.

55. The method according to claim 1, wherein the liver fibrosis comprises fibrosis associated with alcohol abuse, fibrosis associated with a hepatitis C infection, fibrosis associated with nonalcoholic fatty liver disease, or cirrhosis.

56. The method according to claim 52, wherein the nonalcoholic fatty liver disease comprises nonalcoholic steatohepatitis.

57. (canceled)

58. The method according to claim 1,
wherein:
when the subject's TS is greater at a later time point than an earlier time point, the liver inflammation and/or the liver fibrosis has progressed in the subject; or
when the subject's TS is greater at the earlier time point than the later time point, the liver inflammation and/or the liver fibrosis has regressed in the subject.

59-105. (canceled)
